(19) United States
(12) Patent Application Publication
(10) Pub. No.: US 2003/0027152 A1
(43) Pub. Date: Feb. 6, 2003

(54) Generation of diagnostic tools to assay the human LHX3/P-LIM/LIM-3 factor

(75) Inventors: Simon J. Rhodes, Indianapolis, IN (US); Jeanne L. Bridwell, Indianapolis, IN (US); Bradley C. Meier, Mishawaka, IN (US); Gretchen E. Parker, Indianapolis, IN (US); Jeffrey R. Price, Knightdale, NC (US); Aaron D. Showalter, Indianapolis, IN (US); Kyle W. Sloop, Indianapolis, IN (US)

Correspondence Address: MORGAN, LEWIS & BOCKIUS LLP, 1701 MARKET STREET, PHILADELPHIA, PA, 19103-2921, US

(73) Assignee: Indiana University Advanced Research and Technology Institute

(21) Appl. No.: 09/932,367

(22) Filed: Aug. 17, 2001

Related U.S. Application Data
(63) Continuation of application No. PCT/US00/04424, filed on Feb. 22, 2000.
(60) Provisional application No. 60/121,110, filed on Feb. 22, 1999.

(51) Int. Cl. C12Q 1/68; C07H 21/04; C12P 21/04; C12N 9/00; C12P 21/02; C12N 5/06; C07K 14/435

(52) U.S. Cl. 435/6; 536/23.2; 435/69.7; 435/325; 435/320.1; 435/183; 530/350

(57) ABSTRACT

The present invention relates to mammalian Lhx3 nucleic acids and proteins encoded thereby. More specifically, the invention discloses novel porcine and human Lhx3 genes and isoforms thereof. The invention further relates to genomic mapping of the human Lhx3 coding region to the subtelomeric region of Chromosome 9. The invention also relates to methods and kits relating to the nucleic and amino acids encoding Lhx3 and uses therefor.

### BACKGROUND OF THE INVENTION

The invention involves the identification and characterization of the sequence of the human Lhx3/P-LIM/LIM-3 gene and homologous genes in other mammals. The identification of this gene facilitates the development of diagnostic procedures designed to detect mutations in the gene, which mutations may be responsible for congenital pituitary disease (usually involving short stature and other symptoms) in humans. Further, the invention is useful for the detection of abnormal expression levels of this gene which abnormal levels may be associated with pituitary diseases, disorders, or conditions including tumors.

The molecular mechanisms that regulate the development of complex endocrine organ systems are poorly understood. The pituitary gland represents an important model system to study how regulatory genes direct the determination a differentiation of the individual cell lineages that ultimately comprise a mature organ.

The pituitary gland is a central endocrine organ in vertebrates. Peptide and protein hormones released from the anterior, intermediate and posterior lobes of the gland regulate physiological processes including growth, lactation, homeostasis, the stress response, reproductive development and fitness, and water metabolism. Hormones of the anterior and intermediate lobes are synthesized by specialized cell types that are characterized by the specific hormones that they secrete.

During development, the anterior and intermediate lobes of the pituitary are formed from a fold of oral ectoderm known as Rathke's Pouch (reviewed by Rhodes et al., 1994; Curr. Op. in Genet. and Dev. 4:709-717; Treier and Rosenfeld, 1996, Curr. Opin. Cell Biol. 8:833-843). The mature gland consists of the anterior and intermediate lobes associated with an extension of neuroectoderm from the brain, the posterior pituitary. Six distinct cell types emerge from Rathke's pouch during organogenesis to populate the mature anterior and intermediate lobes of the pituitary (Watkins-Chow et al., 1998, Trends Genet. 14:284-290; Kioussi et al., 1999, Mech. Dev. 81:23-35; Sheng and Westphal, 1999, Trends Genet. 15:236-240). These cells release characteristic protein hormones that regulate growth, lactation, metabolism, reproduction, and the response to stress.

The cell commitment and differentiation decisions that govern the development of the hormone-secreting cells of the anterior and intermediate lobes appear to be regulated by the coordinated actions of pituitary-restricted transcription factors (reviewed by Treier and Rosenfeld, 1996; Curr. Opin. Cell Biol. 8:833-843; Watkins-Chow and Camper, 1998, Trends in Genetics 14:284-290). Protein signaling molecules released from brain neuroectoderm, and from within the developing pituitary itself, may initiate and modulate the actions of these critical transcription factors during embryogenesis (Ericson et al., 1998; Development 125:1005-1015; Treier et al., 1998, Genes Dev. 12:1691-1704).

Proper formation of Rathke's pouch and the subsequent differentiation of pituitary cell types, require the secretion of inductive signaling molecules such as bone morphogenetic protein 2 (BMP-2), BMP-4, and fibroblast growth factor 8 (FGF-8). These molecules are secreted from the ventral diencephalon and developing pituitary to initiate cellular differentiation cascades within embryonic pituitary cells in a spatial and temporal fashion (Ericson et al., 1998, Development 125:1005-1015; Treier et al., 1998, Genes Dev. 12:1691-17046; Takuma et al., 1998, Development 125:4835-4840). The protein signals induce subsequent pituitary cellular specification by activating multiple transcription factors.

Based on their expression patterns, gene knockout experiments, and the analysis of naturally occurring mutants, several transcription factors have been implicated in pituitary organogenesis. They include murine Lhx3 (also known as P-Lim or LIM3), Lhx4/Gsh-4, Isl-1, Krox-24, T/ebp/TTF-1, Hesxl/Rpx, Pitx-1/P-Otx, Pitx2, Prop-1, Pit-1/GFH-1, Brn-4, Six-3, SF-1, Pax-6, Msx-1, .Nkx 3.1, GATA-2, P-Frk, thyrotrope embryonic factor (TEF), and Zn-16 (Watkins-Chow et al., 1998, Trends Genet. 14:284-290; Kioussi et al., 1999, Mech. Dev. 81:23-35; Sheng and Westphal, 1999, Trends Genet. 15:236-240; Ericson et al., 1998, Development 125:1005-1015; Treier et al., 1998, Genes Dev. 12:1691-1704).

Mutations in pituitary transcription factors have been shown to cause pituitary disease. Humans with mutations in the Pit-1 or Prop-1 genes exhibit combined pituitary hormone deficiency (CPHD). Pit-1 is a well characterized member of the family of DNA binding proteins that contain a POU domain, i.e., an acronym consisting of the first letter of the first three family members identified, Pit-1, Oct-1, and Unc-86, as described by Schonemann et al. (1998, Adv. Exp. Med. Biol. 449:39-53). Pit-1 is a pituitary transcription factor that positively autoregulates its own gene and directly activates pituitary trophic hormone genes, including the growth hormone (GH), prolactin (PRL), and thyroid-stimulating hormone beta (TSHβ) genes (as reviewed by Watkins-Chow et al., 1998, Trends Genet. 14:284-290; Kioussi et al., 1999, Mech. Dev. 81:23-35; Sheng and Westphal, 1999, Trends Genet. 15:236-2401; Rhodes and Rosenfeld, 1996, J. Anim. Sci. 74:94-106). Patients with mutated Pit-1 genes have deficiencies in GH, PRL, and TSH. Many types of Pit-1 gene mutations have been identified, including those with autosomal dominant and recessive patterns of inheritance (Rhodes and Rosenfeld, 1996, J. Anim. Sci. 74:94-106; Procter et al., 1998, Hum. Genet. 103:255-272). The Prop-1 (or Prophet of Pit-1) transcription factor is upstream of Pit-1 in the pituitary development cascade (Somson et al., 1996, Nature 384:327-333). In addition to deficiencies of GH, PRL, and TSH, patients with mutations in the Prop-1 gene may lack leutenizing hormone (LH) and follicle stimulating hormone (FSH) (Procter et al., 1998, Hum. Genet. 103:255-272). Much effort continues to be devoted to the goal of characterizing additional genetic lesions associated with human pituitary disease.

During pituitary development, the actions of the LIM homeodomain transcription factors Lhx3, Lhx4, and Isl-1 are essential for the establishment of Rathke's pouch and the subsequent differentiation of specialized hormone-secreting cell types. LIM homeodomain proteins contain two amino-terminal LIM motifs and interact with DNA using a characteristic homeodomain. The LIM domain is a conserved, zinc finger-like structure that mediates interactions with other proteins, and LIM homeodomain proteins have been demonstrated to be critical to many developmental pathways (as reviewed in Dawid et al., 1998, Trends Genet. 14:295-306; Jurata and Gill, 1998, Curr. Top. Microbiol. Immunol. 228:75-113; Curtiss and Heilig, 1998, Bioessays 20:58-6910-12).

During early embryogenesis, the Lhx3 gene is expressed in several regions of the brain and spinal cord, and then it becomes restricted to the primordial pituitary cells of Rathke's pouch and their descendents in the adult gland (Taira et al., 1993, Dev. Biol. 159:245-256; Seidah et al., 1994, DNA Cell Biol. 13:1163-1180; Bach et al., 1995, Proc. Natl. Acad. Sci. USA 92:2720-2724; Zhadanov et al., 1995, Dev. Dynam. 202:354-364; Glasgow et al., 1997, Dev. Biol. 192:405-419). In the nervous system, the Lhx3, Lhx4, and Isl-1 gene products are required for the specification of motor neurons that emerge from the developing neural tube (e.g., Thor et al., 1999, Nature 397:76-80; Tsuchida et al., 1994, Cell 79:957-970; Sharma et al., 1998, Cell 95:817-828; Osumi et al., 1997, Development 124:2961-2972).

Elegant studies of mice with ablated Lhx3 genes have demonstrated the importance of Lhx3 in pituitary development. That is, pituitary development is arrested in this art-recognized model of pituitary development after the formation of Rathke's pouch and that Lhx3 is required for differentiation of the hormone-secreting cells of the pituitary (Sheng et al., 1996, Science 272:1004-1007; Sheng et al., 1997, Science 278:1809-1812). Lhx4 also is required for complete development of Rathke's pouch; but unlike Lhx3, this factor is not essential for the determination and specification of differentiated pituitary cell types (Sheng et al., 1997, Science 278:1809-1812). Mice lacking both Lhx3 and Lhx4 genes do not develop a rudimentary Rathke's pouch, indicating that at least one of these genes is required during the initial stages of pituitary development (Sheng et al., 1997, Science 278:1809-1812). Further, Lhx3 can activate pituitary trophic hormone genes, acting both alone and with other pituitary transcription factors such as Pit-1 and Pitx1/P-Otx (Bach et al., 1995, Proc. Natl. Acad. Sci. USA 92:2720-2724; Bach et al., 1997, Genes Dev. 11:1370-1380; Meier et al., 1999, Mol. Cell. Endocrinol. 147:65-74).

Although the prior art indicated that Lhx3 plays in important role in pituitary development and functioning in mammals, additional functional analyses are required to fully understand the role of products of the Lhx3 gene in the developing nervous system, the early establishment of Rathke's pouch, and in later pituitary gland function and maintenance have not been possible since the human gene has not been isolated and characterized. Thus, there is a long-felt need to identify and characterize Lhx3 in order that diseases, disorders, and conditions involving the pituitary may be better understood. Lhx3 provides a means for the development of therapeutic and diagnostic tools related to pituitary disease.

### BRIEF SUMMARY OF THE INVENTION

The invention includes an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11.

In one aspect, the nucleic acid sequence of the nucleic acid is SEQ ID NO:1.

The invention further includes an isolated nucleic acid encoding a porcine Lhx3, the nucleic acid having at least about 88% identity with a nucleic acid selected from the group consisting of SEQ ID NO:1, SEQ ID NO:13, and SEQ ID NO:15.

The invention also includes an isolated nucleic acid encoding a human Lhx3, the nucleic acid having at least about 88.5% identity with a nucleic acid having the nucleotide sequence of at least one of SEQ ID NO:7, SEQ ID NO:9, and SEQ ID NO:11.

In one aspect, the isolated nucleic acid further comprises a nucleic acid encoding a tag polypeptide covalently linked thereto.

In another aspect, the tag polypeptide is selected from the group consisting of a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, and a maltose binding protein tag polypeptide.

In yet another aspect, the nucleic acid further comprises a nucleic acid encoding a promoter/regulatory sequence operably linked thereto.

The invention includes a vector comprising an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11.

The invention further includes a recombinant cell comprising an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11.

The invention also includes an isolated polypeptide encoded by the an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11.

In one aspect, the isolated polypeptide shares at least about 93% identity with a polypeptide having the amino acid sequence SEQ ID NO:2.

In another aspect, the polypeptide shares at least about 94.5% identity with a polypeptide having the amino acid sequence of at least one of SEQ ID NO:8, SEQ ID NO:10, and SEQ ID NO:12.

The invention includes ann antibody that specifically binds with the isolated polypeptide of claim 10, or a fragment thereof.

In one aspect, the antibody specifically binds with a portion of the isolated polypeptide selected from the group consisting of a portion from about amino acid residue 1 to about amino acid residue 26 of SEQ ID NO:10, a portion from about amino acid residue 1 to about amino acid residue 31 of SEQ ID NO:12, a portion from about amino acid residue I to about amino acid residue 29 of SEQ ID NO:14, and a portion from about amino acid residue 1 to about amino acid residue 31 of SEQ ID NO:16.

In yet another aspect, the antibody is selected from the group consisting of a polyclonal antibody, a monoclonal antibody, and a synthetic antibody.

The invention includes a composition comprising an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11, and a pharmaceutically-acceptable carrier.

The invention includes a composition comprising an isolated polypeptide encoded by an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11, and a pharmaceutically-acceptable carrier.

The invention includes an isolated nucleic acid encoding a mammalian Lhx3, wherein the nucleic acid specifically hybridizes under high stringency conditions to a nucleic acid selected from the group consisting of a nucleic acid having the nucleic acid sequence SEQ ID NO:1, a nucleic acid having the nucleic acid sequence SEQ ID NO:7, a nucleic acid having the nucleic acid sequence SEQ ID NO:9, a nucleic acid having the nucleic acid sequence SEQ ID NO:11, a nucleic acid having the nucleic acid sequence SEQ ID NO:13, and a nucleic acid having the nucleic acid sequence SEQ ID NO:15, or a nucleic acid complementary to any one of SEQ ID NO:1, SEQ ID NO:7, SEQ ID NO:9, SEQ ID NO:11, SEQ ID NO:13, and SEQ ID NO:15.

The invention also includes an isolated nucleic acid having at least about 45% identity with from about nucleotide −104 to about nucleotide 78 of SEQ ID NO:9.

The invention includes an isolated nucleic acid having at least about 67% identity with from about nucleotide −119 to about nucleotide 93 of SEQ ID NO:11.

The invention includes a method for detecting the presence or absence of a nucleic acid encoding a mammalian Lhx3 having at least about 88.5% homology with a nucleic acid having the sequence SEQ ID NO:7 in a sample. The method comprises

(a) contacting a sample with a nucleic acid probe or primer which specifically hybridizes with the nucleic acid; and

(b) determining whether the nucleic acid probe or primer binds with a nucleic acid in the sample whereby when the nucleic acid probe or primer binds with a nucleic acid in the sample, the sample contains a nucleic acid which specifically hybridizes with the nucleic acid, thereby detecting the presence or absence of the nucleic acid in a sample.

In one aspect, the sample comprises mRNA molecules.

The invention includes a method for detecting the presence or absence of a nucleic acid encoding a mammalian Lhx3 in a sample. The method comprises

(a) amplifying a nucleic acid encoding a mammalian Lhx3 present in a sample wherein the nucleic acid shares about 88.5% identity with a nucleic acid sequence selected from the group consisting of SEQ ID NO:1, SEQ ID NO:7, SEQ ID NO:9, SEQ ID NO:11, SEQ ID NO:13, and SEQ ID NO:15;

(b) detecting any amplified target nucleic acid formed in (a) whereby when the nucleic acid is amplified the sample contains the nucleic acid which encodes a mammalian Lhx3, thereby detecting the presence or absence of a nucleic acid encoding a mammalian Lhx3 in a sample.

The invention includes a method of quantitatively detecting a nucleic acid encoding a mammalian Lhx3 in a sample. The method comprises

(a) contacting a target nucleic acid encoding a mammalian Lhx3 present in a sample with polymerase chain reaction reagents specific for the target nucleic acid, the polymerase chain reaction reagents including at least two polymerase chain reaction primers, a polymerase enzyme, and an internal fluorescent oligonucleotide probe. The oligonucleotide probe further comprises:

(i) a sequence capable of hybridizing to a portion of the target nucleic acid;

(ii) a fluorescer molecule attached to the 3′ end of the oligonucleotide;

(iii) a quencher molecule attached to the 5′ end of the oligonucleotide capable of substantially quenching the fluorescer molecule when the oligonucleotide is intact, wherein the fluorescer molecule becomes substantially unquenched when the oligonucleotide probe is cleaved by DNA polymerase during amplification of the target nucleic acid; and

(iv) the 3′ end is impervious to the 5′→3′ extension activity of the polymerase enzyme; and

(b) amplifying the target nucleic acid by thermal cycling, wherein the thermal cycling is sufficient to amplify the target nucleic acid; and

(c) measuring the level of fluorescence in the sample subsequent to thermal cycling, and further wherein the level of fluorescence is correlated to the amount of target nucleic acid present in the sample, thereby quantitatively detecting a target nucleic acid encoding a mammalian Lhx3 in a sample.

In one aspect, the polymerase chain reaction primer is selected from the group consisting of a nucleic acid having the sequence SEQ ID NO:44 (ggcacgagccccgcacgacg-3′), and a nucleic acid having the sequence of SEQ ID NO:45 (5′-tttgaagtcttggaaagtgc-3′).

The invention includes a kit comprising a compound which specifically hybridizes with a nucleic acid having at least about 88.5% homology to a nucleic acid having the sequence of at least one of SEQ ID NO:7, SEQ ID NO:9, and SEQ ID NO:11, and an instructional material for the use thereof.

In one aspect, the compound is selected from the group consisting of a nucleic acid and an antibody.

In another aspect, the nucleic acid is selected from the group consisting of a polymerase chain reaction primer, and an internal oligonucleotide probe.

The invention includes a kit for detecting a nucleic acid encoding a mammalian Lhx3, the kit comprising a first nucleic acid having the sequence selected from the group consisting of SEQ ID NO:34 (5′-atgctgctggaaacggggctcg-3′), and SEQ ID NO:36 (5′-atggaggcgcgcggggagct-3′), and a second nucleic acid having the sequence selected from the group consisting of SEQ ID NO:35 (5′-ccgagtcccgcccaaggtgc-3′), and SEQ ID NO:37 (5′-ctcggcgcaggtctgccctc-3′), and an instructional material for the use thereof.

In one aspect, the kit further comprises an internal oligonucleotide probe complementary to at least a portion of a nucleic acid having the sequence of from about nucleotide I to about nucleotide 8867 of SEQ ID NO:22.

In another aspect, the oligonucleotide probe has the sequence selected from the group consisting of SEQ ID NO:38 (5′-gcgaccgagcgaggcccggggccgc-3′), SEQ ID NO:39 (5′-cccggcccgggagtcggcgggaggc-3′), and SEQ ID NO:40 (5′-ttcccgatgagccttccttggcggaa-3′).

The invention includes a method of quantitatively detecting a nucleic acid encoding a mammalian Lhx3a in a sample. The method comprises

(a) contacting a target nucleic acid encoding a mammalian Lhx3a present in a sample with polymerase chain reaction reagents specific for the target nucleic acid, the polymerase chain reaction reagents including at least two polymerase chain reaction primers, a polymerase enzyme, and an internal fluorescent oligonucleotide probe, the oligonucleotide probe further comprising:

(i) a sequence capable of hybridizing to a portion of the target nucleic acid wherein the portion is unique to Lhx3a;

(ii) a fluorescer molecule attached to a 5′ end of the oligonucleotide;

(iii) a quencher molecule attached to a 3′ end of the oligonucleotide capable of substantially quenching the fluorescer molecule when the oligonucleotide is intact, wherein the fluorescer molecule becomes substantially unquenched when the oligonucleotide probe is cleaved by DNA polymerase during amplification of the target nucleic acid; and

(iv) a 3′ end which is impervious to the 5′→3′ extension activity of the DNA polymerase; and

(b) amplifying the target nucleic acid by thermal cycling, wherein the thermal cycling is sufficient to amplify the target nucleic acid; and

(c) measuring the level of fluorescence in the sample subsequent to thermal cycling, and further wherein the level of fluorescence is correlated to the amount of target nucleic acid present in the sample, thereby quantitatively detecting a target nucleic acid encoding a mammalian Lhx3a in a sample.

In one aspect, the polymerase chain reaction primer is selected from the group consisting of a first nucleic acid having a sequence selected from the group consisting of SEQ ID NO:34, and a second nucleic acid having a sequence selected from the group consisting of SEQ ID NO:35.

The invention includes a kit for quantitatively detecting a nucleic acid encoding a mammalian Lhx3a, the kit comprising a polymerase chain reaction primer having the sequence selected from the group consisting of SEQ ID NO:34, and SEQ ID NO:35, and an instructional material for the use thereof.

In one aspect, the kit further comprises an internal oligonucleotide probe which specifically hybridizes with a nucleic acid molecule comprising a nucleic acid having the sequence of SEQ ID NO:38.

In further aspect, the internal oligonucleotide probe has the sequence SEQ ID NO:40.

The invention also includes a kit for quantitatively detecting a nucleic acid encoding a mammalian Lhx3b. The kit comprises a polymerase chain reaction primer having the sequence selected from the group consisting of SEQ ID NO:36, and SEQ ID NO:37, and an instructional material for the use thereof.

In one aspect, the kit further comprises an internal oligonucleotide probe which specifically hybridizes with a nucleic acid molecule having the sequence of SEQ ID NO:39.

The invention includes a method of quantifying the level of Lhx3 expressed in a cell. The method comprises reverse transcribing any ribonucleic acid encoding Lhx3 present in a cell, amplifying any deoxyribonucleic acid encoding Lhx3 produced by reverse transcription, and detecting any amplified deoxyribonucleic acid formed using quantitative sequence detection, thereby quantifying the level of Lhx3 expressed in a cell.

In one aspect, the Lhx3 is selected from the group consisting of porcine Lhx3a, porcine Lhx3b, human Lhx3a, and human Lhx3b.

In a further aspect, the amplification comprises amplification using polymerase chain reaction wherein the polymerase chain reaction comprises a primer having a sequence selected from the group consisting of SEQ ID NO:41 (5′-ggcacgagccccgcacgacg-3′), and SEQ ID NO:35.

In one aspect, the detection comprises contacting any deoxyribonucleic acid produced with an internal fluorescent oligonucleotide probe which specifically hybridizes with a nucleic acid an isolated nucleic acid encoding a mammalian Lhx3. The nucleic acid is selected from the group consisting of an isolated nucleic having at least about 88% identity with at least one of SEQ ID NO:1, SEQ ID NO:13, and SEQ ID NO:15. Further, the isolated nucleic acid has at least about 88.5% identify with at least one of SEQ ID NO:7, SEQ ID NO:9, and SEQ ID NO:11.

The invention includes a kit for detecting a nucleic acid encoding a mammalian Lhx3a, the kit comprising a first nucleic acid having the sequence SEQ ID NO:34, and a second nucleic acid having the sequence SEQ ID NO:35, and an instructional material for the use thereof.

In one aspect, the kit further comprises an internal oligonucleotide probe complementary to at least a portion of a nucleic acid having the sequence of SEQ ID NO:9.

The invention includes a kit for detecting a nucleic acid encoding a mammalian Lhx3b, the kit comprising a first nucleic acid having the sequence SEQ ID NO:36, and a second nucleic acid having the sequence SEQ ID NO:37, and an instructional material for the use thereof.

In one aspect, the kit further comprises an internal oligonucleotide probe complementary to at least a portion of a nucleic acid having the sequence of SEQ ID NO:11.

In yet a further aspect, the oligonucleotide probe has the sequence SEQ ID NO:40.

The invention also includes a method of identifying a compound that affects expression of human Lhx3 in a cell, the method comprising contacting a cell with a compound and comparing the level of expression of Lhx3 in the cell contacted with the compound with the level of expression of Lhx3 is an otherwise identical cell, wherein a higher or lower level of expression of Lhx3 is the cell contacted with the compound compared with the level of expression of Lhx3 in the otherwise identical cell not contacted with the compound, is an indication that the compound affects expression of Lhx3 in a cell.

In one aspect, the human Lhx3 is selected from the group consisting of hLhx3a, and hLhx3b.

The invention includes a method of identifying a compound that affects the level of expression of Lhx3a but not the level of expression of Lhx3b in a cell. The method comprises contacting a cell with a compound and comparing the level of expression of Lhx3a in a cell with the level of expression of Lhx3a in an otherwise identical cell not contacted with the compound, and further comparing the level of expression of Lhx3b in the cell contacted with the compound with the level of expression of Lhx3b in the otherwise identical not contacted with the compound, wherein a higher or lower level of expression of Lhx3a in the cell contacted with the compound compared with the level of expression of Lhx3a in the otherwise identical cell not contacted with the compound, and further wherein there is no detectable change in the level of expression of Lhx3b in the cell contacted with the compound compared to the level of expression of Lhx3b in the otherwise identical cell not contacted with the compound, is an indication that the compound affects the level of expression of Lhx3a but not the level of expression of Lhx3b in a cell.

The invention further includes a method of identifying a compound that affects the level of expression of Lhx3b but not the level of expression of Lhx3a in a cell. The method comprises contacting a cell with a compound and comparing the level of expression of Lhx3b in a cell with the level of expression of Lhx3b in an otherwise identical cell not contacted with the compound, and further comparing the level of expression of Lhx3a in the cell contacted with the compound with the level of expression of Lhx3a in the otherwise identical not contacted with the compound, wherein a higher or lower level of expression of Lhx3b in the cell contacted with the compound compared with the level of expression of Lhx3b in the otherwise identical cell not contacted with the compound, and further wherein there is no detectable change in the level of expression of Lhx3a in the cell contacted with the compound compared to the level of expression of Lhx3a in the otherwise identical cell not contacted with the compound, is an indication that the compound affects the level of expression of Lhx3b but not the level of expression of Lhx3a in a cell.

The invention includes a method of identifying a compound which affects the activity of human Lhx3 in a cell. The method comprises contacting a cell with a compound and comparing the level of activity of Lhx3 in the cell contacted with the compound with the level of activity of Lhx3 in an otherwise identical cell not contacted with the compound, wherein a higher or lower level of activity of Lhx3 is the cell contacted with the compound compared with the level of activity of Lhx3 in the otherwise identical cell not contacted with the compound, is an indication that the compound affects the activity of Lhx3 in a cell.

In one aspect, the activity of the human Lhx3 is selected from the group consisting of the ability of hLhx3 to induce expression of a reporter gene operably linked to a αGSU promoter, the ability of hLhx3 to induce expression of a reporter gene operably linked to a TSHβ promoter, the ability of hLhx3 to bind to the −350 to −323 bp region of murine αGSU promoter, the ability to activate the gene encoding Pit-1, and the ability to activate the gene encoding prolactin.

In a further aspect, the human Lhx3 is selected from the group consisting of Lhx3a, and Lhx3b.

The invention includes a method of identifying a compound which affects the level of activity of Lhx3 in a cell. The method comprises

(a) transfecting a cell with a vector encoding Lhx3;

(b) co-transfecting the cell transfected with a reporter gene construct wherein the construct comprises a reporter gene operably linked to a promoter/regulatory sequence that is trans-activated by Lhx3;

(c) contacting the cell of (b) with a test compound;

(d) measuring the level of activity of Lhx3 in the cell of (b) before and after contacting the cell with the compound;

(f) comparing the level of activity of Lhx3 in the cell contacted with the compound with the level of activity of Lhx3 in the cell prior to or in the absence of contacting the cell with the compound. A higher or lower level of activity of Lhx3 in the cell contacted with the compound compared with the level of activity of Lhx3 in the cell prior to or in the absence of contacting the cell with the compound is an indication that the compound affects the level of activity of Lhx3 in a cell.

The invention includes a method of identifying a compound which affects the level of activity of Lhx3a but not the level of activity of Lhx3b in a cell. The method comprises

(a) transfecting a cell having no detectable endogenous Lhx3a activity with an expression vector encoding Lhx3a;

(b) transfecting an otherwise identical cell the cell having no detectable endogenous Lhx3b activity with an expression vector encoding Lhx3b;

(c) co-transfecting each of the cells in (a) and (b) with a reporter gene construct comprising a reporter gene operably linked to a promoter/regulatory sequence trans-activated by Lhx3a and Lhx3b;

(d) contacting each of the cells of (c) with a test compound;

(e) measuring the level of Lhx3a activity in each of the cells of (c) before and after contacting each of the cells with the test compound;

(f) measuring the level of Lhx3b activity in each of the cells of (c) before and after contacting each of the cells with the test compound;

(g) comparing the level of activity of Lhx3a in each of the cells of (c) contacted with the compound to the level of activity of Lhx3a in each of the cells prior to or in the absence of contacting each of the cells with the test compound;

(h) comparing the level of activity of Lhx3b in each of the cells of (c) contacted with the test compound to the level of activity of Lhx3b in each of the cells prior to or in the absence of contacting each of the cells with the test compound. A wherein a higher or lower level of activity Lhx3a in each of the cells of (c) contacted with the test compound compared to the level of activity of Lhx3a in each of the cells prior to or in the absence of contacting each of the cells with the test compound and further wherein there is no detectable change in the level of Lhx3b activity in each of the cells of (c) contacted with the test compound compared to the level of Lhx3b activity in each of the cells of (c) prior to or in the absence of contacting each of the cells with the test compound, is an indication that the test compound affects the level of Lhx3a activity but not the level of Lhx3b activity in a cell.

The invention includes a method of identifying a compound which affects binding of Lhx3 to a nucleic acid that specifically binds with Lhx3. The method comprises administering a compound into an extract wherein the extract comprises Lhx3 and a nucleic acid that specifically binds with Lhx3, and comparing the level of binding of Lhx3 with the nucleic acid in the extract comprising the compound with the level of binding of Lhx3 with the nucleic acid in an otherwise identical extract which does not comprise the compound, wherein a higher or lower level of binding of Lhx3 with the nucleic acid in the extract comprising the compound compared to the level of binding of Lhx3 to the nucleic acid in the otherwise identical extract not comprising the compound, is an indication that the compound affects binding of Lhx3 with a nucleic acid that specifically binds with Lhx3.

In one aspect, the Lhx3 is selected from the group consisting of human Lhx3a, human Lhx3b, porcine Lhx3a and porcine Lhx3b.

In a further aspect, the nucleic acid that specifically binds with Lhx3 is selected from the group consisting of a nucleic acid having nucleotides −350 to −323 of murine αGSU promoter (5′-gatccggtacttagctaattaaatga-3′) [SEQ ID NO:42]), and a nucleic acid encoding the Lhx3 consensus binding sequence (5′-gatcccagaaaattaattaattgtaa-3′) [SEQ ID NO:43]).

The invention includes a method of identifying a compound that affects Lhx3 induction of a pituitary trophic hormone gene promoter. The method comprises

(a) transfecting a cell with a reporter gene construct wherein the construct comprises a thyroid-stimulating hormone beta promoter sequence operably linked to a reporter gene;

(b) transfecting the cell of (a) with a hLhx3 expression vector;

(c) contacting the cell of (b) with a pituitary transcription factor which synergizes with Lhx3;

(d) contacting the cell of (c) with a compound;

(e) assessing the level of expression of the reporter gene where the level of expression of the reporter gene is correlated to the level of Lhx3 induction of the promoter; and

(f) comparing the level of expression of the reporter gene in the cell contacted with the compound with the level of expression of the reporter gene in an otherwise identical cell not contacted with the compound,

wherein in a higher or lower level of expression of the reporter gene in the cell contacted with the compound compared with the level of expression of the reporter gene in the otherwise identical cell not contacted with the compound, is an indication that the compound affects Lhx3 induction of a pituitary trophic hormone gene promoter in a cell.

In one aspect, the pituitary trophic hormone gene promoter is selected from the group consisting of a thyroid-stimulating hormone beta promoter, an alpha-glycoprotein subunit promoter, and a prolactin promoter.

In a further aspect, the pituitary transcription factor which synergizes with Lhx3 is selected from the group consisting of Pit-1, Pitx1/P-Otx, and thyrotrope embryonic factor.

In yet another aspect, the Lhx3 is selected from the group consisting of Lhx3a, Lhx3b, pLhx3a, and pLhx3b.

The invention further includes a method of identifying a human patient afflicted with a disease, disorder or condition associated with altered expression of Lhx3. The method comprises detecting the level of Lhx3 expression in a human and comparing the level of expression of Lhx3 in the human with the level of expression of Lhx3 in a normal human not afflicted with a disease, disorder or condition associated with altered expression of Lhx3, thereby detecting a human patient afflicted with a disease, disorder or condition associated with altered expression of Lhx3.

The invention includes a method of identifying a human patient afflicted with a disease, disorder or condition associated with altered level of binding of Lhx3 to a nucleic acid that specifically binds with Lhx3. The method comprises detecting the level of Lhx3 binding with a nucleic acid that specifically binds with Lhx3 in a human and comparing the level of Lhx3 binding with a nucleic acid that specifically binds with Lhx3 in the human with the level of Lhx3 binding with a nucleic acid that specifically binds with Lhx3 in a normal human not afflicted with a disease, disorder or condition associated with altered level of Lhx3 binding with a nucleic acid that specifically binds with Lhx3, wherein a higher or lower level of Lhx3 binding with a nucleic acid that specifically binds with Lhx3 in the human compared with the level of Lhx3 binding with a nucleic acid that specifically binds with Lhx3 in the human not afflicted with a disease, disorder or condition associated with altered level of Lhx3 binding with a nucleic acid that specifically binds with Lhx3, is an indication that the human is afflicted with a disease, disorder or condition associated with altered level of Lhx3 binding with a nucleic acid that specifically binds with Lhx3, thereby detecting a human patient afflicted with a disease, disorder or condition associated with altered level of Lhx3 binding with a nucleic acid that specifically binds with Lhx3.

In one aspect, the nucleic acid that specifically binds with Lhx3 is selected from the group consisting of nucleotides −350 to −323 of murine αGSU promoter, and the Lhx3 consensus binding sequence.

The invention also includes a method of detecting a mutation in a Lhx3 allele in a human. The method comprises comparing the nucleic acid sequence encoding Lhx3 of a human suspected of having a mutation in Lhx3 with the nucleic acid sequence encoding Lhx3 obtained from a normal human not having a mutation in Lhx3, wherein any difference between the nucleic acid sequence of the human suspected of having a mutation in Lhx3 and the nucleic acid sequence encoding Lhx3 of the normal human not having a mutation in Lhx3 detects a mutation in a Lhx3 allele in the human.

The invention includes a method of detecting a mutation in a Lhx3 allele in a human. The method comprises comparing the genomic nucleic acid sequence encoding Lhx3 of a human suspected of having a mutation in Lhx3 with the genomic nucleic acid sequence encoding Lhx3 obtained from a normal human not having a mutation in Lhx3, wherein any difference between the genomic nucleic acid sequence of the human suspected of having a mutation in Lhx3 and the genomic nucleic acid sequence encoding Lhx3 of the normal human not having a mutation in Lhx3 detects a mutation in a Lhx3 allele in the human.

### BRIEF DESCRIPTION OF THE DRAWINGS

The foregoing summary, as well as the following detailed description of the invention, will be better understood when read in conjunction with the appended drawings. For the purpose of illustrating the invention there are shown in the drawings embodiment(s) which are presently preferred. It should be understood, however, that the invention is not limited to the precise arrangements and instrumentalities shown.

In the drawings:

FIG. 1 is a diagram depicting the predicted amino acid sequence of porcine Lhx3 [SEQ ID NO:2] and comparison to murine P-Lim/Lhx3/LIM-3 (now referred to herein as mLhx3a) as described in Bach et al. (1995, Proc. Natl. Acad. Sci. USA 92:2720-724) (M. GenBank Accession Number 780314) [SEQ ID NO:3], chicken LIM-3 (C. GenBank Acc. No. 1708828 [SEQ ID NO:4]), Xenopus lim-3 (X, GenBank Acc. No. 547856 [SEQ ID NO:5]) and zebrafish LIM-3 (Z, GenBank Acc. No. 2497671 [SEQ ID NO:6]). Alignment is based on that of Glasgow et al. (1997, Dev. Biol. 192:405-419) for zebrafish LIM-3. The LIM domains and homeodomain are bracketed. Dashes indicate identity and asterisks denote gaps introduced to optimize alignment. The GenBank accession number for the porcine Lhx3 nucleotide sequence is AF063245.

FIG. 2 is an image depicting a Northern analysis of Lhx3 messenger RNA in porcine pituitary. RNA obtained from the indicated tissues was separated on denaturing gels, transferred to nylon membranes and probed sequentially using radiolabeled cDNA probes. A blot containing 30 μg of RNA per lane was used for the Lhx3 and prolactin probes; the Pit-1 and growth hormone blot contained 15 μg per lane. The migration positions of ribosomal RNAs are indicated on the left side of the image. An image of an ethidium bromide stained gel depicting ribosomal RNAs for the Lhx3/prolactin lanes used for Northern blotting is depicted at the bottom of the figure to demonstrate lane loading.

FIG. 3A, comprising FIGS. 3Ai and 3Aii, is a diagram depicting activation of alpha-glycoprotein subunit (αGSU) gene promoter luciferase plasmids, the structure of which is depicted at the top of the figure, by pLhx3. Briefly, 293 cells were transiently transfected with porcine αGSU reporter gene construct and the indicated vectors (i.e., control vector without insert, pLhx3 vector, and ΔLIM pLhx3 vector). Promoter activity was assessed by measurement of luciferase activity after 48 hours. Luciferase activities disclosed are mean [light units/10 seconds/μg total protein] of triplicate assays ±S.E.M. Representative experiments of at least nine experiments are depicted.

FIG. 3B, comprising FIGS. 3Bi and 3Bii, is a diagram depicting activation of alpha-glycoprotein subunit (αGSU) gene promoter luciferase plasmids, the structure of which is depicted at the top of the figure, by pLhx3. Briefly, 293 cells were transiently transfected with mouse αGSU reporter gene construct and the indicated vectors (i.e., control vector without insert, pLhx3 vector, and ΔLIM pLhx3 vector). Alpha-GSU promoter activity was assayed by measurement of luciferase activity after 48 hours. Luciferase activities are mean [light units/10 seconds/μg total protein] of triplicate assays ±S.E.M. Representative experiments of at least twelve experiments are depicted.

FIG. 3C is an image of a Western blot depicting expression of pLhx3 protein of predicted molecular weight in 293 cells transfected with myc-epitope-tagged pLhx3 expression vectors but not in cells transfected with control vector. The protein was detected using an anti-myc monoclonal antibody.

FIG. 4A is an image depicting expression of recombinant porcine pLhx3 proteins using SDS-polyacrylamide gel analysis of GST fusion proteins containing either pLhx3 (GST-pLhx3) or ΔLIM pLhx3 (GST-ΔLIM). An image of a Coomassie blue stain of a protein gel is depicted. The migration of molecular weight standards is indicated (i.e., 69 kDa and 46 kDa).

FIG. 4B is an image depicting an analysis of recombinant porcine pLhx3 protein binding to DNA target sequences. The image depicts electrophoretic mobility shift assay using labeled wild type and mutant oligonucleotides representing the −351 to −324 bp region of the porcine αGSU gene promoter. Probes were incubated with the indicated proteins and competitor DNA and the resulting complexes were separated from free probe (F) by electrophoresis. The panel on the right of the figure was exposed three times as long as the left-hand panel to detect weak binding of GST-pLhx3 to the porcine αGSU binding site (which is indicated by an arrow).

FIG. 5, comprising FIGS. 5A and 5B, is a diagram depicting synergistic activation of a prolactin enhancer/promoter reporter gene by Pit-1 and pLhx3 or ΔLIM pLhx3. Briefly, 293 cells were transiently transfected with a rat prolactin enhancer/promoter reporter gene plasmid depicted at the top of the figure, and the indicated expression vectors. Promoter activity was assayed by measurement of luciferase activity after 48 hours. Activities are mean [light units/10 seconds/μg total protein] of triplicate assays ±S.E.M. A representative experiment of at least six experiments is depicted.

FIG. 6, comprising FIGS. 6A-6C, is a series of images depicting in vitro binding assays demonstrating interaction of pLhx3 with NLI and Pit-1 proteins. Radiolabeled proteins were generated by translation in the presence of [³⁵S-methionine. The radiolabeled protein was incubated with the indicated GST fusion proteins or with excess GST alone as a control. After washing, bound proteins were separated by electrophoresis and were visualized by fluorography. The migration positions of molecular weight standards (in kDa) are depicted at the right of each figure.

FIG. 7, comprising FIGS. 7A and 7B, is a diagram depicting the nucleic acid sequence of porcine Lhx3 (SEQ ID NO:2).

FIG. 8 is a diagram depicting the amino acid sequence of porcine Lhx3 (SEQ ID NO:1).

FIG. 9A is a schematic depiction of the domain structures of hLhx3a and hLhx3b. The hatched regions represent domains unique to each form: L, LIM domain; HD, homeodomain; LSD, Lhx3/LIM3-specific domain.

FIG. 9B is a diagram depicting the amino acid sequences and alignment of the sequences of mammalian Lhx3 proteins. Comparison of human Lhx3a/b (Ha [SEQ ID NO:10], Hb [SEQ ID NO:12]), murine Lhx3a and b as described by Zhadanov et al. (1995, Dev. Dynamics 202:354-364) (Ma, GenBank Acc. No. L38249 [SEQ ID NO:3]; Mb, GenBank Acc. No. L38248 [SEQ ID NO:21]), and porcine Lhx3 (P, AF063245 [SEQ ID NO:1]). The LIM domains (L), homeodomain (HD), and Lhx3/LIM3-specific domain (LSD) are boxed/reversed. Dots indicate identity; dashes denote gaps introduced to optimize alignment.

FIG. 9C is a diagram depicting the similarity of Lhx3/LIM3 family proteins. The numbers indicated above each domain indicate the percentage identity to hLhx3 for that domain. The organisms are abbreviated as follows with the GenBank Accession Numbers provided in parentheses: c, Chicken (GenBank accession number 1708828 [SEQ ID NO:4]); X, Xenopus laevis (GenBank Accession No. 547856 [SEQ ID NO:5]); z, Zebrafish (GenBank Accession No. 2497671 [SEQ ID NO:6]); mLhx4, mouse Lhx4 (GenBank Accession No. AF 135415 [SEQ ID NO:20]); d, Drosophila melanogaster (GenBank Accession No. AF 109306 [SEQ ID NO:21 I]).

FIG. 10, comprising FIGS. 10A, 10B, and 10C, is a diagram depicting the nucleic acid sequence of human Lhx3a (SEQ ID NO:9) cDNA. The start nucleotide is located at 1 nts and is designated at the commencement of upper case. The LIM Domain 1 located at nucleotides 91-223 is underlined as is LIM Domain 2 at nucleotides 268-432. The homeodomain at nucleotides 466-660 is also underlined. The stop nucleotide is located at position 1194.

FIG. 11, comprising FIGS. 11A, 11B, and 11C, is a diagram depicting the nucleic acid sequence of human Lhx3b (SEQ ID NO:11). The start nucleotide at position 1 is designated by upper case letters. The start nucleotide is located at 1 nts and is designated at the commencement of upper case. The LIM Domain 1 located at nucleotides 106-248 is underlined as is LIM Domain 2 at nucleotides 283-447. The homeodomain at nucleotides 481-675 is also underlined. The stop nucleotide is located at position 1209.

FIG. 12A is an image of a Southern blot depicting the fact that hLhx3 is encoded by single gene. Human genomic DNA was digested with EcoRI and blotted to a nylon membrane. The blot was probed with a hLhx3 cDNA probe at high stringency. The migration position of molecular weight standards (kb) is given.

FIG. 12B is an image of a Northern blot depicting analysis of hLhx3 gene expression. Human kidney (lane 2) or pituitary (Pit.; lane 1) total (20 μg) or poly A⁺ RNA (1 μg; lanes 3 and 4) was separated on a denaturing gel, transferred to a nylon membrane, and probed with the indicated radiolabeled cDNA probes (e.g., Lhx3 and Pit-1). The migration positions of ribosomal RNAs (i.e., 28S and 18S) are indicated. The arrowhead indicates hLhx3 RNA.

FIG. 12C is an image of SDS-PAGE analysis of human Lhx3a and hLhx3b proteins. [³⁵S]-methionine radiolabeled hLhx3a and hLhx3b isoforms (lanes 1 and 2) and myc epitope-tagged derivatives (a-myc, b-myc; lanes 4 and 5) were generated by in vitro transcription/translation. The proteins were separated by SDS electrophoresis, and dried gels were visualized using fluorography. The migration positions of protein standards (in kilodaltons) are shown. Lane 3 is control.

FIG. 13A is an image depicting the localization of control green fluorescent protein (GFP) in cells. Human 293T cells were transiently transfected with expression vectors encoding multimerized green fluorescent protein (GFP) as a control (4xGFP). The cells were examined using phase contrast microscopy. Bar=10 μm.

FIG. 13B is an image depicting the localization of control GFP protein using fluorescence microscopy. Human 293T cells were transiently transfected with expression vectors encoding multimerized green fluorescent protein (GFP) as a control (4xGFP). The fluorescence was visualized using krypton-argon laser scanning confocal microscopy. 4xGFP control is restricted to the cytoplasm. Bar=10 μm.

FIG. 13C is an image depicting the localization of hLhx3a-GFP in cells. Human 293T cells were transiently transfected with expression vectors encoding hLhx3a-4xGFP. The cells were examined using phase contrast microscopy to visualize cell structures. Unlike control 4xGFP, which is restricted to the cytoplasm, hLhx3a4xGFP is detected in the nuclei of transfected cells. Bar=10 μm.

FIG. 13D is an image depicting the localization of hLhx3a-GFP using fluorescence microscopy. Human 293T cells were transiently transfected with expression vectors encoding hLhx3a4xGFP. The fluorescence was detected using krypton-argon laser scanning confocal microscopy. Unlike 4xGFP control, which is restricted to the cytoplasm, hLhx3a-4xGFP is detected in the nuclei of transfected cells. Bar=10 μm.

FIG. 13E is an image depicting the localization of hLhx3b-4xGFP in cells. Human 293T cells were transiently transfected with expression vectors encoding hLhx3b-4xGFP. The cells were examined using phase contrast microscopy to visualize cell structures. Unlike control 4xGFP, which is restricted to the cytoplasm, hLhx3b-4xGFP is detected in the nuclei of transfected cells. Bar=10 μm.

FIG. 13F is an image depicting the localization of hLhx3b-4xGFP using fluorescence microscopy. Human 293T cells were transiently transfected with expression vectors encoding hLhx3b-4xGFP. The fluorescence was detected using krypton-argon laser scanning confocal microscopy. Unlike 4xGFP control, which is restricted to the cytoplasm, hLhx3b-4xGFP is detected in the nuclei of transfected cells. Bar=10 μm.

FIG. 14A, comprising FIGS. 14Ai and 14Aii, is a graph depicting a quantitative fluorescent assay for human Lhx3 gene expression. RNA was extracted from adult human pituitaries and cDNA was obtained using a 3′ gene-specific primer as described elsewhere herein. PCR was performed using gene-specific primers and the PCR reactions were monitored in “real time” using an internal fluorescent hLhx3-specific probe (i.e., a TaqMan probe as described elsewhere herein). The results are the means of four independent experiments ±SEM. The inset graph depicts a standard curve prepared using hLhx3 cDNA as input. In the inset graph, “Ct” denotes the PCR cycle number where the reporter dye fluorescent emission increases above baseline emission as described in Heid et al. (1996, Genome Res. 6:986-994).

FIG. 14B is an image of a gel depicting a RT-PCR analysis of expression of transcripts of the hLhx3 gene encoding hLhx3a and hLhx3b isoforms. RT-PCR was used to amplify the approximately 1.2-kb coding regions of hLhx3a and hLhx3b using pituitary gland cDNA from two representative patients. Reaction products were separated by agarose gel electrophoresis and the gels were stained using ethidium bromide to visualize any RT-PCR amplicons. In the figure, the abbreviations used are as follows: M, molecular weight markers; Control, negative control.

FIG. 14C is an image of a gel depicting the expression of Lhx3 isoforms in pituitary cell lines. RT-PCR was used to amplify specific regions of Lhx3a (139-bp product designated “a”) and Lhx3b (165-bp product designated “b”) using cDNA from the indicated cell lines. Amplification reaction products were separated by acrylamide gel electrophoresis and visualized by staining the gels using ethidium bromide. Negative control reactions (−) were performed in parallel in the absence of reverse transcriptase.

FIG. 15A, comprising FIGS. 15Ai and 15Aii, is a graph depicting differential activation of the alpha-glycoprotein subunit (αGSU) gene promoter by hLhx3 isoforms. Human 293 cells were transiently transfected with a mouse αGSU luciferase reporter gene (schematically diagramed along the top edge of the figure) and the indicated expression vector, i.e., control vector without insert (control), vector comprising an insert encoding hLhx3a (hLhx3a), vector comprising an insert encoding hLhx3a tagged with a myc-epitope (hLhx3a-myc), vector comprising an insert encoding hLhx3b (hLhx3b), and vector comprising an insert encoding hLhx3b tagged with a myc-epitope (hLhx3b-myc). Promoter activity was assayed by measurement of luciferase activity 48 hours post-transfection. Luciferase activities are the mean [light units/10 sec/μg total protein] of triplicate assays ±SEM. A representative experiment of at least seven experiments is depicted.

FIG. 15B is an image depicting a Western blot analysis using an anti-myc monoclonal antibody of cells transfected with control and myc epitope-tagged hLhx3 expression vectors demonstrating expression of hLhx3a and hLhx3b proteins in the cells. The migration positions of protein standards (in kilodaltons) are shown.

FIG. 16A, comprising FIGS. 16Ai and 16Aii, is a diagram depicting the induction of expression of a reporter gene construct containing the TSHβ promoter by hLhx3 isoforms and Pit-1 pituitary transcriptions factor. Human 293 cells were transiently transfected with a mouse TSHβ promoter reporter gene plasmid (as shown along the top edge of the figure) and hLhx3a. hLhx3b, and/or Pit-1 expression vectors. Luciferase activity was assayed 48 hours post-transfection. Values are the mean light units/10 sec/μg total protein] of triplicate assays ±SEM. A representative experiment of at least four experiments is depicted.

FIG. 16B is a diagram depicting the induction of expression of a reporter gene construct containing the TSHβ promoter by hLhx3 isoforms and thyrotrope embryonic factor (TEF) pituitary transcriptions factor. Human 293 cells were transiently transfected with a mouse TSHβ promoter reporter gene plasmid and hLhx3a, hLhx3b, and/or TEF expression vectors. Luciferase activity was assayed 48 hours post-transfection. Values are the mean [light units/10 sec/μg total protein] of triplicate assays ±SEM. A representative experiment of at least four experiments is depicted.

FIG. 17, comprising FIGS. 17A and 17B, is a graph depicting the trans-activation of a luciferase reporter gene containing Lhx3 binding sites by hLhx3 isoforms. Human 293 cells were transiently transfected with a Lhx3 reporter gene containing three Lhx3 consensus sites and hLhx3a or hLhx3b expression vectors as depicted along the top edge of the figure. Reporter gene activity was measured 48 hours after transfection. The luciferase activities disclosed are the mean [light units/10 sec/μg total protein] of triplicate assays ±SEM. A representative experiments of at least five experiments is depicted.

FIG. 18A is an image of a gel depicting an analysis of the expression of recombinant hLhx3 proteins and analysis of Lhx3 isoforms binding of DNA target sequences. The image depicts a Coomassie brilliant blue stain of a SDS-polyacrylamide gel analysis of hLhx3a or hLhx3b fusion proteins containing a glutathione-S-transferase (GST) tag epitope. The migration of molecular weight standards is indicated in kilodaltons along the right-hand edge of the figure.

FIG. 18B is an image of a gel depicting an electrophoretic mobility shift assay (EMSA) using Lhx3 consensus binding site (lanes 1-12) or αGSU gene −350 to −323 binding site (lanes 13 and 14) as oligonucleotide probes. Radiolabeled probes were incubated with the indicated proteins and competitor DNAs and the resulting complexes were separated from free probe (F) by electrophoresis. Lane 1, unprogrammed rabbit reticulocyte lysate as a negative control (lysate); lanes 2-5 (hLhx3a; hLhx3b; hLhx3a-myc; and hLhx36-myc, respectively), reactions contained in vitro translated hLhx3 proteins including isoforms comprising a myc tag epitope. A nonspecific band is noted by an asterisk (*). Human Lhx3 protein/DNA complexes are indicated by an arrow. Lane 6 depicts the lack of binding using GST as a negative control; lanes 7-14 (GST-hLhx3a, more GST-hLhx3a, comp plus hLhx3a, GST-hLhx3b; more GST-hLhx3b, comp plus hLhx3b, GST-hLhx3a, and GST-hLhx36, respectively) depict binding of the DNA probe by recombinant hLhx3 proteins expressed in E. coli. “Comp” denotes reactions containing approximately 1000-fold molar excess of unlabeled binding site as a competitor.

FIG. 19, comprising FIGS. 19A, 19B, and 19C, is a diagram depicting the nucleic acid sequence of human Lhx3c (SEQ ID NO:25).

FIG. 20. comprising FIGS. 20A, 20B, and 20C, is a diagram depicting the nucleic acid sequence of human Lhx3d (SEQ ID NO:26).

FIG. 21, comprising FIGS. 20A, 20B, and 20C, is a diagram depicting the nucleic acid sequence of human Lhx3e (SEQ ID NO:27).

FIG. 22A is a diagram depicting the genomic organization of the human LHX3 gene. more specifically, the structure of the LHX3 gene. Exons are represented by boxes and labeled in Roman numerals. Unfilled boxes indicate untranslated regions; stippled boxes denote protein coding exons. Introns are labeled using Arabic numerals. The asterisk denotes the location of a conserved ATTTA motif. PCR primers: introns 1a and 1b: 5′-tgacctcggaggagcgcgtct-3′ (SEQ ID NO:46) and 5′-tcgtccttgcagtaaacgct-3′ (SEQ ID NO:47); intron 2: 5′-agcgtttactgcaaggacga-3′ (SEQ ID NO:48) and 5′-cgcacttggtcccgaagcgc-3′ (SEQ ID NO:49); introns 3 and 4: 5′-gcgcttcgggaccaagtgcg-3′ (SEQ ID NO:50) and 5′-cggggaaggagacctcagcgt-3′ (SEQ ID NO:51); Intron 5: 5′-ggacaaggacagcgttcag-3′ (SEQ ID NO:52) and 5′-ctcccgtagaggccattg-3′ (SEQ ID NO:53).

FIG. 22B is a diagram depicting the location of CpG dinucleotide sequences (vertical lines) within the LHX3 locus.

FIG. 22C is a diagram depicting the structure of LHX3 protein isoforms and correlation of protein domains with the gene exon structure.

FIG. 23A is an image depicting the chromosomal localization of the human LHX3 gene by fluorescence in situ hybridization (FISH). The image depicts a metaphase chromosome spread demonstrating the presence of the LHX3 gene (large arrow) on both Chr 9s identified by the centromeric Chr 9 probe (CEP 9; VYSIS Inc.) indicated by the small arrow at the terminal region of band 9q34.3.

FIG. 23B is an image of an ideogram of Chromosome 9 demonstrating the location of LHX3.

FIG. 23C is an image depicting the co-localization of LHX3 gene probe (large arrow) with the chromosome subtelomeric probe (TelVysion 9q; VYSIS Inc.). The centromere is marked using a green fluorescent probe (CEP 9; VYSIS Inc.) indicated by the small arrow.

FIG. 24 is an image depicting the amino acid sequence of human Lhx3a (SEQ ID NO:10). The amino acids of LIM Domain 1 (amino acids 31-81) and LIM Domain 2 (amino acids 90-144) are underlined. Further, the amino acids of homeodomain (amino acids 156-220) are underlined. The protein is about 397 amino acids in length.

FIG. 25 is an image depicting the amino acid sequence of human Lhx3b (SEQ ID NO:12). The amino acids of LIM Domain 1 (amino acids 36-86) and LIM Domain 2 (amino acids 95-149) are underlined. Further, the amino acids of homeodomain (amino acids 161-225) are underlined. The protein is about 402 amino acids in length.

FIG. 26, comprising FIGS. 26A-26J, is an image depicting the nucleic acid sequence of the genomic DNA encoding human Lhx3 (SEQ ID NO:22). Nucleotides representing introns are capitalized.

FIG. 27, comprising FIGS. 27A, 27B, and 27C, is an image depicting the nucleic acid sequence of porcine Lhx3a (SEQ ID NO:13).

FIG. 28 is an image depicting the amino acid sequence of porcine Lhx3a (SEQ ID NO:14).

FIG. 29, comprising FIGS. 29A and 29B, is an image depicting the nucleic acid sequence of porcine Lhx3b (SEQ ID NO:15).

FIG. 30 is an image depicting the amino acid sequence of porcine Lhx3b (SEQ ID NO:16).

### DETAILED DESCRIPTION OF THE INVENTION

The present invention provides the nucleic acid and amino acid sequences of the human and porcine Lhx3/P-LIM/LIM-3, which sequences are essential for use of the gene in combating pituitary disease. The present invention further provides the sequences of various isoforms of human Lhx3 which differ in their ability to act as transcription factors.

It has been discovered, as disclosed herein, that at least two protein isoforms of the human Lhx3 gene (hLhx3) are expressed in humans, i.e., hLhx3a and hLhx3b, and that these isoforms differ in their DNA binding abilities and in their ability to trans-activate pituitary gene targets (e.g., α-glycoprotein subunit promoter [α-GSU] and thyroid-stimulating hormone β promoter [TSHβ]). The data disclosed herein demonstrate that hLhx3 is the first LIM homeodomain class transcriptional regulator to be identified having isoforms possessing different functional properties, i.e., hLhx3a and hLhx3b.

The invention relates to isolated nucleic acids encoding mammalian Lhx3 including various protein isoforms thereof (e.g., hLhx3a, hLhx3b, and the like).

Further, the invention relates to various recombinant nucleic acid constructs comprising a nucleic acid encoding at least a portion of a mammalian Lhx3 covalently linked to another nucleic acid encoding a tag polypeptide. The tag portion of the polypeptide allows, among other things, the detection and localization of expression of Lhx3 in a cell as well as the purification of the protein from and quantitation of expression in a cell.

The invention also relates to recombinant nucleic acids comprising a promoter/regulatory sequence that interacts with Lhx3 such that a reporter nucleic acid operably linked to the promoter/regulatory sequence is activated.

Further, the present invention relates to isolated proteins encoded by the afore-mentioned nucleic acids and uses for the various nucleic acids and proteins.

The invention also provides diagnostic assays for identifying the presence or absence of a mutation characterized by a mutation of a gene encoding a polypeptide of the invention. The assays also identify altered levels of expression of an Lhx3 polypeptide of the invention, and identify altered levels of activity of Lhx3, wherein such altered levels are potentially associated with and/or mediate a disease, disorder or condition of the pituitary.

In another aspect, the invention provides a method for identifying a compound that binds with or modulates the activity of a polypeptide of the invention. In general, such methods entail measuring a biological activity of the polypeptide in the presence and absence of a test compound and identifying those compounds which bind with or alter the activity of the polypeptide. Such activity-modulating compounds are important potential therapeutics for treatment of diseases, disorders or conditions of the pituitary. This is because altered Lhx3 activity likely mediates such disease, disorder or condition such that a compound that modulates the activity can be used to treat the pituitary disease, disorder or condition.

The invention also features methods for identifying a compound which modulates the expression of a polypeptide or nucleic acid of the invention by measuring the expression of the polypeptide or nucleic acid in the presence and absence of the compound. This compound is a potential therapeutic for a disease, disorder or condition of the pituitary mediated by altered expression of Lhx3. That is, where higher or lower level of expression of a peptide or nucleic acid of the expression mediates a disease, disorder or condition of the pituitary, a compound that modulates that altered expression is useful for treating the disease, disorder or condition.

Moreover, the present invention relates to genomic mapping of the human Lhx3 gene. This is important because this gene had not, until the present invention, been identified or characterized in humans. An understanding of the function of the human Lhx3 gene is crucial to the elucidation of the molecular basis of certain human pituitary diseases, disorders, or conditions, and for the development of diagnostics and therapeutics for such diseases, disorders, or conditions.

Definitions

As used herein, each of the following terms has following meaning.

The articles “a” and “an” are used herein to refer to one or to more than one (i.e., to at least one) of the grammatical object of the article. By way of example, “an element” means one element or more than one element.

As used herein, the term “adjacent” is used to refer to nucleotide sequences which are directly attached to one another, having no intervening nucleotides. By way of example, the pentanucleotide<5′-AAAAA-3′ is adjacent the trinucleotide 5′-TTT-3′ when the two are connected thus: 5′-AAAAATTT-3′ or 5′-TTTAAAAA-3′, but not when the two are connected thus: 5′-AAAAACTTT-3′.

As used herein, the phrase “allelic variant” refers to a nucleotide sequence which occurs at a given locus or to a polypeptide encoded by the nucleotide sequence.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, to “alleviate” a disease, disorder or condition mediated by altered expression of hLhx3 means reducing the severity of the symptoms of the disease, disorder, or condition. This includes, but is not limited to, affecting the level of binding of hLhx3 with a nucleic acid that specifically binds with hLhx3, or affecting the level of expression of hLhx3 in a patient compared with these same parameters in the patient prior to or in the absence of the method of treatment.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator” as the term is used herein, is meant any device including, but not limited to, a hypodermic syringe, a pipette, a nebulizer, and the like, for administering the a nucleic acid, protein, and/or composition of the invention to a mammal.

By “biological activity” or “activity” as the term is used herein, is meant that the protein has the ability to interact with its associated protein(s) (e.g., synergists such as Pit-1) and effectuate its normal function(s) within the cell. More specifically, the biological activity of Lhx3 encompasses, but is not limited to, binding a nucleic acid that specifically binds with Lhx3 (e.g., the αGSU promoter sequence, the Lhx3 consensus binding sequence, and the like), the ability to act in synergy with pituitary transcription factors (e.g., Pit-1) to induce promoters such as TSHβ promoters, the ability to activate the gene encoding Pit-1, the ability to activate the gene encoding prolactin, and the ability to induce a pituitary hormone gene promoter (e.g., αGSU promoter).

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g. amino acid residues in a protein export signal sequence).

By “complementary to a portion or all of the nucleic acid encoding Lhx3” is meant a sequence of nucleic acid which does not encode Lhx3 protein. Rather, the sequence which is identical to the non-coding strand of the nucleic acid encoding Lhx3 and thus, does not encode Lhx3 protein.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand. “Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

“Homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGCG5′ share 50% homology.

A first oligonucleotide anneals with a second oligonucleotide with “high stringency” if the two oligonucleotides anneal under conditions whereby only oligonucleotides which are at least about 60%, more preferably at least about 65%, even more preferably at least about 70%, yet more preferably at least about 80%, and preferably at least about 90% or, more preferably, at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 1989, Molecular Cloning: A Laboratory Manual, Cold Spring Harbor Laboratory, New York).

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example, at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty 3; match reward 1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the human proteins described herein are within the scope of the invention. Nucleic acid molecules corresponding to natural allelic variants and homologs of a cDNA of the invention can be isolated based on their identity to human nucleic acid molecules using the human cDNAs, or a portion thereof, as a hybridization probe according to standard hybridization techniques under stringent hybridization conditions. For example, a cDNA encoding a soluble form of a membrane-bound protein of the invention can be isolated based on its hybridization with a nucleic acid molecule encoding all or part of the membrane-bound form. Likewise, a cDNA encoding a membrane-bound form can be isolated based on its hybridization with a nucleic acid molecule encoding all or part of the soluble form.

A “coding region” of a gene consists of-the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

A first region of an oligonucleotide “flanks” a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 20 nucleotides in length, typically, at least about 100 nucleotides, more typically, from about 100 to about 500 nucleotides, typically at least about forty contiguous amino acids, preferably at least about 500 to about 1,000 nucleotides, even more preferably at least about 1,000 nucleotides to about 2,000 nucleotides, yet even more preferably at least about 2,000 to about 3,500, and most preferably, the nucleic acid fragment will be greater than about 3,500 nucleotides in length.

As used herein, the term “fragment” as applied to a polypeptide, may ordinarily be at least about seven contiguous amino acids, typically, at least about fifteen contiguous amino acids, more typically, at least about thirty contiguous amino acids, typically at least about forty contiguous amino acids, preferably at least about fifty amino acids, even more preferably at least about sixty amino acids and most preferably, the peptide fragment will be greater than about sixty contiguous amino acids in length.

As applied to a protein, a “fragment” of Lhx3 is about 10 amino acids in length. More preferably, the fragment of a Lhx3 is at least about 20 amino acids, even more preferably at least about 30 amino acids, more preferably at least about 100 amino acids, even more preferably, at least about 70, yet more preferably, at least about 100, even more preferably, at least about 200, yet more preferably, at least about 300, more preferably, at least about 400 amino acids in length, and even more preferably, at least about 450 amino acids in length.

As used herein, the term “hybridizes under stringent conditions” is intended to describe conditions for hybridization and washing under which nucleotide sequences at least 60% (65%, 70%, preferably 75%) identical to each other typically remain hybridized with each other. Such stringent conditions are known to those skilled in the art and can be found in In: Current Protocols in Molecular Biology, at 6.3.1-6.3.6, John Wiley & Sons, N.Y. (1989). An example of stringent hybridization conditions are hybridization in 6x sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to the sequence of any of SEQ ID NOs: 7, 9, and 11, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

A “genomic DNA” is a DNA strand which has a nucleotide sequence homologous with a gene. By way of example, both a fragment of a chromosome and a cDNA derived by reverse transcription of a mammalian mRNA are genomic DNAs.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

In addition, when the term “homology” is used herein to refer to the nucleic acids and proteins, it should be construed to be applied to homology at both the nucleic acid and the amino acid levels.

By the term “human Lhx3” (“hLhx3”), as used herein, is meant any nucleic acid encoding an Lhx3 protein wherein the nucleic acid has at least about 89% homology to hLhx3 (SEQ ID NO:7). The term encompasses human Lhx3 isoforms including, but not limited to, hLhx3a, hLhx3b, and the like.

By the term “human Lhx3” (“hLhx3”), as used herein, is meant any protein comprising an Lhx3 protein having at least about 94.5% homology to hLhx3 (SEQ ID NO:8). The term encompasses human Lhx3 isoforms including, but not limited to, hLhx3a, hLhx3b, and the like.

By the term “porcine Lhx3” (“hLhx3”), as used herein, is meant any nucleic acid encoding an Lhx3 protein wherein the nucleic acid has at least about 88% homology to pLhx3 (SEQ ID NO:1). The term encompasses porcine Lhx3 isoforms including, but not limited to, pLhx3a, pLhx3b, and the like.

By the term “porcine Lhx3” (“pLhx3”), as used herein, is meant any protein comprising an Lhx3 protein having at least about 93% homology to pLhx3 (SEQ ID NO:2). The term encompasses porcine Lhx3 isoforms including, but not limited to, pLhx3a, pLhx3b, and the like.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

“Mutants,” “derivatives,” and “variants” of the peptides of the invention (or of the DNA encoding the same) are peptides which may be altered in one or more amino acids (or in one or more base pairs) such that the peptide (or DNA) is not identical to the sequences recited herein, but has the same property as the peptides disclosed herein, in that the peptide has the property of binding with αGSU promoter sequence PGBE, trans-activating certain pituitary trophic hormone gene promoters, synergizing with pituitary transcription factors such as Pit-1 to induce transcriptions of, e.g., TSHβ promoter sequence.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

Preferably, when the nucleic acid encoding the desired protein further comprises a promoter/regulatory sequence, the promoter/regulatory is positioned at the 5′ end of the desired protein coding sequence such that it drives expression of the desired protein in a cell.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “expression of a nucleic acid” as used herein means the synthesis of the protein product encoded by the nucleic acid.

The use of the term “DNA encoding” should be construed to include the DNA sequence which encodes the desired protein and any necessary 5′ or 3′ untranslated regions accompanying the actual coding sequence.

By the term “positioned at the 5′ end” as used herein, is meant that the promoter/regulatory sequence is covalently bound to the 5′ end of the nucleic acid whose expression it regulates, at a position sufficiently close to the 5′ start site of transcription of the nucleic acid so as to drive expression thereof.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

A “polyadenylation sequence” is a polynucleotide sequence which directs the addition of a poly A tail onto a transcribed messenger RNA sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced. i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574). Similarly, as exemplified herein, luciferase can also be used as a reporter gene to detect expression of a protein of interest (i.e., Lhx3) as can green fluorescent protein (GFP).

A “restriction site” is a portion of a double-stranded nucleic acid which is recognized by a restriction endonuclease.

A portion of a double-stranded nucleic acid is “recognized” by a restriction endonuclease if the endonuclease is capable of cleaving both strands of the nucleic acid at the portion when the nucleic acid and the endonuclease are contacted.

By the term “specifically binds,” as used herein, is meant a compound, e.g., a protein, a nucleic acid, an antibody, and the like, which recognizes and binds a specific molecule, but does not substantially recognize or bind other molecules in a sample.

A first oligonucleotide anneals with a second oligonucleotide “with high stringency” if the two oligonucleotides anneal under conditions whereby only oligonucleotides which are at least about 60%, preferably at least about 65%, more preferably at least about 70%, even more preferably at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 1989, Molecular Cloning: A Laboratory Manual, Cold Spring Harbor Laboratory, New York).

As used herein, the term “substantially pure” describes a compound, e.g., a nucleic acid, protein or polypeptide, which has been separated from components which naturally accompany it. Typically, a compound is substantially pure when at least about 10%, preferably at least about 20%, more preferably at least about 50%, still more preferably at least about 75%, even more preferably at least about 90%, and most preferably at least about 99% of the total material (by volume, by wet or dry weight, or by mole percent or mole fraction) in a sample is the compound of interest. Purity can be measured by any appropriate method, e.g., by column chromatography, gel electrophoresis or HPLC analysis.

A compound, e.g., a nucleic acid, a protein or polypeptide is also “substantially purified” when it is essentially free of naturally associated components or when it is separated from the native contaminants which accompany it in its natural state. Thus, a “substantially pure” preparation of a nucleic acid, as used herein, refers to a nucleic acid sequence which has been purified from the sequences which flank it in a naturally occurring state, e.g., a DNA fragment which has been removed from the sequences which are normally adjacent to the fragment in a genome in which it naturally occurs.

Similarly, a “substantially pure” preparation of a protein or a polypeptide, as used herein, refers to a protein or polypeptide which has been purified from components with which it is normally associated in its naturally occurring state. A substantially pure peptide can be purified by following known procedures for protein purification, wherein an immunological, enzymatic or other assay is used to monitor purification at each stage in the procedure. Protein purification methods are well known in the art, and are described, for example in Deutscher et al. (1990, In: Guide to Protein Purification, Harcourt Brace Jovanovich, San Diego).

A factor “synergizes” with Lhx3 when the combined activities of the factor and Lhx3 is greater than the sum of the individual activities of the factor and of Lhx3 separately.

By the term “exogenous nucleic acid” is meant that the nucleic acid has been introduced into a cell or an animal using technology which has been developed for the purpose of facilitating the introduction of a nucleic acid into a cell or an animal.

A cell that comprises an exogenous nucleic acid is referred to as a “recombinant cell.” Such a cell may be a eukaryotic cell or a prokaryotic cell. A gene which is expressed in a recombinant cell wherein the gene comprises a recombinant polynucleotide, produces a “recombinant polypeptide.”

By “tag” polypeptide is meant any protein which, when linked by a peptide bond to a protein of interest, may be used to localize the protein, to purify it from a cell extract, to immobilize it for use in binding assays, or to otherwise study its biological properties and/or function. A chimeric (i.e., fusion) protein containing a “tag” epitope can be immobilized on a resin which binds the tag. Such tag epitopes and resins which specifically bind them are well known in the art and include, for example, tag epitopes comprising a plurality of sequential histidine residues (His6), which allows isolation of a chimeric protein comprising such an epitope on nickel-nitrilotriacetic acid-agarose, a hemagglutinin (HA) tag epitope allowing a chimeric protein comprising such an epitope to bind with an anti-HA-monoclonal antibody affinity matrix, a myc tag epitope allowing a chimeric protein comprising such an epitope to bind with an anti-myc-monoclonal antibody affinity matrix, a glutathione-S-transferase tag epitope, and a maltose binding protein (MBP) tag epitope, which can induce binding between a protein comprising such an epitope and a glutathione- or maltose-Sepharose column, respectively. Production of proteins comprising such tag epitopes is well known in the art and is described in standard treatises such as Sambrook et al., 1989, and Ausubel et al., supra. Likewise, antibodies to the tag epitope (e.g., anti-HA, anti-myc antibody 9E 10, and the like) allow detection and localization of the fusion protein in, for example, Western blots, ELISA assays, and immunostaining of cells.

By the term “trans-activate” as used herein, is meant activation of a gene by the interaction of a protein (i.e., acting in trans), with the regulatory elements operably linked to the nucleic acid encoding the gene (i.e., the cis elements of the gene).

As used herein, to “treat” means reducing the frequency with which symptoms are experienced by a patient.

By the term “vector” as used herein, is meant any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of the nucleic acid encoding Lhx3 to a cell patient, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

Description

I. Isolated Nucleic Acids

A. Sense Nucleic Acids

The present invention provides a novel isolated nucleic acid (SEQ ID NO:7) encoding the human Lhx3 protein (hLhx3 [SEQ ID NO:8]). In addition, the invention provides an isolated nucleic acid (SEQ ID NO:1) encoding the porcine Lhx3 protein (pLhx3 [SEQ ID NO:2]).

The invention includes an isolated nucleic acid encoding a mammalian Lhx3, which isolated nucleic acid is, preferably, greater that about 88% homologous to pLhx3 (SEQ ID NO:1). More preferably, the isolated nucleic acid encoding a mammalian Lhx3 is at least about 90% homologous, more preferably, at least about 95% and even more preferably, at least about 99% homologous to pLhx3 (SEQ ID NO:1). More preferably, the isolated nucleic acid encoding a mammalian Lhx3 is pLhx3. Most preferably, the sequence of an isolated nucleic acid encoding a mammalian Lhx3 is SEQ ID NO:1.

The invention includes an isolated nucleic acid encoding a mammalian Lhx3, which isolated nucleic acid is, preferably, at least about 87.5% homologous to hLhx3 (SEQ ID NO:9). More preferably, the isolated nucleic acid encoding a mammalian Lhx3 is at least about 90% homologous, even more preferably, at least about 95% homologous, more preferably, at least about 99% homologous to hLhx3 (SEQ ID NO:9). More preferably, the isolated nucleic acid encoding a mammalian Lhx3 is hLhx3a. Most preferably, the sequence of an isolated nucleic acid encoding a mammalian Lhx3 is SEQ ID NO:9.

The invention also includes a nucleic acid encoding a mammalian hLhx3b protein. Preferably, the nucleic acid encoding a mammalian Lhx3b is at least about 88% homologous to human Lhx3b (SEQ ID NO:11). More preferably, the isolated nucleic acid encoding a mammalian Lhx3b is at least about 90% homologous, more preferably, at least about 95% and even more preferably, at least about 99% homologous to (SEQ ID NO:11). More preferably, the isolated nucleic acid encoding a mammalian Lhx3b is human Lhx3b. Most preferably, the isolated nucleic acid encoding a mammalian hLhx3b is SEQ ID NO:11.

In addition to the nucleotide sequences of any of SEQ ID NOs: 7, 9, and 11, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus.

Accordingly, in another embodiment, an isolated hLhx3 nucleic acid molecule of the invention is at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, 5000, 5500, 6000, 6500, 7000, 7500, 8000, 8500, or 9000) nucleotides in length and hybridizes under stringent conditions to the nucleic acid molecule comprising the nucleotide sequence, preferably the coding sequence, of any of SEQ ID NOs: 7, 9, and 11, or a complement thereof.

The isolated nucleic acid of the invention should be construed to include an RNA or a DNA sequence encoding an Lhx3 protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

One skilled in the art would appreciate, based upon the disclosure provided in the specification, that the invention includes a fragment of the nucleic acids of the invention. As used herein, the term “fragment” as applied to a nucleic acid, may ordinarily be at least about 20 nucleotides in length, typically, at least about 50 nucleotides, more typically, from about 100 to about 400 nucleotides, typically at least about forty contiguous amino acids, preferably at least about 400 to about 800 nucleotides, even more preferably at least about 800 nucleotides to about 1200 nucleotides, more preferably, at least about 1200 nucleotides to about 1500 nucleotides, even more preferably, at least about 1500 nucleotides to about 2000 nucleotides, yet even more preferably, at least about 2000 nucleotides to about 2500 nucleotides, more preferably, at least about 2500 nucleotides to about 3000 nucleotides, even more preferably, at least about 3000 nucleotides to about 3500 nucleotides, yet more preferably, at least about 3500 nucleotides to about 4000 nucleotides, more preferably, at least about 4000 nucleotides to about 4500 nucleotides, and more preferably, the nucleic acid fragment will be greater than about 4500 nucleotides in length.

One skilled in the art would understand, based upon the disclosure provided herein, that there is a portion or portions of each Lhx3 isoform that is highly homologous with the same portion of another isoform (i.e., LIM domain1, LIM domain 2, homeodomain, and the like. Further, the skilled artisan would understand, based upon the disclosure provided herein, that there are fragments or portions of Lhx3 that are not as highly conserved (e.g., the 5′ region of a nucleic acid encoding Lhx3 from about nucleotide −104 to about nucleotide 78 (approximately 182 bp) of an isolated nucleic acid encoding hLhx3a (SEQ ID NO:9). Preferably, the fragment has the sequence

| | |
|---|---|
| 5′-ggcacgagccccgcacgacgcggcgggacttgggagccccgaaccctccaggggacgctgacctcggaggagcgcgt | (SEQ ID NO:23) |
| ctcgcgccactcggcctggtggccgcgATGCTGCTGGAAACGGGGCTCGAGCGCGACCGA | |
| GCGAGGCCCGGGGCCGCCGCCGTCTGCACCTTGGGCGGGACTCGG-3′. | |

Therefore, the present invention encompasses fragments encompassing conserved and non-conserved portions of Lhx3.

The present invention includes an isolated nucleic encoding a portion of a mammalian Lhx3 protein. Preferably, the nucleic acid encoding a mammalian Lhx3 is at least about 45% homologous to the unique portion of human Lhx3a (SEQ ID NO:23). The nucleic acid (SEQ ID NO:23) comprises from about nucleotide −104 to about nucleotide 78 of SEQ ID NO:9. More preferably, the isolated nucleic acid encoding a mammalian Lhx3a is at least about 50% homologous, more preferably, at least about 60%, homologous, preferably, at least about 65%, more preferably, at least about 70%, even more preferably, at least about 75%, more preferably, at least about 80%, even more preferably, at least about 85%, yet more preferably, at least about 90% homologous, more preferably, at least about 95% and even more preferably, at least about 99% homologous to (SEQ ID NO:23). More preferably, the isolated nucleic acid encoding a mammalian Lhx3a is human Lhx3a. Most preferably, the isolated nucleic acid encoding a mammalian Lhx3 is SEQ ID NO:23.

The present invention includes an isolated nucleic encoding a portion of a mammalian Lhx3 protein. Preferably, the nucleic acid encoding a mammalian Lhx3 is at least about 67% homologous to human Lhx3b (SEQ ID NO:24). The nucleic acid (SEQ ID NO:24) comprises from about nucleotide −119 to about nucleotide 93 of SEQ ID NO:11. More preferably, the isolated nucleic acid encoding a mammalian Lhx3b is at least about 70%, preferably, at least about 75%, more preferably, at least about 80%, even more preferably, at least about 85%, more preferably, at least about 90%, even more preferably, at least about 95%, and even more preferably, at least about 99% homologous to (SEQ ID NO:24). More preferably, the isolated nucleic acid encoding a mammalian Lhx3b is human Lhx3b. Most preferably, the isolated nucleic acid encoding a mammalian Lhx3 is

SEQ ID NO:24 (i.e., 5′-
cgcagcgcccagcagcacccggagtcgcttggacgccggttcggggctattgcggggtggcgtcgctgggcccgggaaa
gttcgggactggagagtggcgacgccgggcggcgggacccATGGAGGCGCGCGGGGAGCTGGGC
CCGGCCCGGGAGTCGGCGGGAGGCGACCTGCTGCTAGCACTGCTGGCGCG
GAGGGCAGACCTGCGCCGA-3′).

The present invention should not be construed as being limited solely to the nucleic and amino acid sequences disclosed herein. Once armed with the present invention, it would be readily apparent to one skilled in the art that other nucleic acids encoding porcine and human Lhx3 proteins, and isoforms thereof, may be obtained by following the procedures described herein in the experimental details section for the isolation of the human and porcine nucleic acids encoding Lhx3 proteins, as well as those procedures used to identify and isolate human Lhx3 and porcine Lhx3, and the respective polypeptides encoded thereby, as disclosed elsewhere herein.

Further, the invention should be construed to include naturally occurring variants or recombinantly derived mutants of wild type Lhx3 DNA sequences, which variants or mutants render the protein encoded thereby either more, less, or just as biologically active as the full-length wild type Lhx3 of the invention. Any number of procedures can be used for the generation of mutant, derivative or variant forms of Lhx3, using recombinant DNA methodology well known in the art such as, for example, that described in Sambrook et al. (1989, Molecular Cloning: A Laboratory Manual, Cold Spring Harbor Laboratory Press, New York), Ausubel et al. (1997, Current Protocols in Molecular Biology, Green & Wiley, New York), and elsewhere herein.

As will be apparent from the experiments described herein, Lhx3 comprises a binding domain which mediates Lhx3 binding to, inter alia, the αGSU −350 to −323 bp Lhx3 binding site (i.e., the pituitary glycoprotein basal element, PGBE) and to the Lhx3 consensus binding site.

The invention also includes a nucleic acid encoding Lhx3, and mutants, derivatives, variants, and fragments thereof, that can retain biological activity. Such variants, ie., analogs of Lhx3, include proteins or polypeptides which have been or may be modified using recombinant DNA technology such that the protein or polypeptide possesses additional properties which enhance its suitability for use in the methods described herein, for example, but not limited to, variants conferring enhanced stability on the Lhx3 protein, enhanced specific binding of Lhx3 with, for example,

PGBE, enhanced binding of the homeodomain of Lhx3 with DNA, and the like.

Procedures for the introduction of amino acid changes in a protein or polypeptide by altering the DNA sequence encoding the polypeptide are well known in the art and are also described in Sambrook et al. (1989, supra); Ausubel et al. (1997, supra).

The invention includes a nucleic acid encoding a mammalian Lhx (e.g., hLhx3, pLhx3) wherein a nucleic acid encoding a tag polypeptide is covalently linked thereto. That is, the invention encompasses a chimeric nucleic acid wherein a nucleic acid sequence encoding a tag polypeptide is covalently linked to a nucleic acid encoding at least one of porcine Lhx3 and human Lhx3. Such chimeric (i.e., fusion) tag polypeptides are well known in the art and include, for instance, myc, myc-pyruvate kinase (myc-PK), His₆, maltose biding protein (MBP), glutathione-S-transferase (GST), and green fluorescence protein (GFP). However, the invention is not limited to the nucleic acids encoding the above-listed tag polypeptides. Rather, any nucleic acid sequence encoding a polypeptide which may function in a manner substantially similar to these tag polypeptides should be construed to be included in the present invention.

A nucleic acid encoding a protein of interest (e.g., pLhx3, hLhx3, and any mutant, derivative, variant, or fragment thereof) comprising a nucleic acid encoding a tag polypeptide and a fusion protein produced therefrom can be used to, among other things, localize Lhx3 within a cell and to study expression, localization, and role(s) of the tagged protein in a cell before, during, and/or after exposing the cell to a test compound. Further, addition of a tag to a protein of interest facilitates isolation and purification of the “tagged” protein such that the protein of interest can be easily produced and purified.

In other related aspects, the invention includes a nucleic acid encoding a mammalian Lhx3 operably linked to a nucleic acid comprising a promoter/regulatory sequence such that the nucleic acid is preferably capable of directing expression of the protein encoded by the nucleic acid.

Expression of Lhx3 either alone or fused to a detectable tag polypeptide in cells which either do not normally express Lhx3 or which do not express Lhx3 comprising a tag polypeptide, can be accomplished by operably linking the nucleic acid encoding Lhx3 to a promoter/regulatory sequence which serves to drive expression of the protein, with or without a tag polypeptide, in cells in which the exogenous nucleic acid (i.e. transgene) is introduced.

Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like. Moreover, inducible and tissue specific expression of the nucleic acid encoding Lhx3 may be accomplished by placing the nucleic acid encoding Lhx3, with or without a tag polypeptide, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein encoded by a nucleic acid operably linked to the promoter/regulatory sequence.

Expressing Lhx3 using a promoter/regulatory sequence allows the isolation of large amounts of recombinantly produced protein. Further, where the lack or decreased level of Lhx3 expression causes a disease, disorder, or condition associated with such expression, the expression of the protein driven by a promoter/regulatory sequence can provide useful therapeutics including, but not limited to, gene therapy whereby the protein is provided.

B. Antisense Nucleic Acids

In certain situations, it may be desirable to inhibit expression of mammalian Lhx3 in a cell that would otherwise express the protein. For example, the invention encompasses, but is not limited to, inhibiting Lhx3 expression where over-expression (e.g., expression at a level higher than the normal level) of Lhx3 mediates a disease, disorder or condition of the pituitary. Further, inhibiting expression of mammalian Lhx3 in a cell provides an in vitro model system for the study of a disease, disorder or condition of the pituitary mediated by an inhibition of Lhx3 expression. Such a cell can provide a useful in vitro model system for the study of, for instance, the mechanism of such disease, disorder or condition as well as for the identification of therapeutic treatment thereof.

Therefore, the invention includes compositions useful for inhibition of expression of Lhx3, and isoforms thereof. One such method of the invention features an isolated nucleic acid complementary to a portion or all of a nucleic acid encoding an Lhx3 which is in an antisense orientation with respect to transcription. Preferably, the isolated antisense nucleic acid useful for inhibition of Lhx3 expression shares at least 88% homology with a sequence complementary to at least one of SEQ ID NO: l, SEQ ID NO:7, SEQ ID NO:9, SEQ ID NO:11, SEQ ID NO:13, and SEQ ID NO:15, or a portion thereof.

More preferably, the isolated antisense nucleic acid complementary to an isolated nucleic encoding a mammalian Lhx3 is at least about 89%, preferably at least about 90%, more preferably, at least about 95% and even more preferably, at least about 99% homologous to a sequence complementary to at least one of SEQ ID NO:1, SEQ ID NO:7, SEQ ID NO:9, SEQ ID NO:11, SEQ ID NO:13, and SEQ ID NO:15. More preferably, the isolated antisense nucleic acid complementary to an isolated nucleic acid encoding a mammalian Lhx3 is an isolated antisense nucleic acid complementary to an isolated nucleic acid encoding at least one of a porcine Lhx3, a human Lhx3, a hLhx3a, a hLhx3b, a porcine Lhx3a and a porcine Lhx3b. Most preferably, the isolated antisense nucleic acid complementary to an isolated nucleic acid encoding a mammalian hLhx3 is an isolated nucleic acid complementary to at least one of SEQ ID NO:1, SEQ ID NO:7, SEQ ID NO:9, and SEQ ID NO:11.

The above-referred to antisense nucleic acids serve to inhibit the expression, function, or both, of human Lhx3 and porcine Lhx3.

Additionally, the use of ribozymes to effect an inhibition of expression of Lhx3 is contemplated in the present invention, as is the use of any other means which would effect a reduction in expression of Lhx3. Means of inhibiting expression of a desired nucleic acid encoding a protein of interest are well-known in the art and are described, for example, in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York).

One skilled in the art will appreciate, based on the disclosure provided herein, that the level of Lhx3 protein in a cell can be decreased by reducing or inhibiting expression of the Lhx3 gene. Thus, the level of Lhx3 protein in a cell can be decreased using a molecule or compound that inhibits or reduces gene expression such as, for example, antisense molecules, ribozymes, or double-stranded RNA as described in, for example, Wianny and Kernicka-Goetz (2000, Nature Cell Biol. 2:70-75).

In a one embodiment, the Lhx3 modulating sequence is a Lhx3 antisense nucleic acid sequence which is encoded by a plasmid vector and which is used to transfect a mammalian cell thereby causing reduced endogenous expression of Lhx3 protein in the cells. However, as stated previously herein, the invention should not be construed to be limited to inhibiting expression of Lhx3 by transfection of cells with antisense molecules. Rather, the invention encompasses other methods known in the art for inhibiting expression of Lhx3 protein in cells including, but not limited to, the use of ribozymes, double stranded RNA, and expression of a non-functional Lhx3 under the control of, for example, an inducible promoter, and the like.

With regard to using antisense molecules to inhibit gene expression, such use is well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243, incorporated by reference herein in its entirety).

Ribozymes and their use for inhibiting gene expression are also well known in the art (see, e.g., Cech et al., 1992, J. Biol. Chem. 267:17479-17482; Hampel et al., 1989, Biochemistry 28:49294933; Eckstein et al., International Publication No. WO 92107065; Altman et al., U.S. Pat. No. 5,168,053, incorporated by reference herein in its entirety). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is that, because they are sequence-specific, only mRNAs with particular sequences are inactivated.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of Lhx3 may be designed by incorporating target sequences into the basic ribozyme structure which are complementary to the mRNA sequence of the Lhx3 encoded by Lhx3 or having at least about 94% homology to mammalian Lhx3 (SEQ ID NOs:7, 9, 11, 13, and 15). Ribozymes targeting Lhx3 can be synthesized using commercially available reagents (Applied Biosystems, Inc., Foster City, Calif.) or they may be genetically expressed from DNA encoding them.

II Vectors

The invention also includes a vector comprising a nucleic acid encoding a mammalian Lhx3. Methods for incorporating a desired nucleic acid into a vector and the choice of vectors is well-known in the art as described in, for example, Sambrook et al., supra, and Ausubel et al., supra.

Further, the invention encompasses expression vectors and methods for the introduction of exogenous nucleic acid encoding Lhx3 into a cell with concomitant expression of the exogenous nucleic acid in the cell using such methods as those described in, for example, Sambrook et al. (1989, supra), and Ausubel et al. (1997, supra), and as disclosed elsewhere herein.

Selection of any particular plasmid vector or other DNA vector is not a limiting factor in this invention and a wide plethora vectors are well-known in the art (see, e.g., Sambrook et al., supra, and Ausubel et al., supra.). Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook, supra, and Ausubel, supra.

The invention includes also cells, viruses, proviruses, and the like containing such vectors. Methods for producing cells comprising vectors and/or exogenous nucleic acids are well-known in the art. See, e.g., Sambrook et al., supra; Ausubel et al., supra.

The nucleic acids encoding Lhx3 can be cloned into various plasmid vectors. However, the present invention should not be construed to be limited to plasmids or to any particular vector. Instead, the present invention should be construed to encompass a wide plethora of vectors which are readily available and/or well-known in the art.

III. Recombinant Cells

Additionally, the nucleic and amino acids of the invention can be used to produce recombinant cells which are useful tools for the study of Lhx3, the identification of novel Lhx3-based therapeutics, and for elucidating the cellular role(s) of Lhx3 and its isoforms, among other things.

Further, the nucleic and amino acids of the invention can be used diagnostically, either by assessing the level of gene expression or protein expression and the biological activity of the protein, to assess severity and prognosis of a disease, disorder, or condition associated with altered level of Lhx3 gene expression.

The invention also includes expression of human Lhx3 in a cell where it is not normally expressed or expression of Lhx3-tagged fusion protein in cells where this fusion protein is not normally expressed. In a preferred embodiment, human Lhx3 nucleic acid can be tagged with a nucleic acid expressing a tag polypeptide to transfect a mammalian cell. Plasmid constructs containing Lhx3, or mutants, variants, derivatives and fragments thereof, can be cloned into a wide variety of vectors including a vector comprising a nucleic acid encoding a tag polypeptide. The plasmids can be introduced into a cell using standard methods well-known in the art (e.g., calcium phosphate, electroporation, and the like). Methods for cloning and introducing an isolated nucleic acid of interest into a cell are exemplified herein and are described in, for example, Sambrook et al. (1989, Molecular Cloning: A Laboratory Manual, Cold Spring Harbor Laboratory Press, New York), Ausubel et al. (1997, Current Protocols in Molecular Biology, Green & Wiley, New York), and other standard treatises.

The present invention also encompasses expression of an isolated Lhx3 of the invention in amphibian and other non-mammalian cells (e.g. yeast, insect, and avian cells) using methods well-known in the art such as those disclosed elsewhere herein. Thus, it is clear that the invention is not limited to any particular vector or to any particular method of introducing the exogenous nucleic acid encoding Lhx3 into a cell.

Expression of proteins of interest (e.g., Lhx3 and isoforms thereof) in a cell, especially when the protein comprises a tag polypeptide, allows localization of the nucleic acid and/or the protein expressed therefrom within the cell under selected conditions such that the function(s) of the protein in the cell can be studied and identified.

One skilled in the art would appreciate, based upon the disclosure provided herein, that the invention also includes expression of Lhx3, and the like, in prokaryotic cells (e.g., bacterial cells such as, for example, E. coli). Accordingly, the invention includes expression of the proteins of the invention in such cells as well.

The invention should not be construed as being limited to these plasmid vectors, bacterial strains, or to these tag polypeptides. Further, the invention is not limited to calcium phosphate transfection or to HeLa cells as exemplified herein. Instead, the invention encompasses other expression vectors and methods for the introduction of exogenous DNA into cells with concomitant expression of the exogenous DNA in the cells such as those described, for example, in Sambrook et al. (1989, supra), and Ausubel et al. (1997, supra).

In one embodiment, the cell line is mammalian cell comprising an expression vector comprising the human Lhx3 cDNA constitutively expressed under the control of a high-level expression promoter/regulatory sequence. The cells may be transfected with constructs which comprise Lhx3 cDNA in either a sense (i.e., sense cells) or an antisense orientation (i.e., antisense cells).

One skilled in the art would further appreciate that selected forms of nucleic acids encoding Lhx3 can be introduced to a cell in order to study the effect of any mutant, derivative, and variant of Lhx3 (e.g., fusion proteins comprising at least a portion of Lhx3 and a tag polypeptide) in this system.

Further, the invention includes a recombinant cell comprising an antisense nucleic acid which cell is a useful model for the study of a disease, disorder, or condition associated with or mediated by inhibition of Lhx3 biosynthesis and for elucidating the role(s) of Lhx3 in such processes. That is, the lack of expression of Lhx3 in patients may indicate, among other things, a disease, disorder or condition. Accordingly, a recombinant (ie., transgenic) cell comprising an antisense nucleic acid complementary to Lhx3 is a useful tool for the study of the mechanism(s) of action of Lhx3 and its role(s) in the cell and for the identification of therapeutics that ameliorate the effects) of decreased levels of Lhx3 expression.

The invention further includes a recombinant cell comprising an isolated nucleic acid encoding Lhx3. The cell can be transiently transfected with a plasmid encoding a portion of the nucleic acid encoding the protein of interest, e.g., Lhx3. The nucleic acid need not be integrated into the cell genome nor does it need to be expressed in the cell. Moreover, the cell may be a prokaryotic or a eukaryotic cell and the invention should not be construed to be limited to any particular cell line or cell type.

When the cell is a eukaryotic cell, the cell may be any eukaryotic cell which, when the isolated nucleic acid of the invention is introduced therein, and the protein encoded by the desired gene is no longer expressed therefrom, a benefit is obtained. Such a benefit may include the fact that there has been provided a system in which lack of expression of the desired gene can be studied in vitro in the laboratory or in a mammal in which the cell resides, a system wherein cells comprising the introduced gene deletion can be used as research, diagnostic and therapeutic tools, and a system wherein animal models are generated which are useful for the development of new diagnostic and therapeutic tools for selected disease, disorder, or condition states in a mammal.

Alternatively, the invention includes a eukaryotic cell which, when the isolated nucleic acid of the invention is introduced therein, and the protein encoded by the desired gene, Le., Lhx3, is expressed therefrom where it was not previously present or expressed in the cell or where it is now expressed at a level or under circumstances different than that before the isolated nucleic acid was introduced, a benefit is obtained. Such a benefit may include the fact that there has been provided a system wherein the expression of the desired gene can be studied in vitro in the laboratory or in a mammal in which the cell resides, a system wherein cells comprising the introduced gene can be used as research, diagnostic and therapeutic tools, and a system wherein animal models are generated which are useful for the development of new diagnostic and therapeutic tools for selected disease states in a mammal (e.g., diseases, disorders or conditions of the pituitary mediated by altered expression or activity of Lhx3).

IV. Isolated polypeptides

The invention also includes an isolated polypeptide encoded by a nucleic acid encoding a mammalian Lhx3 where the amino acid sequence of the polypeptide is preferably, at least about 93% homologous to the amino acid sequence of pLhx3 (SEQ ID NO:2). More preferably, the isolated nucleic acid encodes a mammalian Lhx3 which is at least about 94%, more preferably, at least about 95%, and even more preferably, at least about 99% homologous to porcine Lhx3 (SEQ ID NO:2). More preferably, the isolated nucleic acid encodes a mammalian Lhx3 that is pLhx3. Most preferably, the isolated nucleic acid encodes a mammalian Lhx3 having the amino acid sequence SEQ ID NO:2.

The present invention includes an isolated polypeptide encoded by a nucleic acid encoding a mammalian Lhx3a, or a fragment thereof, wherein the amino acid sequence of the polypeptide encoded by the nucleic acid shares at least about 91% homology with the amino acid sequence of SEQ ID NO:14. Preferably, the nucleic acid encodes a protein that is about 92% homologous, more preferably 93% homologous, even more preferably 95% homologous, and most preferably about 99% homologous to the pLhx3a disclosed herein (SEQ ID NO:14). Even more preferably, the pLhx3a protein encoded by the nucleic acid is SEQ ID NO:14.

In another aspect, the present invention includes an isolated polypeptide encoded by a nucleic acid encoding a mammalian Lhx3b, or a fragment thereof, wherein the polypeptide encoded by the nucleic acid shares at least about 93% homology with the amino acid sequence of SEQ ID NO:16. Preferably, the nucleic acid encodes a protein that is at least about 94% homologous, more preferably at least about 95% homologous, and even more preferably about 99% homologous to the porcine Lhx3b disclosed herein (SEQ ID NO:16). Even more preferably, the pLhx3b protein encoded by the nucleic acid is SEQ ID NO:16.

The invention also includes an isolated polypeptide encoded by a nucleic acid encoding a mammalian Lhx3 where the amino acid sequence of the polypeptide is preferably, at least about 94.5% homologous to the amino acid sequence of hLhx3 (SEQ ID NO:8). More preferably, the isolated nucleic acid encodes a mammalian Lhx3 which is at least about 95%, more preferably, at least about 97%, and even more preferably, at least about 99% homologous to human Lhx3 (SEQ ID NO:8). More preferably, the isolated nucleic acid encodes a mammalian Lhx3 that is hLhx3. Most preferably, the isolated nucleic acid encodes a mammalian Lhx3 having the amino acid sequence SEQ ID NO:8.

In another aspect, the present invention includes an isolated polypeptide encoded by a nucleic acid encoding a mammalian Lhx3a, or a fragment thereof, wherein the amino acid sequence of the polypeptide encoded by the nucleic acid shares at least about 91.5% homology with the amino acid sequence of SEQ ID NO:10. Preferably, the nucleic acid encodes a protein that is about 92% homologous, more preferably about 95% homologous, and most preferably about 99% homologous to the human Lhx3a disclosed herein (SEQ ID NO:10). Even more preferably, the hLhx3a protein encoded by the nucleic acid is SEQ ID NO:10.

In another aspect, the present invention includes an isolated polypeptide encoded by a nucleic acid encoding a mammalian Lhx3b, or a fragment thereof, wherein the amino acid sequence of the polypeptide encoded by the nucleic acid shares at least about 94.5% homology with the amino acid sequence of SEQ ID NO:12. Preferably, the nucleic acid encodes a protein that is about 95% homologous, and most preferably about 99% homologous to the human Lhx3b disclosed herein (SEQ ID NO:12). Even more preferably, the hLhx3b protein encoded by the nucleic acid is SEQ ID NO:12.

The invention also includes an isolated polypeptide comprising a mammalian Lhx3. Preferably, the isolated polypeptide comprising a mammalian Lhx3 is at least about 93% homologous to porcine Lhx3 (SEQ ID NO:2). More preferably, the isolated polypeptide comprising a mammalian Lhx3 is at least about 94%, more preferably, at least about 95%, and more preferably, at least about 99% homologous to porcine Lhx3. More preferably, the isolated polypeptide comprising a mammalian Lhx3 is porcine Lhx3. Most preferably, the isolated polypeptide comprising a mammalian Lhx3 is SEQ ID NO:2.

The invention also includes an isolated polypeptide comprising a mammalian Lhx3. Preferably, the isolated polypeptide comprising a mammalian Lhx3 is at least about 90.5% homologous to SEQ ID NO:14. More preferably, the isolated polypeptide comprising a mammalian Lhx3 is at least about 91%, more preferably, at least about 93%, even more preferably, at least about 95%, and more preferably, at least about 99% homologous to porcine Lhx3a. More preferably, the isolated polypeptide comprising is pLhx3a. Most preferably, the isolated polypeptide comprising a pLhx3a is SEQ ID NO:14.

The invention also includes an isolated polypeptide comprising a mammalian Lhx3. Preferably, the isolated polypeptide comprising a mammalian Lhx3 is at least about 93% homologous to SEQ ID NO:16. More preferably, the isolated polypeptide comprising a mammalian Lhx3 is at least about 94%, more preferably, at least about 95%, and more preferably, at least about 99% homologous to porcine Lhx3b. More preferably, the isolated polypeptide comprising is pLhx3b. Most preferably, the isolated polypeptide comprising a pLhx3b is SEQ ID NO:16.

The invention also includes an isolated polypeptide comprising a mammalian Lhx3. Preferably, the isolated polypeptide comprising a mammalian Lhx3 is at least about 94.5% homologous to SEQ ID NO:8. More preferably, the isolated polypeptide comprising a human Lhx3 is at least about 95%, more preferably, at least about 97%, and even more preferably, at least about 99% homologous to hLhx3. More preferably, the isolated polypeptide comprising is hLhx3. Most preferably, the isolated polypeptide comprising a human Lhx3 is SEQ ID NO:8.

The invention also includes an isolated polypeptide comprising a mammalian Lhx3. Preferably, the isolated polypeptide comprising a mammalian Lhx3 is at least about 91.5% homologous to SEQ ID NO:10. More preferably, the isolated polypeptide comprising a human Lhx3a is at least about 92%, more preferably, at least about 95%, and even more preferably, at least about 99% homologous to hLhx3a. More preferably, the isolated polypeptide comprising is hLhx3a. Most preferably, the isolated polypeptide comprising a human Lhx3a is SEQ ID NO:10.

The invention also includes an isolated polypeptide comprising a mammalian Lhx3. Preferably, the isolated polypeptide comprising a mammalian Lhx3 is at least about 94.5% homologous to SEQ ID NO:12. More preferably, the isolated polypeptide comprising a human Lhx3b is at least about 95%, more preferably, at least about 96%, even more preferably, at least about 97%, and more preferably, at least about 99% homologous to hLhx3b. More preferably, the isolated polypeptide comprising is hLhx3b. Most preferably, the isolated polypeptide comprising a human Lhx3b is SEQ ID NO:12.

The invention also includes an isolated polypeptide comprising a portion of mammalian Lhx3. Preferably, the isolated polypeptide comprising a portion of mammalian Lhx3 is at least about 58% homologous to SEQ ID NO:28. More preferably, the isolated polypeptide comprising a portion of mammalian Lhx3 is at least about 60%, more preferably, at least about 70%, even more preferably, at least about 80%, more preferably, at least about 90%, yet more preferably, at least about 95%, and more preferably, at least about 99% homologous to SEQ ID NO:28. More preferably, the isolated polypeptide comprising a portion of mammalian Lhx3 is a portion of hLhx3a. Most preferably, the isolated polypeptide comprising a portion of hLhx3a is SEQ ID NO:28 (ie., from about amino acid 1 to about amino acid 26).

The invention also includes an isolated polypeptide comprising a portion of mammalian Lhx3. Preferably, the isolated polypeptide comprising a portion of mammalian Lhx3 is at least about 94% homologous to SEQ ID NO:29.

More preferably, the isolated polypeptide comprising a portion of mammalian Lhx3 is at least about 95%, and more preferably, at least about 99% homologous to SEQ ID NO:29. More preferably, the isolated polypeptide comprising a portion of mammalian Lhx3 is a portion of hLhx3b. Most preferably, the isolated polypeptide comprising a portion of hLhx3a is SEQ ID NO:29 (i.e., from about amino acid 1 to about amino acid 31.

The present invention also provides for analogs of proteins or peptides which comprise a mammalian Lhx3 protein as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine;

Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “mutants,” “derivatives,” and “variants” of the peptides of the invention (or of the DNA encoding the same) which mutants, derivatives and variants are Lhx3 peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the Lhx3 peptide of the present invention. A biological property of a Lhx3 includes, but is not limited to include, the ability of the peptide to bind specifically with PGBE, which can be detected by, for example, electrophoretic mobility shift assay (EMSA) as disclosed herein. Further, another biological activity of Lhx3 is the ability to trans-activate a pituitary trophic hormone gene (e.g., αGSU) promoter thereby inducing expression of the gene and/or to activate genes in the nervous system. Moreover, another biological activity of Lhx3 is the ability to trans-activate the gene encoding prolactin. Additionally, the biological activity of Lhx3 includes the ability of the Lhx3 homeodomain to bind with DNA. Moreover, the biological activity of Lhx3 also includes the ability of Lhx3 to synergize with pituitary transcription factors including, but not limited to, pit-1, to induce transcription driven by a specific promoter sequence (e.g., the TSHβ promoter sequence).

Further, the invention should be construed to include naturally occurring variants or recombinantly derived mutants of Lhx3, which variants or mutants render the protein encoded thereby either more, less, or just as biologically active as the full-length proteins of the invention.

In addition to naturally-occurring allelic variants of a peptide molecule of the invention, the skilled artisan will further appreciate that changes can be introduced by mutation of the nucleic acid encoding the protein leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to polypeptides encoded by nucleic acid molecules of the invention, which polypeptides contain changes in amino acid residues that are not essential for activity. Such polypeptides differ in amino acid sequence from any of SEQ ID NOs: 2, 8, 10, 12, 14, and 16, yet retain biological activity.

To generate variant proteins, an isolated nucleic acid molecule encoding a variant protein can be created by introducing one or more nucleotide substitutions, additions or deletions into the nucleotide sequence of any of SEQ ID NOs: 1, 7, 9, 11, 13, 15, and 22, such that one or more amino acid residue substitutions, additions or deletions are introduced into the encoded Lhx3 protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

One skilled in the art would appreciate, based upon the disclosure provided herein, that a mutant polypeptide that is a variant of a polypeptide of the invention can be assayed for: (1) the ability to bind with a nucleic acid that specifically binds with Lhx3 (e.g., pituitary glycoprotein basal element, PGBE); (2) the ability to trans-activate a pituitary trophic hormone gene (e.g., αGSU) promoter; (3) the ability of the Lhx3 homeodomain to bind with DNA; (4) the ability to activate the gene encoding prolactin; (5) the ability to activate genes in the nervous system; (6) the ability to activate the gene encoding Pit-1; and (7) the ability of Lhx3 to synergize with transcription factors including, but not limited to, Pit-1, to induce transcription driven by a specific promoter sequence (e.g., the TSHβ promoter sequence).

The nucleic acids, and peptides encoded thereby, are useful tools for elucidating the function(s) Lhx3 in a cell. Further, they are useful for localizing the nucleic acid, protein, or both, in a cell and for assessing the level of expression of the nucleic acid and/or protein under selected conditions including in response to therapeutic treatment. Further, nucleic and amino acids comprising mammalian Lhx3 are useful diagnostics which can be used, for example, to identify a compound that affects expression of the protein and is a candidate therapeutic for a disease, disorder, or condition associated with altered expression of Lhx3. In addition, the nucleic acids, the proteins encoded thereby, or both, can be administered to a mammal to increase or decrease expression of Lhx3 in the mammal. This can be therapeutic to the mammal if under or over-expression of Lhx3 in the mammal mediates a disease or condition associated with altered expression of the protein compared with normal expression of Lhx3 in a healthy mammal.

V. Antibodies

The invention also includes an antibody specific for a human Lhx3, or a portion thereof.

In one embodiment, the antibody is a rabbit polyclonal antibody to Lhx3a. The antibody can be specific for any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with Lhx3. That is, the invention includes immunizing an animal using an immunogenic portion comprising, for example, amino acid residues 1-26 (i.e., the sequence MLLETGLERDRAROCAAAVCTLGGTR [SEQ ID NO:28]), which is a portion of human Lhx3a (SEQ ID NO:10). In another embodiment, the antibody is a rabbit polyclonal antibody directed to amino acid residues 1-31 of hLhx3b (SEQ ID NO:12), (ie., the sequence MEARGELGPARESAGGDLLLALLARRADLRR [SEQ ID NO:29]).

The invention also includes production of antibodies that specifically bind with mouse Lhx3, including mLhx3a and/or mLhx3b. These antibodies can specifically bind with either one or at least one isoform of mouse Lhx3. Further, the antibodies can specifically bind with Lhx3 from other species, e.g., porcine, human, or both, Lhx3. One skilled in the art would understand, based upon the disclosure provided herein, that the anti-Lhx3 antibodies of the invention can recognize either one or both isoforms of Lhx3 from the other species such that the antibodies can specifically bind with at least one of, for example, mLhx3a, mLhx3b, pLhx3a, pLhx3b, hLhx3a, and hLhx3b.

The antibodies can be produced by immunizing an animal such as, but not limited to, a rabbit or a mouse with a protein of the invention, or a portion thereof, or by immunizing an animal using a protein comprising at least a portion of Lhx3 and a tag polypeptide portion comprising, for example, a maltose binding protein tag polypeptide portion and a portion comprising the respective Lhx3a or b amino acid residues. For instance, the present invention includes, but is not limited to, the immunizing an animal using an immunogenic portion encoded by an isolated nucleic acid (SEQ ID NO:23) comprising from about nucleotide −104 to about nucleotide 78 of SEQ ID NO:9 encoding hLhx3a, and/or an isolated nucleic acid (SEQ ID NO:24) encoding an immunogenic portion comprising from about nucleotide −119 to about nucleotide 93 of SEQ ID NO:11 encoding hLhx3b into an appropriate vector. One skilled in the art would appreciate, based upon the disclosure provided herein, that smaller fragments of these nucleic acids can also be used to produce antibodies that specifically bind Lhx3.

One skilled in the art would appreciate, based upon the disclosure provided herein, that various portions of an isolated Lhx3 polypeptide can be used to generate antibodies to either highly conserved regions of Lhx3 (e.g., LIM domains, homeodomain, and the like) or to non-conserved regions such as, but not limited to, the amino-terminal amino acids of hLhx3a, hLhx3b, or both. The skilled artisan, based upon the disclosure provided herein, would appreciate that the non-conserved regions of a protein of interest can be more immunogenic than the highly conserved regions which are conserved among various organisms. Further, immunization using a non-conserved immunogenic portion can produce antibodies specific for the non-conserved region thereby producing antibodies that do not cross-react with other proteins which can share one or more conserved portions.

One skilled in the art would appreciate, based upon the disclosure provided herein, which portions of Lhx3 are less homologous with other proteins sharing conserved domains (e.g., chicken LIM-3, Xenopus LIM-3, Drosophila LIM-3, zebrafish LIM-3, and the like). Such non-conserved domains include, but are not limited to, the portion comprising the amino acid sequence

| | |
|---|---|
| MLLETELAGDRDRPGAPAAAAVCTLPGTR of porcine Lhx3a | (SEQ ID NO:31); |
| MEARGELGPSRESAGGDLLLALLARREDLRR of porcine Lhx3b | (SEQ ID NO:33); |
| MLLETGLERDRARPGAAAVCTLGGTR of human Lhx3a | (SEQ ID NO:28); and |
| MEARGELGPARESAGGDLLLALLARRADLRR of human Lhx3b | (SEQ ID NO:29). |

However, the present invention is not limited to these domains; instead, the skilled artisan would understand that other non-conserved regions of the Lhx3 proteins of the invention can be used to produce the antibodies of the invention as disclosed herein.

The invention should not be construed as being limited solely to the antibodies disclosed herein or to any particular immunogenic portion of the proteins of the invention. Rather, the invention should be construed to include other antibodies, as that term is defined elsewhere herein, to hLhx3 isoforms, or portions thereof, or to proteins sharing at least about 65% homology with these proteins.

The invention encompasses polyclonal, monoclonal, synthetic antibodies, and the like. One skilled in the art would understand, based upon the disclosure provided herein, that the crucial feature of the antibody of the invention is that the antibody bind specifically with hLhx3. That is, the antibody of the invention recognizes Lhx3 (e.g., Lhx3a, Lhx3b), or a fragment thereof, on Western blots, in immunostaining of cells, and immunoprecipitates Lhx3 using standard methods well-known in the art.

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide can be prepared using any well known monoclonal antibody preparation procedures, such as those described, for example, in Harlow et al., 1988, supra, and in Tuszynski et al. (1988, Blood, 72:109-115), and methods set forth elsewhere herein. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in Wright et al. (supra), and in the references cited therein, and in Gu et al. (1997, Thrombosis and Hematocyst 77:755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruifet al. 1995, J. Mol. Biol. 248:97-105).

VI. Compositions

The invention includes a composition comprising an isolated purified Lhx3, or fragment thereof, and a composition comprising an isolated nucleic acid that specifically binds Lhx3 (e.g., αGSU gene promoter sequence [PGBE], TSHβ gene promoter sequence, Lhx3 consensus binding sequence, Pit-1 promoter sequence, prolactin promoter sequence, and the like). The compositions can be used to assess Lhx3 activity (e.g., the ability of Lhx3 to specifically bind with a nucleic acid that binds with Lhx3).

Further, a composition comprising a nucleic acid that specifically binds with Lhx3 can be administered to a cell, or an animal, to inhibit binding of Lhx3 with a nucleic acid that specifically binds with Lhx3 thereby treating a disease. disorder or condition mediated by altered binding of Lhx3 with a nucleic acid that specifically binds with Lhx3. One skilled in the art would appreciate, based upon the disclosure provided herein, that administering a composition comprising an isolated nucleic acid that specifically binds with Lhx3 can compete with endogenous nucleic acid that otherwise binds with Lhx3 to reduce or inhibit Lhx3/nucleic acid binding interactions associated with a disease, disorder or condition of the pituitary.

The invention includes a composition comprising an isolated purified polypeptide comprising a mammalian Lhx3 (e.g., porcine Lhx3, and human Lhx3, and isoforms thereof). Preferably, the composition comprises a pharmaceutically acceptable carrier. The composition can be administered to a mammal afflicted with a disease, disorder or condition associated with decreased level of Lhx3 compared with the levell of Lhx3 in an otherwise identical mammal not suffering from such disease, disorder or condition.

Additionally, a composition comprising an isolated purified polypeptide comprising a mammalian Lhx3, or an immunogenic portion thereof, can be administered to an animal to induce an immune response thereto. One skilled in the art would appreciate, based upon the disclosure provided herein, that the composition can be used to produce useful antibodies that specifically bind with Lhx3.

The invention further includes a composition comprising an isolated hLhx3a, or a fragment thereof wherein the fragment comprises amino acid residues from about 1 to about 36 (SEQ ID NO:28). Further, the invention includes a composition comprising an isolated hLhx3b, or a fragment thereof wherein the fragment comprises amino acid residues from about 1 to about 31 (SEQ ID NO:29).

The invention further includes a composition comprising an isolated pLhx3a, or a fragment thereof wherein the fragment comprises amino acid residues from about 1 to about 29 (SEQ ID NO:31). Further, the invention includes a composition comprising an isolated pLhx3b, or a fragment thereof wherein the fragment comprises amino acid residues from about 1 to about 31 (SEQ ID NO:33).

Administering Lhx3 is useful since previous studies demonstrate that Lhx3 plays a crucial role in pituitary growth development (see, e.g., studies demonstrating that ablating the Lhx3 gene is lethal in mice) and that Lhx3 is involved in pituitary maintenance and function. Thus, one skilled in the art would understand, based upon the disclosure provided herein, that administration of Lhx3 is an important potential therapeutic for treatment of a disease, disorder or condition mediated by decreased Lhx3 expression, function, or both.

For administration of the above-mentioned compositions to a mammal, a polypeptide, or the nucleic acid encoding it, or both, can be suspended in any pharmaceutically acceptable carrier, for example, HEPES buffered saline at a pH of about 7.8. Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer Lhx3, alone or in combination with a nucleic acid encoding the same.

The invention encompasses the preparation and use of pharmaceutical compositions comprising a compound useful for treatment of any disease, disorder or condition associated with altered expression of Lhx3 in a mammal. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In addition, the administration of the compositions to birds is also contemplated.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxy propyl methyl cellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methyl cellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to. Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, ie., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically dosages of the compound of the invention which may be administered to an animal, preferably a human, range in amount from 1 microgram to about 100 grams per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 milligram to about 10 grams per kilogram of body weight of the animal. More preferably, the dosage will vary from about 10 milligrams to about 1 gram per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

VII. Methods

A. Detection of Lhx3

The instant invention includes novel assays to detect the presence of Lhx3 in a sample and to quantitate the amount of nucleic acid (i.e., RNA, DNA, or both) encoding Lhx3 present in a sample. That is, the invention includes nucleic acid amplification methods such as, but not limited to, PCR-based quantitative sequence detection (QSD), which methods can be used to assess the level of nucleic acid encoding Lhx3 in a sample. Further, the invention includes methods of detecting and/or quantitating the amount of nucleic acid encoding hLhx3a, hLhx3b, or both, present in a sample.

For example, the invention encompasses a PCR-based assay using cDNA, genomic DNA, or both, where the absence or presence of a nucleic acid encoding Lhx3 in a sample can be determined.

One skilled in the art would appreciate, based upon the disclosure provided herein, that the nucleic acid sequence should be sufficiently unique to hLhx3 such that the identical sequence, or a sequence more than 56% homologous to it, does not occur in the sample being tested. By using a sufficiently unique nucleic acid target sequence, any nucleic acid amplification product produced during the amplification phase will not complementary to and, therefore, will not cross-hybridize and/or amplify non-Lhx3 nucleic acids under high stringency conditions such as conditions well-known in the art and set forth in, for example, Sambrook et al. (1989, Molecular Cloning: A Laboratory Manual, Cold Spring Harbor Laboratory Press, New York), Ausubel et al. (1997, Current Protocols in Molecular Biology, Green & Wiley, New York).

PCR is the preferred amplification technique used in the amplification step of the present methods. However, the amplification step may also be carried out using any suitable amplification technique known in the art or to be developed. Suitable amplification techniques are described in the following patents, each of which is incorporated herein by reference as if set forth in its entirety: U.S. Pat. Nos. 4,683,195; 4,683,202; 4,965,188; 5,409,818; 5,437,990; 4,957,858; and PCT Patent Publication No. 89/06995.

As described in U.S. Pat. No. 5,770,368, and in standard treatises such as, for example, Sambrook et al., supra, and Ausubel et al., supra, the preferred PCR amplification procedure used in the present method comprises a target nucleic acid portion unique to a nucleic acid encloding hLhx3 is amplified by treating the double-stranded target polynucleotide with two oligonucleotide primers, each being complementary to one of the two strands of the target. The primers hybridize with their complementary strands and extension products are synthesized using DNA polymerase and at least four deoxyribonucleotide triphosphates (dNTPs). The extension products are separated from their complementary strands by denaturation at an elevated temperature, typically ranging from about 80° C. to about 100° C. The reaction mixture is repeatedly cycled between a low temperature annealing step usually ranging from about 37° C. to about 70° C. during which the primers hybridize to their complementary strands, an intermediate temperature (from about 70° C. to about 80° C.) primer extension step, to the higher temperature denaturation step at a temperature from about 80° C. to about 100° C. These temperature steps, or thermal cycling, are repeated many times, typically about 20 to about 40 cycles are carried out, followed by a final synthesis step at about 70° C. and a 4° C. soak to stop the reaction.

PCR reagents are the chemicals, apart from the target nucleic acid sequence, needed to perform the PCR process. As disclosed by Mayrand (1997, U.S. Pat. No. 5,691,146, these chemicals generally consist of five classes of components: (i) an aqueous buffer, (ii) a water soluble magnesium salt, (iii) at least four deoxyribonucleotide triphosphates (dNTPs) (conventionally, dATP, dTTP, dGTP, dCTP), (iv) oligonucleotide primers (typically two primers for each target sequence, the sequences defining the 5′ ends of the two complementary strands of the double-stranded target sequence), and (v) a polynucleotide polymerase, preferably a DNA polymerase, more preferably a thermostable DNA polymerase, i e., a DNA polymerase which can tolerate temperatures between 90° C. and 100° C. for a total time of at least 10 minutes without losing more than about half its activity.

Primers for the amplification steps are the same if used for a reverse transcription step at the outset to convert RNA into DNA before carrying out the amplification procedure. Preferably, primers are chosen which only amplify target nucleic acid sequences from a nucleic acid encoding hLhx3a, hLhx3b, or both. In the present invention, the primers amplify only a target nucleic acid sequence within the coding region of hLhx3 because they have less than 91% sequence similarity to other sequences present in a sample based on the stringency conditions disclosed herein. Further, by selecting forward primers from the unique amino terminal portion, i.e., 5′ region, of hLhx3a or hLhx3b, the PCR analysis can detect the presence of either isoform, or both, in a sample. Therefore, amplification using the preferred primers does not amplify any non-Lhx3 nucleic acids in mammalian cells such that DNA extracted from mammalian cells following PCR in the absence of hLhx3 template DNA will not yield the hLhx3-specific amplification product specified by these primers.

Preferred primer pairs and probes target the amino-terminal region of the hLhx3 coding sequence. A preferred primer pair that specifically amplifies a nucleic acid encoding

| | |
|---|---|
| hLhx3a (i.e., a forward (i.e., sense) primer | |
| 5′-ATGCTGCTGGAAACGGGGCTCG-3′ | [SEQ ID NO:34] |
| and reverse primer | |
| 5′-CCGAGTCCCGCCCAAGGTGC-3′ | [SEQ ID NO:35]) |

is used for standard PCR and for QSD. The present invention also includes a forward primer that specifically amplifies a nucleic acid encoding hLhx3a comprising the sequence 5′-ggcacgagccccgcacgacg (i.e., nucleic acid from about −104 to about −85 of SEQ ID NO:9). Further, the present invention includes using an internal oligonucleotide probe that is at least about 45% with the sequence between nucleotide −104 and nucleotide 78 of SEQ ID NO:9 (i.e., SEQ ID NO:23).

Another preferred primer pair (SEQ ID NO:36 and SEQ ID NO:37), which specifically amplifies a nucleic acid encoding hLhx3b used for PCR and QSD, is also included in the invention. One skilled in the art would appreciate, based upon the disclosure provided herein, that a primer pair encompassing any sequence between nucleotide −119 and nucleotide 93 of SEQ ID NO:11 can be used to specifically amplify a nucleic acid encoding hLhx3b. Further, the present invention includes using an internal oligonucleotide probe that is at least about 67% with the sequence between nucleotide −119 and nucleotide 93 of SEQ ID NO:11 (i.e., SEQ ID NO:24).

Irrespective of which primer pair is used, an internal oligonucleotide probe (e.g., 5′-GCGACCGAGCGAGGCCCGGGGCCGC-3′[SEQ ID NO:38]), which hybridizes with a nucleic acid sequence that lies between the forward and reverse primer sequences can be used to confirm the identity of the amplification product by PCR and/or QSD.

Alternatively, one skilled in the art would appreciate, based upon the disclosure provided herein, that the internal oligonucleotide probe can comprise any region within the portion of the nucleic acid encoding a non-conserved portion of hLhx3a or hLhx3b being amplified using any given primer pair. For example, an internal oligonucleotide probe specific for detecting a nucleic acid encoding hLhx3a can have the sequence 5′-GCGACCGAGCGAGGCCCGGGGCCGC-3′ (SEQ ID NO:38). Similarly, an internal oligonucleotide probe specific for detecting a nucleic acid encoding hLhx3b can have the sequence 5′-CCCGGCCCGGGAGTCGGCGGGAGGC-3′ (SEQ ID NO:39). However, the skilled artisan would appreciate, based upon the disclosure provided herein, that the sequence of the internal oligonucleotide probe used to detect either hLhx3a or hLhx3b is not limited to any specific sequences exemplified herein. Thus, once armed with the sequences of Lhx3 disclosed herein, one skilled in the art would be able to design appropriate primer pairs and internal probe sequences following methods well-known in the art and taught elsewhere herein.

It will be understood by those skilled in the art based on this disclosure that in the present invention, the target nucleic acid and the portion of the amplified target sequence to which the PCR oligonucleotide probe hybridizes are unique to hLhx3a, hLhx3b, or both, such that the probe and primers do not hybridize to non-Lhx3 nucleic acids under conditions of high stringency. Thus, the nucleic acid-based detection method of the present invention only detects amplification of the specific, unique hLhx3 target nucleic acid sequence and not that of other sequences which may be present in the sample.

In the standard PCR assay, the amplified target nucleic acid sequence can be detected directly by any method that can distinguish among the different lengths of DNA. Electrophoresis through agarose gels is the standard method known in the art for use in separating, identifying, and purifying DNA fragments following PCR. The location of the DNA within the gel can be determined directly by staining the gel with low concentrations of the intercalating fluorescent dye, ethidium bromide (EtBr). Band(s) corresponding to the predicted length for the amplified target DNA can then be detected by direct examination of the gel in ultraviolet light.

Additionally, the DNA bands from an electrophoresed sample can be probed by Southern blotting using a single-stranded oligonucleotide probe which is complementary to a sequence located between the two selected oligonucleotide primers in the amplified target nucleic acid sequence. Usually, the oligonucleotide probe is labeled with a radioactive or fluorescent tag, or attached directly or indirectly to an enzyme molecule such that the probe specifically bound to the immobilized complementary target sequence may be localized.

In the preferred embodiment herein, the oligonucleotide probe was complementary to a specific portion of the hLhx3 coding sequence. However, the present invention is not limited to this sequence. Rather, the oligonucleotide probe can be selected to hybridize to any target amplified nucleic acid located between two primer pairs all of which hybridize to a sequence in hLhx3 but which do not hybridize to a nucleic acid that does not encode hLhx3 that can be present in a sample of interest.

The oligonucleotides used in the invention can be synthesized using any standard method known or to be developed. Suitable syntheses are described in Ozaki et al. (1992, Nucleic Acids Res. 20:5205-5214) and Agrawal et al. (1990, Nucleic Acids Res. 18:5419-5423).

The oligonucleotide probes of the invention are preferably conveniently synthesized on an automated DNA synthesizer such as a Perkin-Elmer (Foster City, Calif.) Model 392 or 394 DNA/RNA synthesizer using standard chemistries, such as phosphoramidite chemistry described in Beaucage and Iyer (1992, Tetrahedron 48:2223-2311), Molko et al. (U.S. Pat. No. 4,980,460), Koster et al. (U.S. Pat. No. 4,725,677), Caruthers et al. (U.S. Pat. Nos. 4,415,732 and 4,458,066). However, other similar syntheses using chemistries and techniques may be used. Alternative chemistries resulting in non-natural backbone groups, such as phosphorothioate, phosphoramidate, and the like, may also be used provided the hybridization efficiencies of the resulting oligonucleotides are not adversely affected.

Preferably, the oligonucleotide probe is in the range of about 15 to about 150 nucleotides in length. The precise sequence and length of an oligonucleotide probe of the invention depends in part on the nature of the target nucleic acid sequence to which it hybridizes. The binding location and length may be varied to achieve appropriate annealing and melting properties for a particular embodiment by one skilled in the art in accordance with known techniques such as “Taqman”-type assays.

Oligonucleotides of the present invention include linear oligomers of natural or modified monomers or linkages, such as deoxyribonucleotides, ribonucleotides, and the like, which are capable of specifically binding to a target polynucleotide by way of a regular pattern of monomer-to-monomer interactions, such as Watson-Crick base pairing. Usually, monomers are linked by phosphodiester bonds or their analogs to form oligonucleotides ranging in size from a few monomeric units, e.g., 3-4, to several tens of monomeric units. Whenever an oligonucleotide is represented by a sequence of letters, such as “ATGCCTG,” it will be understood that the nucleotides are in a 5′→3′ order from left to right and that “A” denotes deoxyadenosine, “C” denotes deoxycytidine, “G” denotes deoxyguanosine, and “T” denotes thymidine, unless otherwise noted. Analogs of phosphodiester linkages include phosphorothioate, phosphoranilidate, phosphoramidate, and similar compounds.

In a preferred embodiment, amplification of the hLhx3-specific target sequence specified by the primer pair is detected by QSD (also referred to herein as “real time PCR”). Preferably, a Model 7700 Sequence Detector laser fluorometer/thermal cycler is used for the QSD procedure to detect the fluorescence of the PCR sample mixture before and after each round of amplification. Such a QSD procedure is described in Heid et al. (1996, Genome Res. 6:986-994).

In another important aspect of the oligonucleotide probes of the present invention, the probes used for QSD include fluorescer and quencher molecules attached to the oligonucleotide. As used herein, the terms “quenching” and “fluorescence energy transfer” refer to the process whereby when a fluorescer molecule and a quencher molecule are in close proximity, whenever the fluorescer molecule is excited by an energy source, a substantial portion of the energy of the excited state nonradiatively transfers to the quencher where it either dissipates nonradiatively or is emitted at a different emission wavelength than that of the fluorescer.

It is well known that the efficiency of quenching is a strong function of the proximity of the fluorescer and the quencher, i.e., as the two molecules get closer, the quenching efficiency increases. As quenching is strongly dependent on the physical proximity of the reporter molecule and quencher molecule, it has been assumed that the quencher and reporter molecules must be attached to the probe within a few nucleotides of one another, usually with a separation of about 6-16 nucleotides (e.g., Lee et al., 1993, Nucleic Acids Res. 21:3761-3766). Typically, this separation is achieved by attaching one member of a reporter-quencher pair to the 5′ end of the probe and the other member to a base 6-16 nucleotides away.

Preferably, fluorescer molecules are fluorescent organic dyes derivatized for attachment to the terminal 3′ carbon or terminal 5′ carbon of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally, whether the quencher molecule is fluorescent or simply releases the transferred energy from the fluorescer by non-radiative decay, the absorption band of the quencher should substantially overlap the fluorescent emission band of the fluorescer molecule. Non-fluorescent quencher molecules that absorb energy from excited fluorescer molecules, but which do not release the energy radiatively, are referred to herein as chromogenic molecules.

Selection of appropriate fluorescer-quencher pairs for particular probes may be undertaken in accordance with known techniques such as those of, for example, Clegg et al., 1993, Proc. Natl. Acad. Sci USA 90:2994-2998; Wu et al., 1994, Anal. Biochem. 218:1-13; Pesce et al., 1971, In: Fluorescence Spectroscopy, Marcel Dekker, New York; and White et al., 1970, In: Fluorescence Analysis: A Practical Approach, Marcel Dekker, New York. Fluorescent and chromogenic molecules and their relevant optical properties from which exemplary fluorescer-quencher pairs may be selected are listed and described in, e.g., Berlman (1971, In: Handbook of Fluorescence Sprectra of Aromatic Molecules, 2nd ed., Academic Press, New York). Examples of derivatizing fluorescer and quencher molecules for covalent attachment via common reactive groups that can be added to an oligonucleotide in the present invention may be found in, e.g., Haugland, 1992, In: Handbook of Fluorescent Probes and Research Chemicals, Molecular Probes, Eugene, Oreg.; U.S. Pat. No. 3,996,345; and U.S. Pat. No. 4,351,760).

Preferred fluorescer-quencher pairs are xanthene dyes, including fluoresceins, and rhodamine dyes. Many suitable forms of these compounds are available commercially with substituents on their phenyl moieties which can be used as the site for bonding or as the bonding functionality for attachment to an oligonucleotide. Another preferred group of fluorescent compounds are the naphthylamines, having an amino group in the alpha or beta position. Included among such naphthylamino compounds are 1-demethylaminonaphthyl-5 sulfonate, 1-anilino-8-naphthalene sulfonate and 2-p-touidinyl-6-naphthalene sulfonate. Other dyes include 3-phenyl-7-isocyanatocoumarin, acridines, such as 9-isothiocyanatoacridine acridine orange; N-(p-(2-benzoxazolyl) phenyl)maleimide; benzoxadiazoles, stilbenes, pyrenes, and the like.

Most preferably, fluorescer and quencher molecules are selected from fluorescein and rhodamine dyes. These dyes and appropriate linking methodologies for attachment to oligonucleotides are well known in the art, and are described in U.S. Pat. No. 4,351,760; Marshall, 1975, Histochemical J. 7: 299-303; U.S. Pat. No. 5,188,934; European Patent Application 87310256.0; and International Application No. PCT/US90/05565, each of which is incorporated by reference herein as if set forth in its entirety.

In a preferred embodiment, amplification of the hLhx3a or hLhx3b-specific target sequence specified by the primer pair is detected by QSD.

There are many linking moieties and methodologies for attaching fluorescer or quencher molecules to the 5′ or 3′ termini of oligonucleotides well known in the art and described, for example, in Eckstein, 1991, In: Oligonucleotides and Analogues: A Practical Approach, IRL Press, Oxford.

Preferably, commercially available linking moieties are employed that can be attached to an oligonucleotide during synthesis. Suitable moieties are available from ClonTech Laboratories (Palo Alto, Calif.).

Rhodamine and fluorescein dyes are also conveniently attached to the 5′ hydroxyl of an oligonucleotide at the conclusion of solid phase synthesis by way of dyes derivatized with a phosphoramidite moiety as described in, for example, U.S. Pat. Nos. 5,231,191 and 4,997,928.

In a preferred embodiment, the oligonucleotide probe includes the fluorescer molecule 6-carboxyfluorescein at its 5′ end and the quencher molecule 6-carboxytetramethylrhodamine attached to the 3′ end of the oligonucleotide probe. The fluorescer and quencher molecules are attached to the oligonucleotide probe as described in Livak et al. (1995, Guidelines for Designing TaqMan™ Fluorogenic Probes for 5′ Nuclease Assays, In: Perkin Elmer Research News, Applied Biosystems Division, Foster City, Calif.). However, it will be understood based on this disclosure that the invention is not limited to this particular fluorescer-quencher pair or to the particular linkages used to attach the molecules to the probe. Rather, as previously discussed herein, a wide variety of fluorescer-quencher molecules may be attached to the oligonucleotide by a variety of linkages. Further, the fluorescer-quencher pair need not be located on nucleotides which are immediately adjacent; instead, the quencher dye may be attached to any nucleotide on a probe and still quench the fluorescence emission of a reporter dye attached to the 5′ end (see Livak et al., supra).

One skilled in art of producing “Taqman” type probes would appreciate that probes should be of sufficient length to assure good hybridization and specificity of binding. Further, if possible, the GC content of the fluorogenic probe should range from about 40% to about 60%. Also, as described in Livak et al., supra, both PCR primers and the fluorogenic probe must hybridize to the target template strands during the annealing step of the PCR amplification. Because the fluorogenic probe is not extended at the 3′ end, the template-probe hybridization is not stabilized by the DNA polymerase extension step which does stabilize the primer-template hybridization by extending from the 3′ end of the primer. Thus, the Tₘ of the probe-template hybrid must be higher than the primer-template hybrid Tₘ. Moreover, the Tm of the probe should be higher than the annealing temperature used in the PCR.

In one preferred embodiment, the fluorescer molecule is released by the 5′→3′ exonuclease activity of the Taq DNA polymerase during amplification thereby causing the separation of the fluorescer and quencher such that the fluorescence level of the sample is increased but leaving the fluorescence of the quencher molecule essentially unchanged such that it serves as an internal standard. However, the invention should not be construed to be limited to the release of a fluorescer from the oligonucleotide probe in order to cause fluorescence of a sample as the target DNA is amplified. Rather, one skilled in the art would recognize that the essential feature of the present invention is the differential quenching of the reporter fluorescer molecule as a result of synthesis of the target nucleic acid sequence.

As described in U.S. Pat. No. 5,691,146, QSD may be performed using oligonucleotide probes which, when present in the single-stranded state in solution, are configured such that the fluorescer and quencher are sufficiently close to substantially quench the reporter fluorescer's emission. However, upon hybridization of the intact quencher-fluorescer oligonucleotide probe with the amplified target nucleic acid sequence, the fluorescer and quencher molecules come to be sufficiently distant from each other. As a result, the quenching is substantially abated causing an increase in the fluorescence emission detected. The QSD of the present invention includes differential quenching of the reporter fluorescer molecule due to the interaction of the fluorescer-quencher probe with the amplified target nucleic acid sequence. The precise mechanism by which the quencher-fluorescer molecules are brought together or taken apart may vary. Guidelines for designing, producing, and using appropriate fluorescer-quencher oligonucleotide probes are known in the art and are described in the above-cited references including, for example, Livak et al. (1995, Guidelines for Designing TaqMan™ Fluorogenic Probes for 5′ Nuclease Assays, In: Perkin Elmer Research News, Applied Biosystems Division, Foster City, Calif.).

The 3′ terminal nucleotide of the oligonucleotide probe may be rendered incapable of extension by a nucleic acid polymerase in one embodiment of the invention. Such blocking may be carried out by the attachment of a fluorescer or quencher molecule to the terminal 3′ carbon of the oligonucleotide probe by a linking moiety, or by making the 3′-terminal nucleotide a dideoxynucleotide. Alternatively, the 3′ end of the oligonucleotide probe may be rendered impervious to the 3′→5′ extension activity of a polymerase by incorporating one or more modified internucleotide linkages onto the 3′ end of the oligonucleotide. Minimally, the 3′-terminal internucleotide linkage must be modified, however, additional the internucleotide linkages may be modified. It is preferred that the 5′→3′ exonuclease ability of the DNA polymerase to cleave off the 5′ nucleotide to which the fluorescer molecule is attached is preserved.

Internucleotide modifications which prevent elongation from the 3′ end of the oligonucleotide probe and/or which block the 3′→5′ exonuclease activity of the DNA polymerase during PCR may include phosphorothioate linkages, methylyphosphonate linkages, boranophosphate linkages, and other similar polymerase-resistant internucleotide linkages. An alternative method to block 3′ extension of the probe is to form an adduct at the 3′ end of the probe using mitomycin C or other like antitumor antibiotics such as those of Basu et al., 1993, Biochemistry 32:4708-4718). Thus, the precise mechanism by which the 3′ end of the oligonucleotide probe is protected from cleavage is not essential so long as the quencher molecule is not cleaved from the oligonucleotide probe.

The level of fluorescence is measured is preferably measured using a laser/fluorometer such as, for example, a ABI Prism Model 7700 Sequence Detector. However, similar detection systems for measuring the level of fluorescence in a sample are included in the invention.

Briefly, QSD is similar to standard PCR assays in that DNA is used as a DNA template to generate millions of copies of the target DNA by Thermus aquaticus (Taq) DNA polymerase enzyme and thermal cycling. However, QSD differs greatly from PCR in that QSD involves the detection of the hybridization of a nonextendible internal fluorogenic quencher-fluorescer DNA probe (e.g., a TaqMan™ probe, Perkin Elmer, Foster City, Calif.) which contains a fluorescer molecule at one end and a quencher molecule on the other end and which is specific for the target DNA sequence being amplified as described in Heid et al. (1996, Genome Res. 6:986-994). When the probe is intact, fluorescent energy transfer occurs and the reporter dye fluorescence is absorbed by the quenching dye (id. at 987). During the extension phase of the PCR cycle, the fluorescent hybridization probe is cleaved by the 5′→3′ exonuclease activity of the DNA polymerase. Once the probe is cleaved, the reporter dye emission is no longer quenched resulting in an increase of the reporter dye fluorescence emission spectra after each round of replication.

Further, the model 7700 sequence detector measures the intensity of the quenching dye emission, which changes very little over the course of the PCR reaction, and uses this measure as an internal standard with which to normalize the reporter dye emission variations. Moreover, the software calculates the threshold cycle (C_{T}) which is the cycle number at which the change in normalized reporter signal (ΔRₙ) crosses a selected threshold point. The change in normalized reporter signal is also calculated by the software by measuring the emission intensity of the reporter divided by the emission of the quencher in a reaction tube and subtracting the same value obtained prior to PCR amplification in that same reaction tube. As demonstrated herein, the threshold cycle is a function of the starting quantity of target DNA and, thus, the point at which the amplification plot crosses the threshold is predictive of the quantity of input target. By determining the threshold cycle for reference standards having known amounts of nucleic acid encoding hLhx3, a reference standard may be developed which enables the quantitation of nucleic acid encoding hLhx3 in unknown samples. The data disclosed demonstrate that quantitative sequence detection provides a simple, efficient, closed-tube assay which is both sensitive and quantitative for hLhx3 in a test sample.

Accordingly, unlike ethidium bromide staining of gel fragments, QSD fluorescence is directly proportional to the amount of fluorescer-quencher probe bound to the specific target DNA in the sample. Thus, by proper data analysis included with the ABI Prism Model 7700 Sequence Detector (Perkin-Elmer Applied Biosystems, Foster City, Calif.), the amount of amplified hLhx3-specific target DNA in the sample may be deduced based upon the level of fluorescence detected in the sample and used to generate a standard curve to quantitate the amount of nucleic acid encoding hLhx3 in any given sample. Further, the data disclosed herein demonstrate that by using forward primers specific for hLhx3a or hLhx3b, the amount of nucleic acid encoding each isoform present in a sample can be assessed.

The invention also includes an isolated nucleic acid that specifically binds with the 5′ end of hLhx3 (e.g., hLhx3a, hLhx3b, or both). The nucleic acid comprises a primer pair which amplifies a unique target sequence of the human genome encoding hLhx3a, hLhx3b, or both.

In a preferred embodiment, the target hLhx3 nucleic acid is the sequence encoding the amino terminal portion of hLhx3a (from about nucleotide −104 to about nucleotide 78 of SEQ ID NO:9) and the forward and reverse primers have the sequences SEQ ID NO:34 and SEQ ID NO:35, respectively. Further, the internal oligonucleotide probe used to detect the amplified nucleic acid has the sequence of SEQ ID NO:38.

In another preferred embodiment, the target hLhx3 nucleic acid is the sequence encoding the amino terminal portion of hLhx3b (from about nucleotide −119 to about nucleotide 93 of SEQ ID NO:11) and the forward and reverse primers have the sequences SEQ ID NO:36 and SEQ ID NO:37, respectively. Further, the internal oligonucleotide probe used to detect the amplified nucleic acid has the sequence of SEQ ID NO:39.

One skilled in the art would understand, based upon the disclosure provided herein, that the present invention is not limited to these primers or oligonucleotide probe sequences. Rather, any oligonucleotide probe having a sequence between the two primers spanning from nucleotide residue −104 up to and including nucleotide residue 78 of a nucleic acid having the sequence SEQ ID NO:9 (hLhx3a), and/or from nucleotide residue −119 up to and including nucleotide residue 93 of a nucleic acid having the sequence SEQ ID NO:11 (hLhx3b), is encompassed in the invention.

B. Methods of Identifying Useful Compounds

The present invention further includes a method of identifying a compound which affects expression of Lhx3 in a cell. The method comprises contacting a cell with a test compound and comparing the level of expression of Lhx3 in the cell so contacted with the level of expression of Lhx3 in an otherwise identical cell not contacted with the compound. If the level of expression of Lhx3 is higher or lower in the cell contacted with the test compound compared with the level of expression of Lhx3 in the otherwise identical cell not contacted with the test compound, this is an indication that the test compound affects expression of Lhx3 in a cell.

Given the importance of Lhx3 in pituitary development and function, a normal level of expression of this protein is crucial to organogenesis, function, and maintenance of this organ since Lhx3 plays an important role in these functions and an appropriate amount of this protein is required for the protein to perform its requisite function(s). For instance, Lhx3 serves an essential role in control of pituitary hormone gene expression. Compounds that affect expression of Lhx3 can therefore have an important impact in development of therapeutics for pituitary associated diseases, disorders or conditions mediated by altered Lhx3 expression. Thus, one skilled in the art would understand, based upon the disclosure provided herein, that the level of Lhx3 expression in a tissue sample obtained from an animal exhibiting normal pituitary development and/or function can be determined and compared with the level of Lhx3 expression in a test sample.

One skilled in the art would appreciate based on the disclosure provided herein that the level of expression of Lhx3 (e.g., hLhx3a, hLhx3b, and the like) in the cell can be assessed by determining the level of expression of mRNA encoding hLhx3 or by using immunological methods to assess hLhx3 expression, and comparing the level of hLhx3 mRNA expression in a cell contacted with the compound with the level of expression in a cell not contacted with the compound. Alternatively, expression of hLhx3 can be assessed immunologically using an antibody that binds specifically with hLhx3 as described elsewhere herein.

The present invention also provides a method of identifying a compound which affects the level of expression of hLhx3a but which does not affect the level of expression of hLhx3b in a cell. The method comprises contacting a cell with a test compound and comparing the level of expression of hLhx3a in the cell with the level of expression of hLhx3a in the same cell prior to or in the absence of contacting the cell with the test compound. Similarly, the level of expression of hLhx3b in an otherwise identical cell contacted with the test compound is compared to the level of expression of hLhx3b in the cell prior to or in the absence of the test compound. As discussed elsewhere herein, the level of hLhx3a expression can be distinguished from the level of hLhx3b by use of hLhx3b-specific and hLhx3a-specific probes such as, for example, specific nucleic acid probes and antibodies.

If the level of expression of hLhx3a in the cell contacted with the test compound is higher or lower than the level of expression of hLhx3a in the same cell prior to or in the absence of contacting the cell with the test compound, and if the level of expression of hLhx3b in the otherwise identical cell contacted with the test compound is not different than the level of expression of hLhx3b in the cell prior to or in the absence of the test compound, this is an indication that the test compound affects the level of expression of hLhx3a but does not affect the level of expression of hLhx3b in a cell.

Although separate but otherwise identical cells are contacted with the test compound in the above assay, the present invention should not be construed to be limited to comparing the effects of a compound on hLhx3b and hLhx3a expression in separate cells. Instead, the present invention is intended to include assays wherein the level of expression of hLhx3b and the level of expression of hLhx3a are determined within the same cell contacted with a compound and compared with the levels of expression of these enzymes in an otherwise identical cell not contacted with the compound. The levels of hLhx3b and hLhx3a can be distinguished by the use of nucleic acid and antibody probes specific for each enzyme which do not cross-react with each other such as, for example, the nucleic acid probes of the invention wherein hLhx3a and hLhx3b-specific PCR primer pairs are disclosed which can be used distinguish between the two isoforms.

Further, one skilled in the art would appreciate based on the disclosure provided herein that, as disclosed in the examples below, a cell that lacks endogenous detectable Lhx3 activity can be transfected with a vector comprising hLhx3a or hLhx3b wherein the expression of hLhx3a, hLhx3b, or both, is effected in the cell. The transfected cell is then contacted with the test compound thereby allowing the determination of whether the compound affects the expression of hLhx3a, hLhx3b, neither, or both proteins. That is, if the cell transfected with and expressing hLhx3a contacted with a test compound exhibits a higher or lower level of expression of hLhx3a compared to the level of expression of hLhx3a in an otherwise identical cell not contacted with the test compound, this is an indication that the test compound affects expression of hLhx3a in a cell. Further, if the cell transfected with hLhx3b and contacted with the test compound exhibits a higher or lower level of expression of hLhx3b compared to an otherwise identical cell not contacted with the test compound, this is an indication that the test compound affects the level of expression of hLhx3b in a cell. This further demonstrates that the test compound affects the level of expression of hLhx3a and hLhx3b in a cell. Additionally, the results may be further quantitated to determine whether the test compound affects the level of expression of hLhx3b to a greater, lesser, or the same, degree as it affects the level of expression of hLhx3a in a cell.

Therefore, one skilled in the art armed with the present invention would be able to, by selectively transfecting a cell lacking detectable levels of hLhx3 expression or hLhx3 activity with either hLhx3b or hLhx3a-expressing vectors, identify a compound which selectively affects hLhx3a but not hLhx3b expression and/or activity as described in the previous assay.

One skilled in the art would appreciate, based upon the disclosure provided herein, that a method of detecting the level of expression, activity, or both, of Lhx3 disclosed herein can also be performed in a cell expressing endogenous Lhx3 such as, but not limited to, a naturally-occurring pituitary cell.

Further, the present invention should also be interpreted to include an assay whereby a cell lacking hLhx3 expression and/or activity is co-transfected with expression vectors encoding hLhx3b and hLhx3a, either separately or on the same vector. The level of hLhx3b and hLhx3a in the cell is measured. Subsequently, the cell is contacted with a test compound and the level of hLhx3b and hLhx3a in the cell is compared to the level of these isoforms in an otherwise identical cell which is not contacted with the test compound. The level of hLhx3b can be differentiated from the level of hLhx3a by the use of specific nucleic acid probes and/or antibodies which do not cross-react between the two isoforms.

The data disclose herein demonstrate that the two hLhx3 isoforms have different abilities to bind nucleic acids and to trans-activate specific pituitary hormone genes. Therefore, assays distinguishing between isoforms are useful, for example, to distinguish whether a disease, disorder or condition of the pituitary is mediated by differential expression of the isoforms. Without wishing to be bound by any particular theory, an assay distinguishing between isoforms can be used to assess pituitary tumor formation, nervous system tumor formation, or other pituitary or nervous system disease, since differential expression of Lhx3 isoforms may be associated with such diseases, disorders or conditions.

Additionally, a cell which is not transfected with a vector encoding hLhx3b and/or hLhx3a which cell has endogenous Lhx3 activity, may also be tested for Lhx3 activity and may also be used to detect hLhx3a mRNA expression by, for example, Northern blotting, or to detect hLhx3a protein using an anti- hLhx3a specific antibody of the invention, in order to detect and/or quantify the level of expression of hLhx3a in a cell contacted with a test compound.

The invention farther includes a method of identifying a compound which affects the level of expression of hLhx3b but not the level of expression of hLhx3a. The method comprises contacting a cell with a test compound and measuring the level of hLhx3a expression in the cell and comparing the level of hLhx3a expression in an otherwise identical cell not contacted with the test compound and comparing the level of hLhx3b expression in the same cell or in an otherwise identical cell contacted with the test compound with the level of hLhx3b expression in the same cell or in an otherwise identical cell not contacted with the test compound.

By comparing the level of expression of hLhx3b and hLhx3a in these cells, it may be determined whether contacting the cell a with a test compound affects: the level of expression of both hLhx3b and hLhx3a; neither the level of hLhx3b nor hLhx3a; the level of hLhx3b only; the level of hLhx3a only; or the level of both hLhx3b and hLhx3a but to a different degree. For example, if the level of hLhx3b is higher or lower in a cell contacted with the test compound compared to the level of hLhx3b activity in an otherwise identical cell not contacted with the test compound, but the level of hLhx3a in a cell contacted with the test compound is not changed compared with the level of hLhx3a in an otherwise identical cell not contacted with the test compound, this result is an indication that the test compound affects the level of hLhx3b but does not affect the level of hLhx3a in a cell. Further, the various permutations of the possible assays using cells which are either untransfected, singly transfected with either hLhx3b or hLhx3 a-expressing vectors, or cotransfected with both hLhx3b- and hLhx3a-expressing vectors have been described previously elsewhere herein and apply to this assay as well.

The invention also includes an additional method of identifying a compound which affects the level of activity of hLhx3a but does not affect the level of hLhx3b activity in a cell. The method comprises transfecting a cell which does not exhibit detectable endogenous Lhx3 activity, with a vector encoding hLhx3b and, similarly, transfecting an otherwise identical cell with a vector encoding hLhx3a. The activity of the respective Lhx3 enzyme is measured in each cell in the absence or presence of contacting the cell with a test compound. If the level of hLhx3a in a cell contacted with the test compound is higher or lower than the level of hLhx3a in an otherwise identical cell which is not contacted with the test compound, then the result indicates that the test compound affects the level of hLhx3a in a cell. Further, if a the level of hLhx3b in a cell contacted with the test compound does not change compared with the level of hLhx3b in an otherwise identical cell not contacted with the test compound, these results indicate that the test compound affects the level of hLhx3a activity in a cell but does not affect the level of hLhx3b activity in a cell. Thus, this assay allows the identification of a compound which affects the level of activity of one Lhx3 isoform but not the level of another Lhx3 isoform by comparing the effect(s) of the compound on each Lhx3 enzyme in a cell.

Additionally, the invention includes a method of affecting the level of expression of hLhx3a in a cell. The method comprises introducing into a cell, which may or may not have endogenous Lhx3 activity, an isolated nucleic acid encoding hLhx3a. The nucleic acid further comprises a promoter/regulatory region driving the expression of hLhx3a in the cell. The level of expression of hLhx3a in the cell is measured and is compared with the level of hLhx3a in an otherwise identical cell that does not comprise the isolated nucleic acid encoding hLhx3a. Introduction of the nucleic acid encoding hLhx3a into the cell causes the expression of the nucleic acid encoded by the vector in the cell thereby affecting the level of expression of hLhx3a in the cell relative to a cell which does not comprise the isolated nucleic acid. This method allows the evaluation of various vectors and nucleic acid sequences that encode and express Lhx3 in a cell. Further, the transfected cell expressing Lhx3 can be used as a model system useful for the evaluation of compounds that affect the level of Lhx3 expression in a cell.

In one embodiment, the nucleic acid encoding hLhx3a is transfected into a cell using a plasmid vector thereby causing an increase level of hLhx3a expression in the transfected cell compared with the level of hLhx3a expression in an otherwise identical cell which is not transfected with the vector. Thus, the level of hLhx3a in the transfected cell is higher than the level of hLhx3a in an otherwise identical cell which is not transfected thereby increasing the level of hLhx3a in a cell, thereby affecting the level of expression of hLhx3 a in a cell.

The invention also includes a method of inhibiting the level of expression of hLhx3a in a cell. The method comprises introducing an isolated antisense nucleic acid complementary to a nucleic acid encoding hLhx3a into a cell. The isolated antisense nucleic acid further comprises a promoter/regulatory region driving the expression of the antisense nucleic acid in the cell. Expression of the isolated nucleic acid encoding hLhx3a in an antisense orientation in the cell inhibits the expression of hLhx3a in the cell. Therefore, the level of hLhx3a in the cell comprising the isolated antisense nucleic acid is lower than the level of hLhx3a in an otherwise identical cell which does not comprise the isolated antisense nucleic acid, such that the introduction of the isolated antisense nucleic acid into a cell decreases the level of hLhx3a in a cell.

Inhibiting or decreasing the level of expression of hLhx3a in a cell is useful for treating a disease, disorder, or condition mediated by an increased level of expression of hLhx3a compared with normal level of hLhx3a expression. This is due to the fact that hLhx3 expression, including expression of hLhx3 isoforms detected in cells as disclosed herein, is crucial to normal pituitary development, function, and maintenance as demonstrate by gene ablation studies in mice. Thus, increased level of hLhx3a expression can be deleterious such that inhibiting such expression would provide a useful therapeutic effect in a cell over-expressing hLhx3a where such over-expression compared with normal level of expression is associated with a disease, disorder or condition of the pituitary.

Additionally, the level of Lhx3 in a cell can be affected by introducing into the cell antibodies, including those of the invention, that specifically bind the protein. Methods of inhibiting cell expression using antibodies are well known in the art. For example, the antibodies can be microinjected into the cell thereby causing the antibodies to bind with hLhx3b and/or hLhx3a thus inhibiting the function of the protein in the cell. Alternatively, a vector comprising nucleic acid encoding the antibody can be introduced into the cell such that the antibody is expressed in the cell thereby binding with Lhx3 and thus preventing Lhx3 function. Further, the nucleic acid encoding the antibody of interest can be operably linked to an appropriate promoter/regulatory sequence such that the expression of the antibody within the cell can be regulated as desired. Therefore, the invention includes various methods of affecting, e.g., inhibiting, the expression of hLhx3b and/or hLhx3a including, but not limited to, the use of antisense nucleic acids and antibodies that specifically bind hLhx3b and/or hLhx3a, introduction of an inhibitory protein molecule that specifically binds with a least one Lhx3 isoform, introduction of a small molecule that specifically binds with Lhx3 isoforms, and the like.

The invention also includes a method of detecting the level of expression of hLhx3b in a biological sample. The method comprises contacting the sample, or a portion thereof, with an hLhx3b-binding molecule. The hLhx3b-binding molecule specifically binds to hLhx3b present in the sample, and any hLhx3b-binding molecule not bound to hLhx3b is removed from the mixture. The hLhx3b-binding molecule which remains bound to the hLhx3b is then detected by standard methods.

In one aspect, the hLhx3b-binding molecule is an antibody molecule which specifically binds to hLhx3b but which does not bind to hLhx3a. The antibody may be used to probe a Western blot of proteins present in a biological sample, such as serum or tissue, comprising hLhx3b. The Western blot is then washed to remove any antibodies which are not specifically bound to hLhx3b and the bound antibodies are visualized by incubating the blot in a secondary antibody conjugated to a detector molecule. Further, the antibody can be used in an ELISA-based assay to quantitate the amount of hLhx3b present in a sample using standard methods well-known in the art.

In one embodiment, the hLhx3b-binding molecule is rabbit polyclonal anti-hLhx3b antibody and the secondary antibody is goat anti-rabbit conjugated to an enzyme such as horse radish peroxidase or alkaline phosphatase. Alternatively, the hLhx3b-binding molecule may be labeled directly such that the molecule may be detected directly.

In another aspect, the hLhx3b-binding molecule is a nucleic acid complementary to at least a portion of a nucleic acid encoding hLhx3b and sharing at least about 67% homology with hLhx3b (SEQ ID NO:24). Thus, the present invention includes detection of the level of expression of hLhx3b in a sample by detecting the presence of a nucleic acid encoding hLhx3b in the sample. For instance, RNA encoding hLhx3b may be detected by probing a Northern blot with a nucleic acid encoding the 5′ portion (i.e., up to about the first 31 amino acids of hLhx3b) of hLhx3b. Any nucleic acid not specifically bound to the blot is removed by washing the filter and any nucleic acid specifically bound thereto is detected by a standard nucleic acid detection method such as those well known in the art using stringency conditions comprising washing the blot at 50° C. using 2× SSC. However, the invention is not to be construed to be limited to any particular method of detecting the hLhx3b-binding molecule.

The amount of hLhx3b or nucleic acid encoding hLhx3b present in a sample is correlated to the level of expression of hLhx3b in the sample. Thus, by detecting the hLhx3b-binding molecule specifically bound to the hLhx3b present in a sample, the level of expression of hLhx3b in the sample may be determined. Methods of detecting the level of expression of a protein and/or nucleic acid encoding a protein are well-known in the art and include, but are not limited to, PCR, real-time PCR, Northern blotting, RNA dot blotting, RNase protection assay, and in situ hybridization, among others. Methods of detecting a protein include, but are not limited to, immunocytochemistry, ELISA, immunoradiometric assay, Western blotting, and radioimnmunoassay.

The invention includes an additional method of detecting the level of expression of hLhx3a in a biological sample. The method comprises contacting the sample, or a portion thereof, with an hLhx3a-binding molecule, which is preferably, a nucleic acid or an antibody. The hLhx3a-binding molecule specifically binds to hLhx3a present in the sample, and any hLhx3a-binding molecule not specifically bound to hLhx3a is removed from the mixture. The hLhx3a-binding molecule which remains bound to the hLhx3a is then detected by standard methods.

In one aspect, the hLhx3a-binding molecule is an antibody molecule which specifically binds to hLhx3a but which does not bind to hLhx3b. The antibody may be used to probe a Western blot of proteins present in a biological sample, such as serum or tissue, comprising hLhx3a. The Western blot is then washed to remove any antibodies which are not specifically bound to hLhx3a and the bound antibodies are detected by incubating the blot in a secondary antibody conjugated to a detector molecule. Further, the antibody can be used in an ELISA-based assay to quantitate the amount of hLhx3a present in a sample using standard methods well-known in the art.

In one embodiment, the hLhx3a-binding molecule is rabbit polyclonal anti-hLhx3a antibody and the secondary antibody is goat anti-rabbit conjugated to an enzyme such as horse radish peroxidase or alkaline phosphatase. Alternatively, the hLhx3a-binding molecule may be labeled directly such that the molecule may be detected directly.

In another aspect, the hLhx3a-binding molecule is a nucleic acid complementary to at least a portion of a nucleic acid encoding hLhx3a and sharing at least about 45% homology with hLhx3a (SEQ ID NO:23). Thus, the present invention includes detection of the level of expression of hLhx3a in a sample by detecting the presence of a nucleic acid encoding hLhx3a in the sample. For instance, RNA encoding hLhx3a may be detected by probing a Northern blot with a nucleic acid encoding the 5′ region of hLhx3a (SEQ ID NO:23). Any nucleic acid not specifically bound to the blot is removed by washing the filter and any nucleic acid specifically bound thereto is detected by a standard nucleic acid detection method such as those well known in the art. Stringency conditions that can be used to detect a nucleic acid specifically bound with the blot include, for example, washing the blot at about 50° C. using 2× SSC. However, the invention is not to be construed to be limited to any particular method of detecting the hLhx3a-binding molecule.

The amount of hLhx3a or nucleic acid encoding hLhx3a present in a sample is correlated to the level of expression of hLhx3 a in the sample. Therefore, by detecting the hLhx3a-binding molecule specifically bound with hLhx3a present in a sample, the level of expression of hLhx3a in the sample can be readily determined. One skilled in the art would appreciate, based upon the disclosure provided herein, that hLhx3a-binding molecules encompass antibodies that specifically bind with hLhx3a, or a portion thereof, or antibodies that bind with a tag polypeptide covalently linked with hLhx3a such that detecting the tag polypeptide portion of the protein also detects hLhx3a. Moreover, the hLhx3a-binding molecule also encompasses nucleic acids that specifically hybridize with a nucleic acid encoding hLhx3a such that the nucleic acid encoding hLhx3a can be detected using standard methods such as, but not limited to, Southern and Northern blotting, and the like as described in, e.g., Sambrook et al., supra, and Ausubel et al., supra. Methods of detecting the level of expression of a protein and/or nucleic acid encoding a protein are well-known in the art and include, but are not limited to, PCR, real-time PCR, Northern blotting, RNA dot blotting, RNase protection assay, and in situ hybridization, among others. Methods of detecting a protein include, but are not limited to, immunocytochemistry, ELISA, immunoradiometric assay, Western blotting, and radioimmunoassay.

The invention includes a method of identifying a compound which affects the activity of human Lhx3 in a cell. The method comprises contacting a cell with a compound and comparing the level of activity of Lhx3 in the cell with the level of activity of Lhx3 in an otherwise identical cell not contacted with the compound. A higher or lower level of activity of Lhx3 in the cell contacted with the compound compared with the level of activity of Lhx3 in the otherwise identical cell not contacted with the compound, indicates that the compound affects Lhx3 activity in a cell.

One skilled in the art would understand, based upon the disclosure provided herein, that Lhx3 activity encompasses, but is not limited to, the ability of hLhx3 to induce expression of a reporter gene operably linked to a αGSU promoter, the ability of hLhx3 to induce expression of a reporter gene operably linked to a TSHβpromoter, the ability of hLhx3 to bind with a nucleic acid that specifically binds with hLhx3, such as, but not limited to, the −350 to −323 bp region of murine αGSU promoter and/or the Lhx3 consensus binding sequence, activation of the gene encoding Pit-1, and activation of the gene encoding prolactin. The level of these various activities of hLhx3 can be assessed according to the assay methods described herein or by similar methods as would be apparent to one skilled in the art once armed with the disclosure provided herein.

The invention also includes a method of identifying a compound which affects the level of activity of Lhx3 in a cell where the cell has no detectable endogenous Lhx3 activity or where additional Lhx3 activity is provided to the cell by, among other things, transfecting the cell with vector encoding Lhx3 and a reporter gene construct where the reporter gene is operably linked to a promoter/regulatory sequence that is trans-activated by Lhx3. The co-transfected cell is then contacted with a test compound, and the level of Lhx3 activity in the cell is compared with the level of Lhx3 activity in an otherwise identical cell not contacted with the compound, or with the level of Lhx3 in the cell prior to being contacted with the compound. A higher or lower level of activity of Lhx3 in the cell contacted with the compound compared with the level of activity of Lhx3 in the cell prior being contacted with the compound or with the level in the otherwise identical cell not contacted with the compound, is an indication that the compound affects the level of activity of Lhx3 in a cell.

As disclosed previously elsewhere herein, the level of Lhx3 activity in the cell is assessed using the methods disclosed herein or methods as would be understood by one skilled in the art based upon the disclosure provided herein.

The invention also includes a method of identifying a compound which affects the level of activity of Lhx3a but not the level of activity of Lhx3b in a cell. The method comprises transfecting a cell having no detectable endogenous hLhx3 activity with an expression vector encoding Lhx3a and transfecting either the same cell, or an otherwise identical cell not transfected with a vector encoding hLhx3b. Each cell is also transfected with a reporter gene construct comprising a reporter gene operably linked to a promoter/regulatory sequence trans-activated by Lhx3a and Lhx3b (e.g., the murine PGBE sequence, the Lhx3 consensus binding sequence [gatcccagaaaattaattaattgtaa; SEQ ID NO:43], and the like). The cell is contacted with a test compound and the level of Lhx3a activity in each cell is assessed before and after contacting the cell with the test compound.

Similarly, the level of hLhx3b activity is assessed before and after contacting the cells with the compound. The level of activity of hLhx3a and hLhx3b before and after contacting each cell with the compound is compared. There is an indication that the test compound affects the level of hLhx3a activity but not the level of hLhx3b activity in a cell where there is (1) a higher or lower level of activity Lhx3a in the cell expressing hLhx3a which was contacted with the test compound compared with the level of activity of Lhx3a in the cell prior to or in the absence of contacting the cell with the test compound, and (2) there is no detectable change in the level of Lhx3b activity in the cell comprising a vector expressing hLhx3b which was contacted with the test compound compared with the level of Lhx3b activity in the cell prior to or in the absence of contacting the cell with the test compound. One skilled in the art would understand, based upon the disclosure provided herein, that there is no temporal requirement as to the order in which the level of hLhx3a and hLhx3b activity is determined. That is, the level of hLhx3a and b activity can be determined contemporaneously, or the level of either hLhx3 isoform can be determined followed by determination of the level of activity of the other isoform.

Thus, a compound that differentially affects the level of activity of one isoform of the protein (i.e. hLhx3a) but which does not affect the level of activity of another isoform of the protein (i.e., hLhx3b) is identified. Such a compound is useful in that it may be beneficial to increase or decrease the level of activity of one hLhx3 isoform where the level is higher or lower than the normal level but where the level of activity of the other isoform is not higher or lower than normal or where the level of the other isoform is at a desirable level. Therefore, the compound can be used to affect the level of one isoform while leaving the level of the other isoform unchanged where it is desirable or advantageous to affect the level of one isoform without affecting the other.

The invention includes a method of identifying a compound which affects binding of hLhx3 to a nucleic acid that specifically binds with Lhx3. The method comprises contacting an extract with (e.g., adding a compound to an extract) where the extract comprises hLhx3 and a nucleic acid that specifically binds with Lhx3. The level of binding of hLhx3 (i.e., hLhx3a, hLhx3b, or both) in the presence of the compound is compared with the level of binding in an otherwise identical extract to which the compound has not been added. A higher or lower level of binding of Lhx3 with the nucleic acid in the extract comprising the compound compared with the level of binding in the extract not comprising the compound indicates that the compound affects binding of Lhx3 with a nucleic acid that specifically binds with hLhx3. Thus, a compound that affects binding of hLhx3 with a nucleic acid that specifically binds with hLhx3 (e.g., a nucleic acid having nucleotides −350 to −323 of murine αGSU promoter, a nucleic acid encoding the Lhx3 consensus binding sequence [gatcccagaaaattaattaattgtaa; SEQ ID NO:43, the alpha-GSU sequence [gatccggtacttagctaattaaatga; SEQ ID NO:42], and the like), can be identified.

One skilled in the art would appreciate, based upon the disclosure provided herein, that hLhx3 mediates its function(s) by binding with a nucleic acid sequence thereby trans-activating a specific target gene (i.e., αGSU, TSHβ, and the like). Thus, a compound that affects the specific binding of hLhx3 with a nucleic acid that specifically binds with the protein is an important potential tool to affect the level of activity of hLhx3 in treating or alleviating a disease, disorder, or condition mediated by altered binding of hLhx3 to the nucleic acid.

The invention includes a method of identifying a compound for affecting Lhx3 induction of a pituitary trophic hormone gene promoter. The method comprises contacting a cell expressing hLhx3 (e.g., hLhx3a, hLhx3b, and the like) with a compound where the cell comprises a reporter gene construct comprising a thyroid-stimulating hormone beta promoter sequence operably linked to a reporter gene (e.g., green fluorescent protein, alkaline phosphatase, luciferase, beta-galactosidase, growth hormone, diphtheria toxin, chloramphenicol acetyl transferase, and the like). The cell further comprises a pituitary transcription factor that synergizes with hLhx3 (e.g., Pit-1, Pitx1/P-Otx, thyrotrope embryonic factor, and the like).

The level of expression of the reporter gene in the cell is assessed and compared with the level of expression of the reporter gene in an otherwise identical cell not contacted with the compound. A higher or lower level of reporter gene expression in a cell contacted with the compound compared with the level in an otherwise identical cell not contacted with the compound indicates that the compound affects hLhx3 induction of a pituitary trophic hormone gene promoter in the cell.

One skilled in the art would appreciate, based upon the disclosure provided herein, that the ability of hLhx3 to induce a pituitary trophic hormone gene promoter is an important function in the role of hLhx3 in pituitary development and function. Thus, a compound that affects the ability of hLhx3 to induce such a hormone and perform this function is an important potential therapeutic for treatment of a disease, disorder or condition mediated by the altered ability of hLhx3 to induce a pituitary trophic hormone. Further, such compound also provides an important assay for the study of the role of induction of a pituitary trophic hormone gene by hLhx3 in synergy with a pituitary transcription factor such as, but not limited to, Pit-1, in human disease and in the development and function of the pituitary in humans.

One skilled in the art would appreciate, based upon the disclosure provided herein, that a compound that increases the level of expression of Lhx3 compared with the level of Lhx3 expression in the untreated cell is potentially useful for the treatment of a disease, disorder or condition which is characterized by decreased levels of Lhx3 in afflicted individuals. Such disease, disorder or condition can included, but is not limited to, pituitary tumor formation, nervous system tumor formation, or other pituitary or nervous system disease, such as spina bifida, holoprosencephaly, mental retardation, combined pituitary hormone deficiency, pineal gland disorders, developmental delay, microcephaly, hypotonia, septo-optic dysplasia, and the like. Thus, the invention also includes a method of identifying a compound useful for the treatment of such disease, disorder or condition. Similarly, where a disease, disorder or condition is causally linked with increased level of Lhx3 in a cell compared to normal levels, a compound that decreases Lhx3 expression in a cell is useful to treat such disease, disorder or condition. Thus, identification of compounds that increase or decrease Lhx3 expression in a.cell are encompassed in the present invention.

The invention includes a method of assessing whether a test compound is useful for treatment of a disease, disorder or condition associated with altered binding of Lhx3 to a nucleic acid that specifically binds with hLhx3. In essence, the method identifies a test compound that increases binding of hLhx3 (e.g., hLhx3a, hLhx3b, and the like) with a nucleic acid that specifically binds with hLhx3 (e.g., the Lhx3 consensus binding sequence, the αGSU hormone promoter sequence, and the like).

One skilled in the art, based upon the disclosure provided herein, would appreciate that the binding assays disclosed herein can be used to compare the binding of hLhx3 with a nucleic acid as described in the various embodiments exemplified herein. Thus, the method comprises making a preparation comprising the test compound, a labeled nucleic acid that specifically binds with hLhx3, and at least a portion of hLhx3, under conditions where such binding would typically take place. Preferably, the portion of hLhx3 comprises a portion of hLhx3 that specifically binds with the nucleic acid that specifically binds hLhx3 used in the method.

One skilled in the art would also appreciate, based upon the disclosure provided herein, that the method of contacting hLhx3 and its target nucleic acid, whether hLhx3, the nucleic acid, or both, are labeled, the type of label, or how the bound/unbound nucleic acid and/or hLhx3 is detected are not crucial factors in the assay. That is, any number of labels (e.g., tag polypeptide epitope, radiolabels, and the like) can all be employed in the method. Moreover, either the nucleic acid can be labeled (as exemplified herein) or hLhx3 can be labeled. Further, the present invention is not limited to detecting hLhx3 binding with a nucleic acid using electrophoretic mobility shift assays (EMSA). Rather, the invention includes a variety of binding assays to detect protein binding with nucleic acid including, but not limited to, DNase footprint assays, Southwestern blotting, DNA affinity chromatography, screening of protein expression libraries using a nucleic acid probe, and the like.

As discussed previously elsewhere herein, a compound that affects binding of hLhx3 with a nucleic acid that specifically binds with hLhx3 is an important candidate therapeutic since the binding of hLhx3 to its associated nucleic acid has been demonstrated, by the data disclosed herein, to be associated with induction of specific pituitary hormone genes. Thus, the instant method provides an important assay in the development of useful compounds for the treatment of a disease, disorder, or condition mediated by altered binding of hLhx3 with a nucleic acid that specifically binds with hLhx3.

The invention further includes a method of identifying a human patient afflicted with a disease, disorder or condition associated with altered expression of Lhx3. The method comprises assessing the level of Lhx3 expression in a human suspected of being afflicted with a disease, disorder or condition associated with altered expression of Lhx3, and comparing the level of expression of Lhx3 with the level of expression of Lhx3 in a normal human not afflicted with a disease, disorder or condition associated with altered expression of Lhx3. Such comparison allows the identification of a human patient afflicted with a disease, disorder or condition associated with altered expression of Lhx3. Potential symptoms of a disease, disorder, or condition associated with altered expression of Lhx3 can include, but are not limited to, combined pituitary hormone deficiency, dwarfism, hypothyroidism, lack of puberty, sterility, developmental delay, nervous system disorders, pineal gland disorders, and the like.

As noted previously elsewhere herein, proper expression of Lhx3 may be required for proper development and functioning of the pituitary such that over and/or under-expression of Lhx3 is associated with a disease, disorder, or condition associated with such altered expression. Thus, by detecting altered expression of Lhx3 in a human, a human suffering from a disease, disorder, or condition can be detected and diagnosed.

The invention includes a method of identifying a human patient afflicted with a disease, disorder or condition associated with altered level of binding of Lhx3 to a nucleic acid that specifically binds with Lhx3. The method comprises assessing the level of Lhx3 binding with a nucleic acid that specifically binds with Lhx3 in a human suspected of being afflicted with a disease, disorder or condition associated with altered level of binding of Lhx3 to a nucleic acid that specifically binds with Lhx3, and comparing that level with the level of Lhx3 binding with a nucleic acid that specifically binds with Lhx3 in a normal human not afflicted with a disease, disorder or condition associated with altered level of Lhx3 binding with a nucleic acid that specifically binds with Lhx3. Thus, a higher or lower level of Lhx3 binding with a nucleic acid that specifically binds with Lhx3 in the human compared with a normal human that is not afflicted is an indication that the human is afflicted with a disease, disorder or condition associated with altered level of Lhx3 binding with a nucleic acid that specifically binds with Lhx3. In this way, a human patient afflicted with a disease, disorder or condition associated with altered level of Lhx3 binding with a nucleic acid that specifically binds with Lhx3 can be identified and diagnosed.

Based upon the disclosure provided herein, one skilled in the art would appreciate that a sample taken from a human potentially afflicted with a disease, disorder or condition mediated by altered binding of hLhx3 (e.g., hLhx3a, hLhx3b, or both) with a nucleic acid that specifically binds with hLhx3 (e.g., the Lhx3 consensus binding sequence, the αGSU hormone promoter sequence, and the like), can be assayed using the methods disclosed herein (e.g., electrophoretic mobility shift assays [EMSA]) to determine whether the human demonstrates altered hLhx3/nucleic acid binding. Such altered binding can be associated with a disease, disorder or condition since hLhx3 binding with a nucleic acid that specifically binds with hLhx3 is crucial to proper control of pituitary hormone gene expression mediated by such binding. Thus, a human suffering from a disease, disorder, or condition mediated by altered binding of hLhx3 with a nucleic acid that specifically binds with hLhx3 can be diagnosed and detected.

C. Methods of Detecting Mutations in the Lhx3 Gene Locus

The methods of the invention can be used to detect mutations in a nucleic acid of the invention in order to determine if a human has a mutated gene since such a human is potentially at risk for a disease, disorder, or condition associated with altered expression or activity of hLhx3. This is because it is well-known that most mutations are deleterious such that a human having a mutation in the nucleic acid encoding Lhx3 is more likely than not to be negatively impacted by such mutation which is potentially associated with altered expression or activity of the protein encoded by the mutated nucleic acid.

In certain embodiments, the methods include detecting, in a sample of cells obtained from the human, the presence or absence of a mutation characterized by at least one of an alteration of a nucleic acid encoding a hLhx3 of the invention, or the altered expression of the gene encoding hLhx3. For example, such mutations can be detected by ascertaining the existence of at least one of: 1) a deletion of one or more nucleotides from the nucleic acid encoding hLhx3; 2) an addition of one or more nucleotides to the nucleic acid encoding hLhx3; 3) a substitution of one or more nucleotides of the nucleic acid encoding hLhx3; 4) a chromosomal rearrangement of the nucleic acid encoding hLhx3; 5) an alteration in the level of a messenger RNA transcript of the nucleic acid encoding hLhx3; 6) an aberrant modification of the gene, such as of the methylation pattern of the genomic DNA; 7) a non-wild type splicing pattern of a messenger RNA transcript of the nucleic acid encoding hLhx3; 8) a non-wild type level of the protein encoded by the nucleic acid encoding hLhx3; 9) an allelic loss of the nucleic acid encoding hLhx3; and 10) an inappropriate post-translational modification of the protein encoded by the nucleic acid encoding hLhx3. As described herein, there are a large number of assay techniques known in the art which can be used for detecting such mutations in a nucleic acid encoding a known protein. Thus, once armed with the teachings set forth herein, including the nucleic and amino acid sequences of human and porcine Lhx3, and isoforms thereof, and the full length sequence of human Lhx3 disclosing the genomic arrangement of the entire sequence (SEQ ID NO:22), one skilled in the art would be able to detect a mutation in the LHX3 gene.

In certain embodiments, detection of the mutation involves the use of an primer in a polymerase chain reaction (PCR; see, e.g., U.S. Pat. Nos. 4,683,195 and 4,683,202), such as anchor PCR or RACE PCR, or, alternatively, in a ligation chain reaction (LCR; see, e.g., Landegran et al., 1988, Science 241:1077-1080; and Nakazawa et al., 1994, Proc. Natl. Acad. Sci. USA 91:360-364), the latter of which can be particularly useful for detecting point mutations in a gene (see, e.g., Abravaya et al., 1995, Nucleic Acids Res. 23:675-682). This method can include the steps of collecting a sample of cells from a patient, isolating nucleic acid (e.g., genomic, mRNA, or both) from the cells of the sample, contacting the nucleic acid sample with one or more primers which specifically hybridize with the selected gene under conditions such that hybridization and amplification of the gene (if present) occurs, and detecting the presence or absence of an amplification product. The method can also include detecting the size of the amplification product and comparing the length to the length of a corresponding product obtained in the same manner from a control sample. PCR, LCR, or both can be used as a preliminary amplification step in conjunction with any of the techniques used for detecting mutations described herein.

Alternative amplification methods include: self-sustained sequence replication (Guatelli et al., 1990, Proc. Natl. Acad. Sci. USA 87:1874-1878), transcriptional amplification system (Kwoh, et al., 1989, Proc. Natl. Acad. Sci. USA 86:1173-1177), Q-Beta Replicase (Lizardi et al., 1988, Bio/Technology 6:1197), or any other nucleic acid amplification method, followed by the detection of the amplified molecules using any of a variety of techniques well known to those of skill in the art. These detection schemes are especially useful for detection of nucleic acid molecules if such molecules are present in very low numbers.

In an alternative embodiment, mutations in a selected gene can be identified in a sample by detecting alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA (i.e., restriction fragment length polymorphism, RFLP) indicates occurrence of mutations or other sequence differences in the sample DNA compared with control, wild type DNA.

Moreover, sequence specific ribozymes (see, e.g., U.S. Pat. No. 5,498,531) can be used to detect the presence of specific mutations by development or loss of a ribozyme cleavage site.

In other embodiments, genetic mutations are identified by hybridizing a sample and control nucleic acids, e.g., DNA or RNA, with high density arrays containing hundreds or thousands of oligonucleotides probes (Cronin et al. (1996) Human Mutation 7:244-255; Kozal et al. (1996) Nature Medicine 2:753-759.

In addition, any of a variety of sequencing methods known in the art can be used to directly sequence the selected gene and detect mutations by comparing the sequence of the sample nucleic acids with the corresponding wild-type (control) sequence (see, e.g., Maxam and Gilbert, 1977, Proc. Natl. Acad. Sci. USA 74:560; Sanger, 1977, Proc. Natl. Acad. Sci. USA 74:5463. It is also contemplated that any of a variety of automated sequencing procedures can be used when performing the diagnostic assays (as reviewed in 1995, Bio/Techniques 19:448). Such automated sequencing methods include mass spectrometry (see, e.g., PCT Publication No. WO 94116101; Cohen et al., 1996, Adv. Chromatogr. 36:127-162; Griffin et al., 1993, Appl. Biochem. Biotechnol. 38:147-159).

Other methods for detecting mutations in a selected gene include methods involving protection from cleavage agents to detect mismatched bases in RNA/RNA or RNA/DNA heteroduplexes as described in, e.g., Myers et al. (1985, Science 230:1242). In essence, hybridizing RNA or DNA containing wild-type sequence with potentially mutant RNA or DNA obtained from a tissue sample and subsequent treatment of the duplexes formed with an agent(s) (e.g., S1 nuclease, hydroxylamine or osmium tetroxide with piperidine, DNA mismatch enzymes such as mutY from E. coli or mammalian thymidine DNA glycosylase) that cleaves single-stranded regions of duplex detects base pair mismatches between the control and sample strands. Following digestion of the mismatched regions, the resulting material is separated by size on denaturing polyacrylamide gels to determine the site of the mutated or mismatched region (see, e.g., Cotton et al., 1988, Proc. Natl. Acad. Sci. USA 85:4397; Saleeba et al., 1992, Methods Enzymol. 217:286-295).

In other embodiments, alterations in electrophoretic mobility are used to identify mutations in genes. For example, single strand conformation polymorphism (SSCP) analysis can be used to detect differences in electrophoretic mobility between mutant and wild type nucleic acids as described in Orita et al. (1989, Proc. Natl. Acad. Sci. USA 86:2766), Cotton (993, Mutat. Res. 285:125-144), and Hayashi (1992, Genet. Anal. Tech. Appl. 9:73-79).

In yet another embodiment, the movement of mutant or wild-type fragments in polyacrylamide gels containing a gradient of denaturant is assayed using denaturing gradient gel electrophoresis (DGGE), as described (Myers et al., 1985, Nature 313:495).

Examples of other techniques for detecting point mutations include, but are not limited to, selective oligonucleotide hybridization, selective amplification, and selective primer extension (see, e.g., Saiki et al., 1986, Nature 324:163; Saiki et al., 1989, Proc. Natl. Acad. Sci. USA 86:6230).

Alternatively, allele specific amplification technology can be used in conjunction with the methods of the invention as described in, for example, Gibbs et al. (1989, Nucleic Acids Res. 17:2437-2448), Prossner (1993, Tibtech 11:238), Gasparini et al. (1992, Mol. Cell Probes 6:1), and Barany (1991, Proc. Natl. Acad. Sci. USA 88:189).

Additional methods of detecting a mutation include fluorescent in situ hybridization (FISH), as disclosed elsewhere herein, and similar methods well-known in the art.

The methods described herein can be performed, for example, using pre-packaged diagnostic kits comprising at least one probe nucleic acid or antibody reagent described herein. Such kits can be used, for example, to diagnose a human patient exhibiting a disease, disorder, or condition involving a nucleic acid encoding hLhx3. Furthermore, any cell type or tissue in which the polypeptide of the invention is expressed, e.g., a blood sample containing peripheral blood leukocytes for proteins which are secreted or which occur on or in peripheral blood leukocytes, as well as cells and/or tissue from the pituitary, the brain, the pineal gland, the central nervous system, the spinal cord, motor neurons, and the like, can be used in the prognostic assays described herein.

VIII. Kits

The invention includes various kits which comprise a compound, such as an isolated nucleic acid encoding hLhx3b or hLhx3a in a sense or in an antisense orientation, or an isolated Lhx3 polypeptide, or the antibodies of the invention, and instructional materials which describe use of the compound to perform the methods of the invention. Although exemplary kits are described below, the contents of other useful kits will be apparent to the skilled artisan in light of the present disclosure. Each of these kits is included within the invention.

The invention includes a kit for determining the absence or presence of a nucleic acid encoding hLhx3 (e.g., hLhx3a, hLhx3b, or both) in a cell and/or in a sample. The kit comprises a primer pair which amplifies a unique target sequence of the human genome encoding hLhx3a, hLhx3b, or both. In a preferred embodiment, the target hLhx3 nucleic acid is the sequence encoding the amino terminal portion of hLhx3a (from about nucleotide −104 to about nucleotide 78 of SEQ ID NO:9) (i.e., SEQ ID NO:23) and the forward and reverse primers have the sequences SEQ ID NO:34 and SEQ ID NO:35, respectively. Further, the internal oligonucleotide probe used to detect the amplified nucleic acid has the sequence of SEQ ID NO:38.

In another preferred embodiment, the target hLhx3 nucleic acid is the sequence encoding the amino terminal portion of hLhx3b (from about nucleotide −119 to about nucleotide 93 of SEQ ID NO:11) (i.e., SEQ ID NO:24), and the forward and reverse primers have the sequences SEQ ID NO:36 and SEQ ID NO:37, respectively. Further, the internal oligonucleotide probe used to detect the amplified nucleic acid has the sequence of SEQ ID NO:39.

One skilled in the art would understand, based upon the disclosure provided herein, that the present invention is not limited to these primers or oligonucleotide probe sequences. Rather, any oligonucleotide probe having a sequence between the two primers spanning from nucleotide residue −104 up to and including nucleotide residue 78 of a nucleic acid having the sequence SEQ ID NO:9 (hLhx3a), and from nucleotide residue −119 up to and including nucleotide residue 93 of a nucleic acid having the sequence SEQ ID NO:11 (hLhx3b), is encompassed in the invention.

The kit is used pursuant to the methods disclosed in the invention. Briefly, the kit can be used in standard PCR, or in a homogeneous format PCR procedure (e.g., QSD) as described herein. One skilled in the art would appreciate based on the disclosure herein that the kit may used in any of the afore-mentioned procedures and in any combination thereof.

In one aspect, the invention includes a kit for affecting the level of expression of hLhx3a in a cell. The kit is used pursuant to the methods disclosed in the invention. Briefly, the kit may be used to introduce an isolated nucleic acid encoding hLhx3b or hLhx3a into a cell in order to increase the level of expression of Lhx3 in the cell. Moreover, the kit comprises an instructional material for the use of the kit. These instructions simply embody the disclosure provided herein.

The invention also includes a kit for inhibiting the level of expression of hLhx3a in a cell. The kit may be used to introduce an isolated nucleic acid encoding hLhx3b or hLhx3a in an antisense orientation thereby reducing the level of expression of Lhx3 in the cell. The kit further comprises an applicator useful for introducing the antisense nucleic acid into the cell. The particular applicator included in the kit will depend on the recombinant DNA method used to introduce the nucleic acid into the cell and such applicators are well-known in the art and may include, among other things, a pipette, a syringe, a dropper, and the like. Moreover, the kit comprises an instructional material for the use of the kit. These instructions simply embody the disclosure provided herein.

The invention further includes a kit for detecting the level of hLhx3b in a biological sample. The kit comprises an hLhx3b-binding molecule which specifically detects hLhx3b, an applicator therefor, and an instructional material for the use of the kit. The instructions simply embody the examples provided herein. For example, in one aspect, the hLhx3b-binding molecule is an anti-hLhx3b antibody which may be used to detect hLhx3b on Western blots or the antibody may be used to detect hLhx3b by enzyme-linked immunosorbent assay (ELISA), and the like.

In another aspect, the hLhx3b-binding molecule can be an isolated nucleic acid which specifically hybridizes to a nucleic acid encoding hLhx3b but not to one encoding hLhx3a. Such detection is exemplified herein using PCR primer pairs specific for hLhx3b. The amplification products can then be detected using a variety of methods including visualization of the amplicons on EtBr-stained gels, Southern blotting, and QSD using a fluorogenic internal oligonucleotide probe. Thus, the hLhx3b-binding molecule may be detected using immunological and/or recombinant DNA techniques well-known in the art and as described herein or in standard texts such as, for example, Sambrook et al., supra, and Ausubel et al., supra.

Since the level of hLhx3 (i e., hLhx3a, hLhx3b, or both) and/or the level of nucleic acid encoding hLhx3 present in a sample is correlated to the level of expression of hLhx3 in that sample, the level of expression of hLhx3 may be detected by detecting the level of hLhx3 and/or the level of nucleic acid encoding hLhx3. Thus, the kit is useful for detecting the level of expression of hLhx3 in a biological sample.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

### Characterization of the Porcine Lhx3/LIM-3 μP-Lim LIM Homeodomain Transcription Factor

Lhx3/LIM-3/P-Lim is a LIM homeodomain transcription factor that in mice is essential for development of the anterior and intermediate lobes of the pituitary gland. The data disclosed herein demonstrate the cloning and characterization of porcine Lhx3. The porcine Lhx3 protein exhibits strong similarity to murine Lhx3 within the amino terminal LIM domains and the homeodomain, but is divergent from the mouse sequence in regions outside these motifs.

Expression vectors for porcine Lhx3 activated murine and porcine alpha-glycoprotein subunit (αGSU) reporter genes in transfection assays, and recombinant porcine Lhx3 protein specifically bound to a target site within the porcine αGSU gene upstream sequence. In addition, porcine Lhx3 synergistically induced transcription from prolactin enhancer/promoter reporter genes in cooperation with the Pit-1 pituitary transcription factor. Porcine Lhx3 protein interacted with Pit-1 protein in solution and also with the LIM domain-binding protein NLI/Lbd1/CLIM.

Together, these data indicate that many aspects of Lhx3 function in the mammalian pituitary are conserved and that Lhx3 may be involved in the activation of trophic hormone genes during early and late stages of pituitary organogenesis. Divergence in the Lhx3 amino acid sequence between mammalian species may suggest distinct activities for this protein in some species and may help identify important functional domains of this key developmental transcription factor.

The data disclosed herein demonstrate cloning and characterization of porcine Lhx3 (pLhx3) and disclose conservation of Lhx3 structure and function. The data disclosed demonstrate that the two LIM domains and the homeodomain of the molecule are well conserved across species. The data demonstrate, for the first time, that other regions of the Lhx3 nucleic acid and protein encoded thereby are diverged. The pLhx3 gene produces a rare messenger RNA transcript in the adult pituitary gland. Porcine Lhx3 expression vectors can alone activate the αGSU gene promoter and can synergistically enhance transcription from the prolactin promoter in cooperation with Pit-1. Recombinant pLhx3 protein specifically binds to a recognition site in the porcine αGSU gene promoter and can interact with both Pit-1 and NLI proteins.

The Materials and Methods used in this Example are now described.

DNA Cloning/DNA Sequencing/Plasmid Construction

Eight million plaques of λgt11 and λgt22 porcine pituitary complementary DNA (cDNA) libraries were screened using the mouse Lhx3 protein coding region (Bach et al., 1995, Proc. Natl. Acad. Sci. USA 92:2720-2724) as a probe at high stringency (final wash=0.5× SSC, 65° C.). Complementary DNA was extracted from 6 purified, positive bacteriophage and inserts were cloned into pBluescript KS II-(Stratagene, La Jolla, Calif.) as described (Rhodes et al., 1996, J. Animal Sci. 74:94-106). Three clones were completely sequenced on both strands according to the Sanger dideoxy sequencing method using Sequenase (Amersham/USB, Amersham Pharmacia Biotech, Piscataway, N.J.). Sequence analysis and alignment were performed using Wisconsin Genetics/GCG and DNASIS (Hitachi, San Jose, Calif.) software. Two cDNA clones, e.g., 0.2 kb and 0.9 kb, were digested at a common Pst I restriction site and ligated together to form a 1.6 kb pLhx3 cDNA which was cloned into the Eco RI site of pBluescript KS II-.

Expression vectors for pLhx3, ΔLIM pLhx3 (amino acids 134 to 384) and myc epitope-tagged pLhx3 were generated using polymerase chain reaction (PCR) using the 1.6 kb pLhx3 cDNA as a template, Expand HiFi polymerase (Boehringer Mannheim, Indianapolis, Ind.) and the following primers:

5′-cgcaagcttccaccatgtgggaggggcggccacaggagctg-3′ (5′, pLhx3) [SEQ ID NO:54];
5′-cgggatccaagcagcgagaggccgaggccacgg-3′ (5′, ΔLIM pLhx3) [SEQ ID NO:55];
5′-cgcaagcttccaccatggagcagaag-ctgatcagcgaggaggacctgtgggaggggcggccacagga
gctgggag-3′ (5′, myc-pLhx3). [SEQ ID NO:56]
A T3 primer (5′-caattaaccctcactaaaggg-3′) [SEQ ID NO:57]

was used as the antisense primer and products were cloned into the Hind III/Eco RI sites of pcDNA3 (Invitrogen, Carlsbad, Calif.). These oligonucleotides introduce a synthetic Kozak initiator methionine (pLhx3, ΔLIM pLhx3), or a methionine plus an 11 amino acid myc epitope tag (myc-pLhx3). The sequences of all constructs were confirmed by sequencing.

A 1.1 kb fragment representing the −1059 to +36 basepairs (bp) region of the porcine alpha-glycoprotein (αGSU) gene promoter (Kato et al., 1990, J. Mol. Endo. 7:27-34) was cloned using PCR using porcine genomic DNA as template and the following primers: 5′-cggaattcatgaataatgatgatactaattc-3′ sense [SEQ ID NO:58], 5′-ccgctcgagggatattagcttgtcttgcgatttc-3′ antisense [SEQ ID NO:59]. This PCR fragment was cloned into pBluescript KS II- for sequencing. A G/C polymorphism at −997 bp was observed in multiple independent clones.

A porcine αGSU luciferase reporter gene was constructed by cloning the fragment into the Sma I/Xho I sites of pGL-3 Basic (Promega Corp., Madison, Wis.). The murine αGSU promoter luciferase plasmid was described previously by Roberson et al. (1994, Mol. Cell Biol. 14:2985-2993). The rat prolactin enhancer/promoter-luciferase plasmid was as described by Bach et al. (1995, Proc. Natl. Acad. Sci. USA 92:2720-2724).

Northern Analysis

Tissues were dissected from embryonic or adult pregnant female mixed strain pigs and immediately frozen in liquid nitrogen. The samples were ground to a powder on dry ice/liquid nitrogen and total RNA was extracted using Tri-Reagent (Molecular Research Center, Inc., Cincinnati, Ohio) and bromochloropropane as a chloroform substitute. RNA molecules were separated using denaturing, formaldehyde agarose gels followed by transfer to Nytran Plus membranes (Schleicher and Schuell, Inc., Keene, N.H.) using the Turboblotter system (Schleicher and Schuell).

Membranes were hybridized to radiolabeled cDNA probes in ExpressHyb buffer (ClonTech Inc. Palo Alto, Calif.) at 1×10⁶ cpm/ml. Probes were the 1.6 kb pLhx3 cDNA, a 0.4 kb fragment of porcine pre-proprolactin cDNA, a 1.2 kb porcine growth hormone cDNA and a 0.4 kb fragment of the porcine Pit-1 coding region. Fragments were labeled by random priming using Klenow fragment ³²P-dCTP (Amersham, Arlington Heights, Ill.) to a specific activity of >1×10⁹ cpm/μg. After hybridization, membranes were washed in 0.2× SSC at 60° C. followed by exposure to Kodak MR film (Eastman Kodak Co., Rochester, N.Y.) using intensifying screens.

In vitro Cell Culture, Transfection and Luciferase Assays

Human embryonic kidney 293 and 293T cells were cultured in Dulbecco's modified Eagle medium (DMEM, Gibco/BRL, Gaithersburg, Md.) with 10% fetal bovine serum (Irvine Scientific, Santa Ana, Calif.), 100 units/ml penicillin, 100 μg/ml streptomycin (Irvine Scientific, Santa Ana, Calif.). About 1.5×10⁵ cells/60 mm dish were transfected using calcium phosphate/DNA precipitates using the CalPhos system (ClonTech Inc. Palo Alto, Calif.). Approximately 2 μg of reporter plasmid and 1 μg of expression vector were added per 60 mm dish and all groups received equal final DNA concentrations (by balancing with appropriate vector plasmids). Control cultures received empty expression vector DNA. The cells were harvested for luciferase assay 48 hours following transfection. After rinsing with cold phosphate buffered saline (PBS), the cultures were lysed directly on the plates in 25 mM Tris (pH 7.8), 2 mM dithiothreitol, 1% Triton X-100, 2 mM ethylenediaminetetraacetic acid (EDTA), and 10% glycerol. Lysate supernatants were assayed for luciferase activity using a Berthold LB 9507 luminometer and luciferin substrate (Promega Corp., Madison, Wis.). All assays were performed in triplicate. Total cell protein was determined by the Bradford method (Bio-Rad Laboratories, Richmond, Calif.) and luciferase activity was normalized to protein concentration.

Western Blot Analysis

293 or 293T cells were transfected with myc-pLhx3 or control expression vectors as described previously elsewhere herein. Following transfection, the cells were rinsed with cold PBS and total protein was extracted using sonication in SDS protein loading dye. Proteins were separated using 12% SDS polyacrylamide gel electrophoresis (PAGE) and the separated proteins were transferred to polyvinyldifluoride membranes (Millipore, Bedford, Mass.). Membranes were stained with Coomassie Brilliant Blue to confirm the transfer of equal amounts of protein in both control and experimental lanes.

Membranes were washed in 0.01 M Tris (pH 7.5), 0.1 M NaCl, and 0.1% Tween 20, followed by blocking in the same buffer plus 1% bovine serum albumin. The blots were incubated with a mouse anti-myc monoclonal antibody (i.e., 9E10) ascites at 1:5000. After washing the blots, a secondary antibody (goat anti-mouse conjugated with horseradish peroxidase obtained from Cappel (ICN, Costa Mesa, Calif.) was used at a dilution of 1:40,000. The results were visualized using Renaissance chemiluminescence reagents (NEN® Life Science Products, Boston, Mass.) and Kodak MR film (Eastman Kodak Co., Rochester, N.Y.).

Recombinant Protein Preparation/Electrophoretic Mobility Shift Analysis

Bacterial expression vectors for glutathione-S-transferase (GST)-pLhx3 and GST-ΔLIM pLhx3 (amino acids 134 to 384) fusion proteins were generated by cloning Barn HI/Eco RI compatible fragments of the pLhx3 cDNA into pGEX-KT (Hakes and Dixon, 1992, Anal. Biochem. 202:293-298). Porcine Lhx3 cDNA fragments were generated by PCR using the following oligonucleotides:

| | |
|---|---|
| 5′-cgggatcctgggaggggcggccacaggagctg-3′ | [SEQ ID NO:60], and |
| 5′-cggaattcagtcagaactgagcgtgatcc-3′ | [SEQ ID NO:61] to generate pLhx3, and |
| 5′-cgggatccaagcagcgagaggccgaggccacgg-3′ | [SEQ ID NO:62] and |
| 5′-cggaattcagtcagaactgagcgtgatcc-3′ | [SEQ ID NO:63] to produce ΔLIM pLhx3. |

The sequences of the constructs were confirmed by sequencing.

Bacterial expression vectors were transformed into E. coli BL21 (DE3) pLysS. Transformants were grown at 37° C. in Luria broth, 100 μg/ml ampicillin, 25 μg/ml chloramphenicol to an optical density of 0.8 at 600 nm. Protein expression was then induced with 0.1 M isopropylthiogalactoside for 3 hours at 37° C. Cells were resuspended in 150 mM NaCl, 16 mM Na₂HPO₄, 4 mM NaH₂PO₄, 1 mM EDTA, 0.1% beta-mercaptoethanol (BME), 1 μg/ml leupeptin, 1 μg/ml pepstatin, and 0.2 M phenymethylsulfonyl fluoride.

The cells were lysed by sonication, and a slurry of glutathione agarose beads (Sigma Chemical Company, St. Louis, Mo.) with the same protease inhibitors was added to the supernatant for 2 hours at 4° C. After washing, fusion proteins were directly eluted in 0.05 M Tris (pH 8.0), 0.01 μM reduced glutathione, 0.1% BME. Alternatively, ΔLIM pLhx3 proteins were released by thrombin cleavage in 50 mM Tris (pH 8.0), 150 mM NaCl, 2.5 mM CaCl₂, 0.1% BME with 1.5 units of bovine serum thrombin (Sigma Chemical Company, St. Louis, Mo.), for 3 hours. The proteins were analyzed using 12% SDS-PAGE.

Electrophoretic mobility shift assays were performed as described (Rhodes et al., 1993, Genes Dev. 7:913-932; Rhodes et al., 1996, Mol. Cell Endocrinol. 124:163-172). Wild type and mutant oligonucleotides representing the −351 to −324 bp region of the porcine αGSU promoter were:

| | |
|---|---|
| 5′-acattaggtacttagctaattaaatgtg-3′ | (SEQ ID NO:64), |
| 5′-cacatttaattagctaagtacctaatgt-3′ | (SEQ ID NO:65), and |
| 5′-acattaggtacttggcgcgccaaatgtg-3′ | (SEQ ID NO:66), |
| 5′-cacatttggcgcgccaagtacctaatgt-3′ | (SEQ ID NO:67), |

respectively. In competition experiments 200 ng of unlabeled, competitor oligonucleotide was added to binding reactions.

Protein/Protein Interaction Assays

NLI (nuclear LIM interactor) was cloned using PCR methods from mouse skeletal muscle cDNA using the following primers:

| | |
|---|---|
| 5′-cgggatccatgctggatcgggatgtgggcccaac-3′ | [SEQ ID NO:68], |
| 5′-cggaattccgtcttctgctccctggagctgtg-3′ | [SEQ ID NO:69]. |

After sequencing, the NLI cDNA was introduced into pcDNA3 plasmid vector (Invitrogen, Carlsbad, Calif.). The rat Pit-1 cDNA plasmid (pMet Pit-1) was as described previously (Ingraham et al., 1988, Cell 55:519-529).

Labeled pLhx3, NLI and Pit-1 proteins were synthesized in vitro using TNT rabbit reticulocyte lysate reagents (Promega Corp., Madison, Wis.) and ³⁵S-methionine. Protein/protein interaction assays were performed essentially as described (Bach et al., 1995, Proc. Natl. Acad. Sci. USA 92:2720-2724). All buffers contained 100 μg/ml ethidium bromide (to prevent DNA-mediated, non-specific interactions) and 1 mM ZnCl₂ to stabilize LIM domains. Control reactions contained at least five-fold excess of GST protein resin. After binding, affinity resins were extensively washed and the bound proteins analyzed using SDS-PAGE followed by treatment with Amplify fluorography reagent (Amersham, Arlington Heights, Ill.) and exposure to Kodak MR film (Eastman Kodak Co., Rochester, N.Y.) at −80° C.

The Results of the experiments presented in this Example are now described.

Cloning of Porcine Lhx3 Complementary DNAs

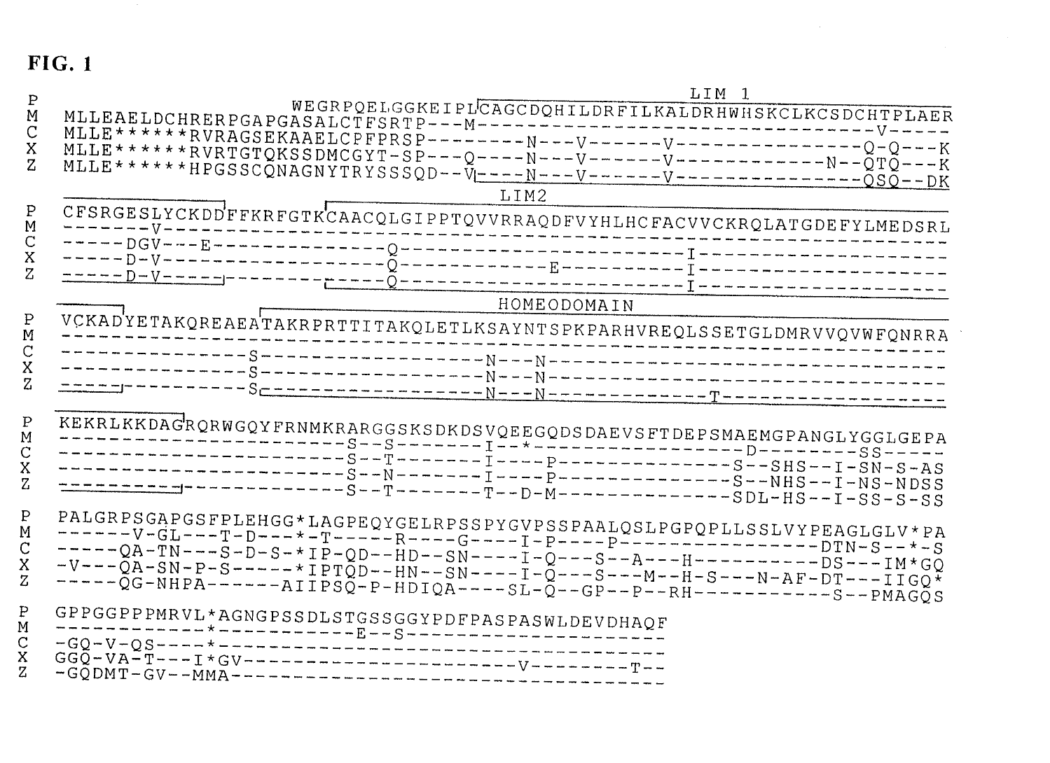

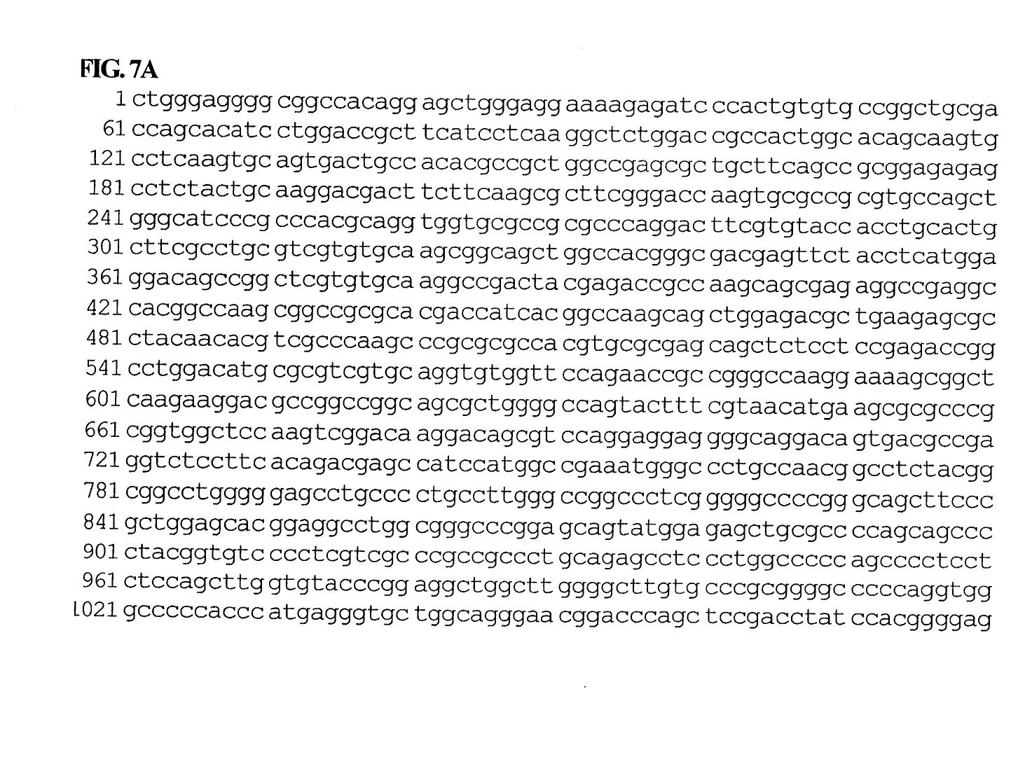

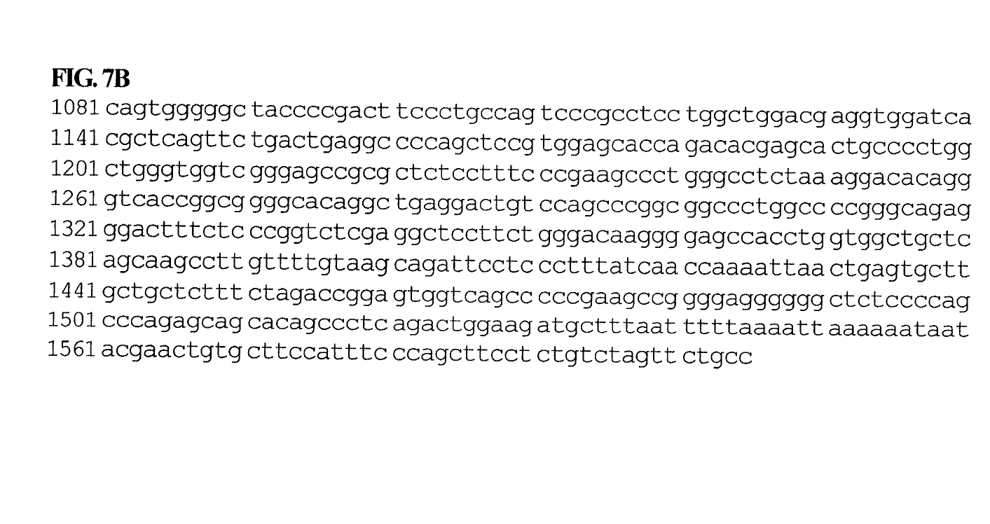

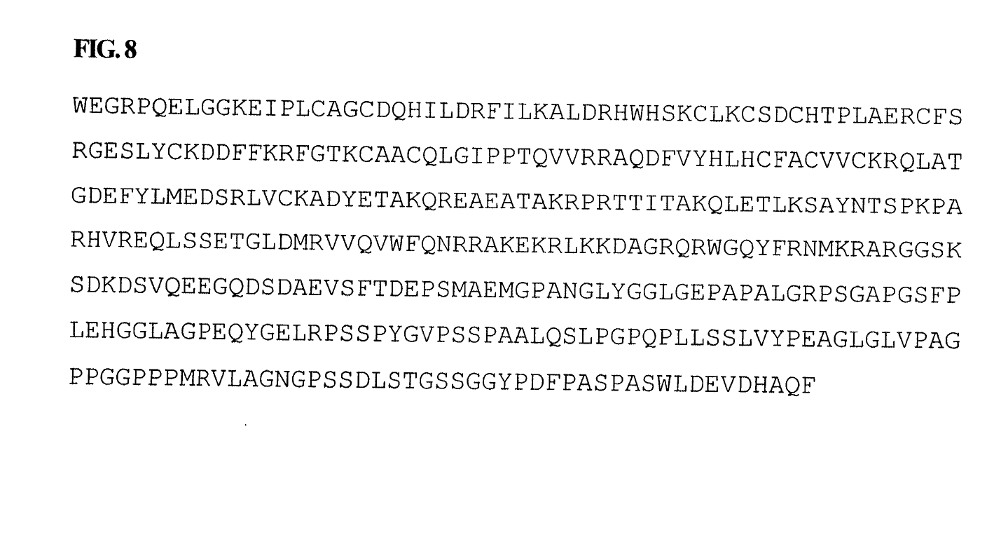

To identify cDNAs encoding porcine Lhx3, pituitary cDNA libraries were screened using the coding region of murine Lhx3 (Bach et al., 1995, Proc. Natl. Acad. Sci. USA 92:2720-2724) as a probe under conditions of moderate stringency. Bacteriophage containing six porcine Lhx3 cDNA clones were purified and the inserts were cloned into phagemids and sequenced. The deduced amino acid sequence of porcine Lhx3 (SEQ ID NO:2) was compared to that of murine Lhx3 and to related proteins in non-mammalian species (FIG. 1). The amino acid sequence of porcine Lhx3 is also shown in FIG. 8, and the nucleic acid sequence (SEQ ID NO:1) (GenBank accession number AF063245) is shown in FIG. 7.

Overall, the data disclosed herein demonstrate that porcine and murine Lhx3 proteins exhibit approximately 89% amino acid identity. The two amino-terminal LIM domains of Lhx3 are strongly conserved in the mammalian species, with complete identity between mouse and pig in the LIM 2 domain. In addition, the homeodomain is entirely conserved in these mammals and strongly maintained across evolution. Outside the LIM and DNA-binding domains, the Lhx3 proteins are less similar, except that the carboxyl terminal region is rich in serine and threonine residues. The final 34 amino acids are well conserved (as noted by Glasgow et al., 1997, Dev. Biol. 192:405-419), implying a possible important function for this sequence.

The amino terminal sequences of all Lhx3 family proteins diverge significantly. In the pig, this sequence is notably different from the mouse and other species (FIG. 1). However, the cDNAs encoding the initiator methionine of porcine Lhx3 had not been obtained despite multiple attempts using several methodologies, perhaps due to the high GC content of the DNA sequence. The studies disclosed below were therefore performed with plasmids derived from the longest cDNA identified.

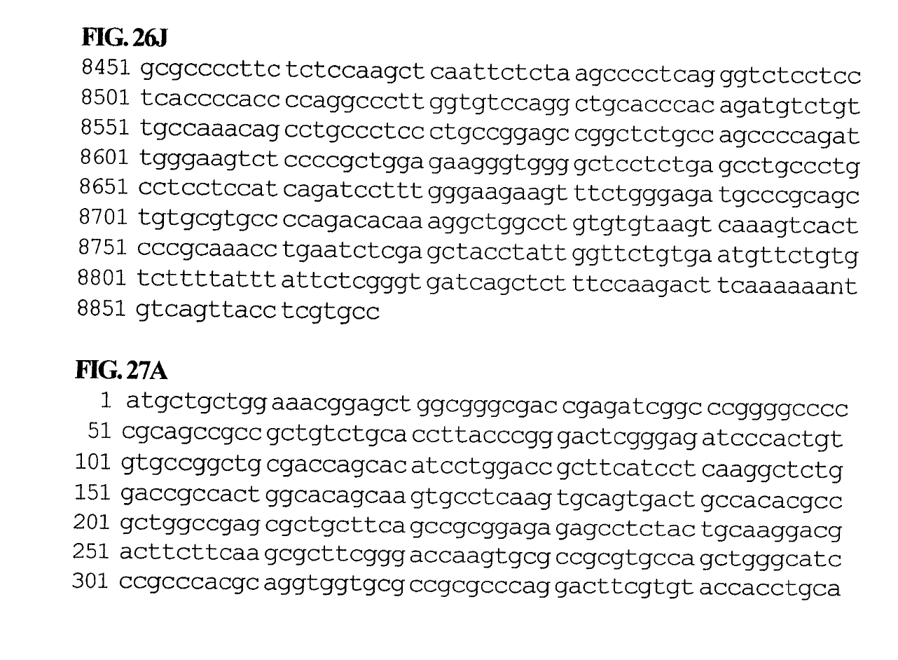

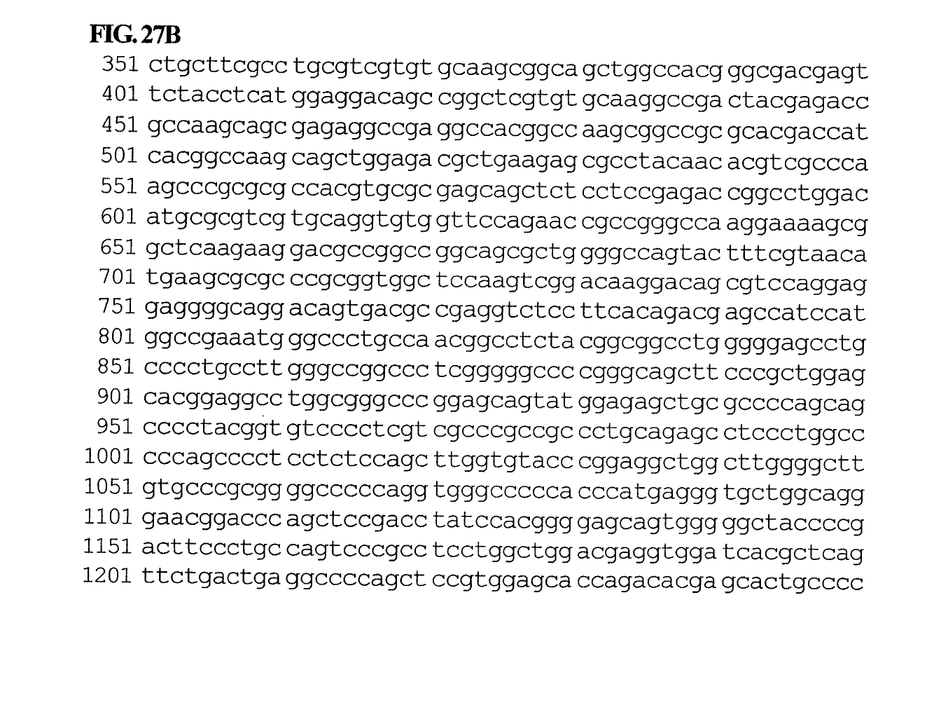

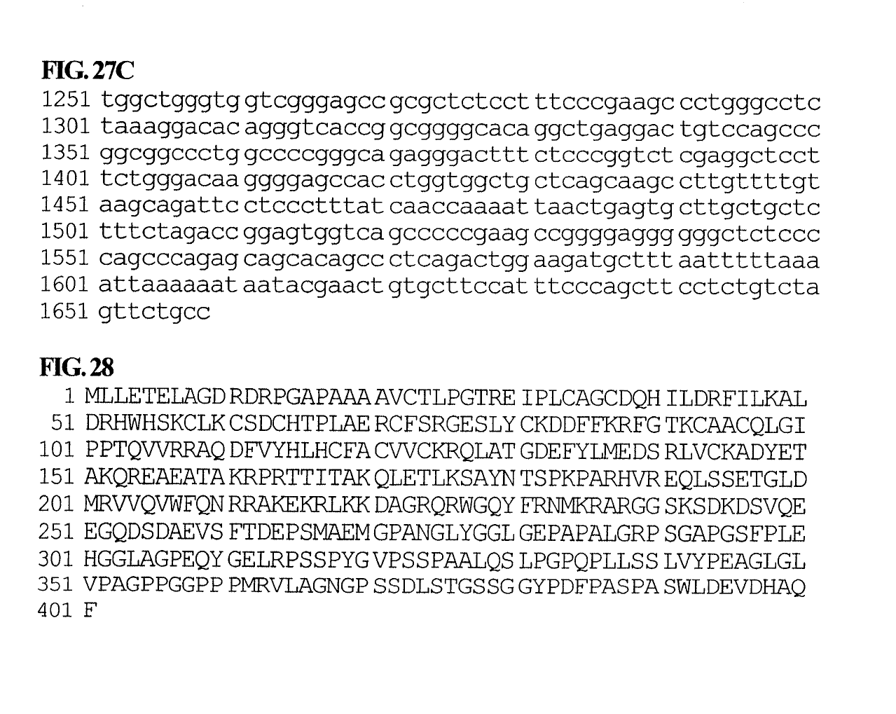

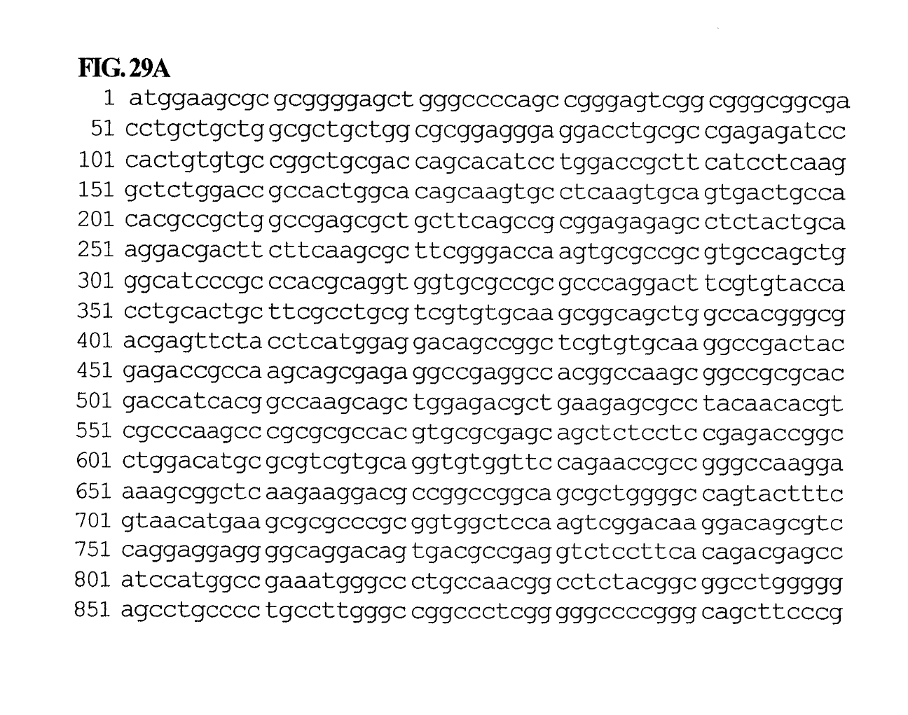

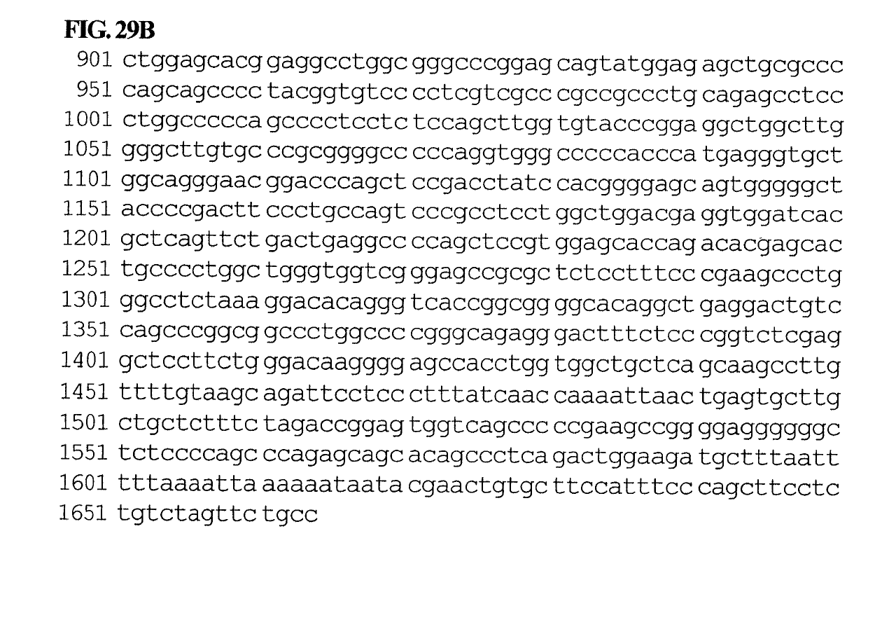

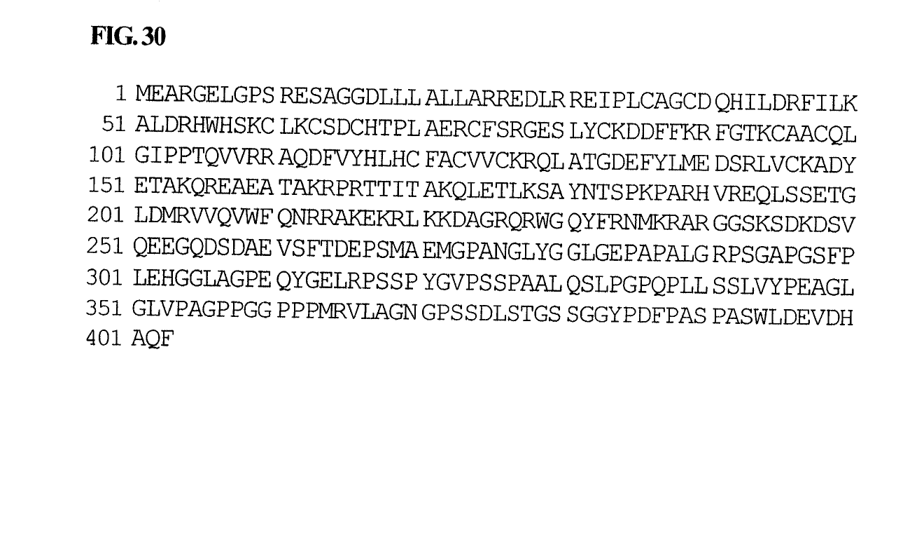

More recent data disclosed herein demonstrate the isolation and characterization of the full length coding sequence of porcine Lhx3. These data demonstrate, for the first time, that porcine, like human Lhx3, is expressed in at least two isoforms, termed Lhx3a and Lhx3b, which is also similar to the expression of murine Lhx3, which also comprises two isoforms termed mLhx3a and mLhx3b. The data disclosed herein demonstrate the nucleic acid sequence of pLhx3a (SEQ ID NO:13) (FIG. 27) and the amino acid sequence of pLhx3a (SEQ ID NO:14) (FIG. 28). The data disclosed herein also disclose the nucleic acid sequence of porcine Lhx3a (SEQ ID NO:15) (FIG. 29) and the amino acid sequence of pLhx3b (SEQ ID NO:16) (FIG. 30).

Analysis of Porcine Lhx3 Messenger RNA

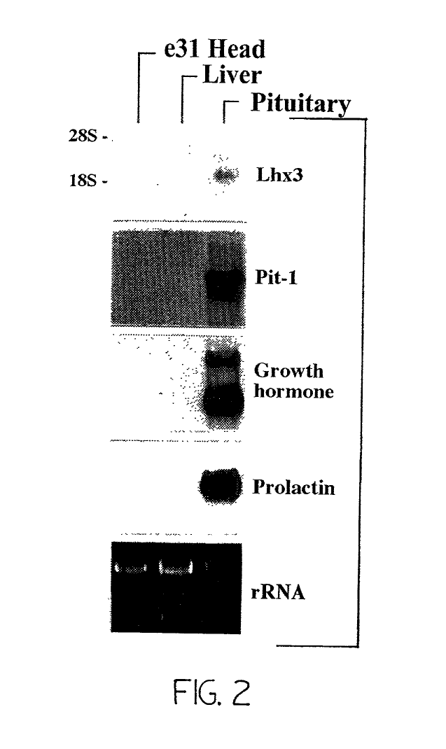

To confirm the expression of Lhx3 in the porcine pituitary, RNA was extracted from frozen tissues and subjected to Northern analysis (FIG. 2). Porcine Lhx3 cDNA probes hybridized to a rare mRNA of approximately 2.2 kb. In contrast, Pit-1 mRNAs were found to be expressed at high levels in the adult porcine pituitary (FIG. 2). The porcine Pit-1 gene produces two predominant mRNA species similar in size (approximately 2.1 and 1.5 kb) to those detected in the mouse, in contrast to the four distinct RNA species found in the rat anterior pituitary (Ingraham et al., 1988, Cell 55:519-529). Control experiments confirmed the abundant expression of porcine growth hormone and prolactin mRNAs of approximately 800 bp in the pituitary RNA samples (FIG. 2). Porcine Lhx3 mRNAs was not detected in total RNA samples from embryonic day 31 head tissue. Without wishing to be bound by any particular theory, this is likely due to low representation of Lhx3 mRNAs in these complex preparations.

The porcine growth hormone and prolactin genes are activated at later times of development than Lhx3 (Ma et al., 1996, Exp. Clin. Endocrinol. Diabetes 104:464-472; Granz et al., 1997, J. Neuroendo. 9:439-449) and were therefore not detected in the embryonic RNA sample.

Activation of Pituitary Hormone Genes by Porcine Lhx3

To examine the ability of porcine Lhx3 to activate pituitary genes, human embryonic kidney 293 cells were co-transfected heterologous with alpha-glycoprotein (αGSU) or prolactin luciferase reporter genes and CMV-promoter vectors expressing pLhx3, myc epitope-tagged pLhx3, or pLhx3 lacking both LIM domains (ΔLIM pLhx3). In order to allow investigation of regulation of the porcine αGSU promoter by pLhx3, the −1059 to +36 bp region of the gene (Kato et al., 1990, J. Mol. Endo. 7:27-34) was cloned from genomic DNA into a luciferase vector to create a novel reporter gene.

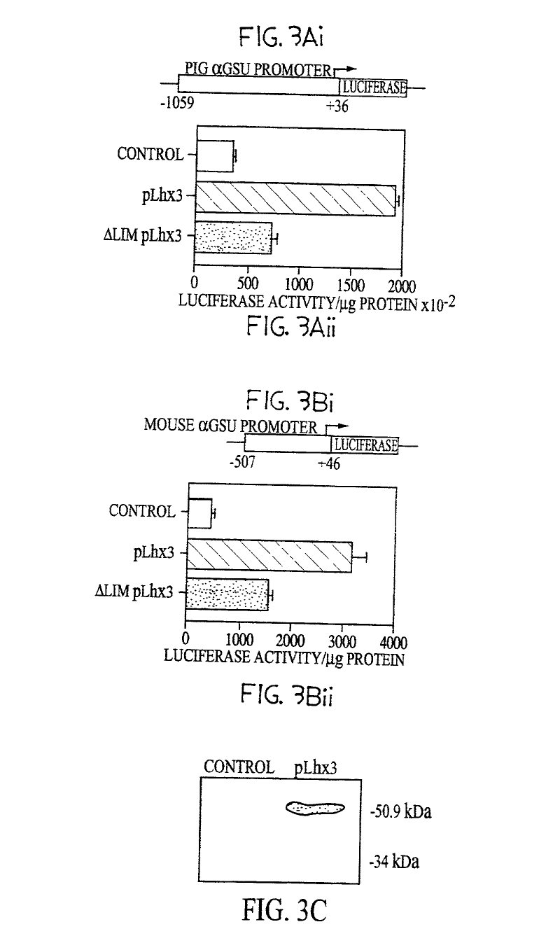

In these above-disclosed assays, pLhx3 activated the porcine and murine αGSU promoters four- to ten-fold (a typical experiment is disclosed in FIG. 3). In contrast to studies using murine Lhx3 (Bach et al., 1995, Proc. Natl. Acad. Sci. USA 92:2720-2724; Bach et al., 1997, Genes Dev. 11:1370-1380), deletion of the LIM domains impaired the trans-activation ability of porcine Lhx3, although the truncated protein could still significantly activate αGSU promoter reporter genes (FIGS. 3A and 3B). Using Western analysis, a single protein species of approximately 50 kDa was detected in 293 cells transfected with myc epitope-tagged pLhx3 (FIG. 3C). This apparent molecular weight is slightly larger than that predicted from the myc-pLhx3 open reading frame and may indicate modification of the protein in these cells.

Murine Lhx3 and LH-2/Lhx2 LIM homeodomain factors have been shown to recognize the pituitary glycoprotein hormone basal element (PGBE) located 323 basepairs upstream of the murine αGSU promoter (Roberson et al., 1994; Mol. Cell Biol. 14:2985-2993; Bach et al., 1995, Proc. Natl. Acad. Sci. USA 92:2720-2724; Bach et al., 1997, Genes Dev. 11:1370-1380). The data disclosed herein demonstrate that a motif located at −351 to −324 bp of the porcine αGSU promoter shows identity to the murine PGBE sequence within the functional core defined by Roberson et al. (1994, Mol. Cell Biol. 14:2985-2993), but differs outside of the core. Electrophoretic mobility shift assays (EMSA) were used to demonstrate that porcine Lhx3 specifically binds to this porcine motif sequence in the αGSU promoter.

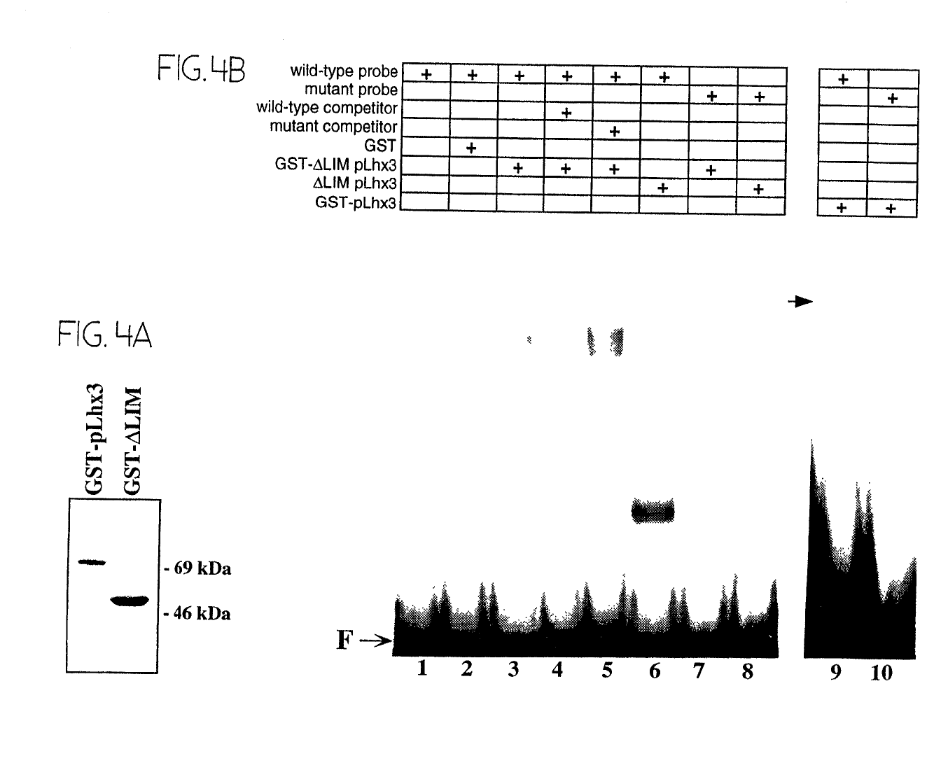

Recombinant glutathione-S-transferase (GST) fusion proteins containing pLhx3 (GST-pLhx3) and pLhx3 lacking both LIM domains (GST-ΔLIM pLhx3) were expressed in E. coli using the pGEX-KT vector (Hakes and Dixon, 1992, Anal. Biochem. 202:293-298) and purified by affinity chromatography. Recovered proteins were detected as single protein species of the predicted molecular weights on SDS polyacrylamide gels (FIG. 4A).

GST-ΔLIM pLhx3 strongly bound a labeled probe representing the porcine αGSU −351 to −324 bp region and the interaction was competed by unlabeled homologous oligonucleotides, but not by mutated competitor DNAs (FIG. 4B). GST protein alone did not bind DNA in control experiments. The ΔLIM pLhx3 protein was cleaved from the GST moiety by thrombin digestion. Pure ΔLIM pLhx3 protein also specifically bound the porcine αGSU site (FIG. 4B). Further, GST fusion proteins containing pLhx3 bound to the porcine αGSU site at significantly lower affinities than LIM-truncated pLhx3 proteins, indicating suppression of DNA binding by the LIM domains (FIG. 4B). By quantifying shifted complexes from EMSA reactions containing equivalent amounts of protein, the data disclosed demonstrate that deletion of the LIM domains increases the affinity of pLhx3 for the porcine αGSU site at least fifteen-fold.

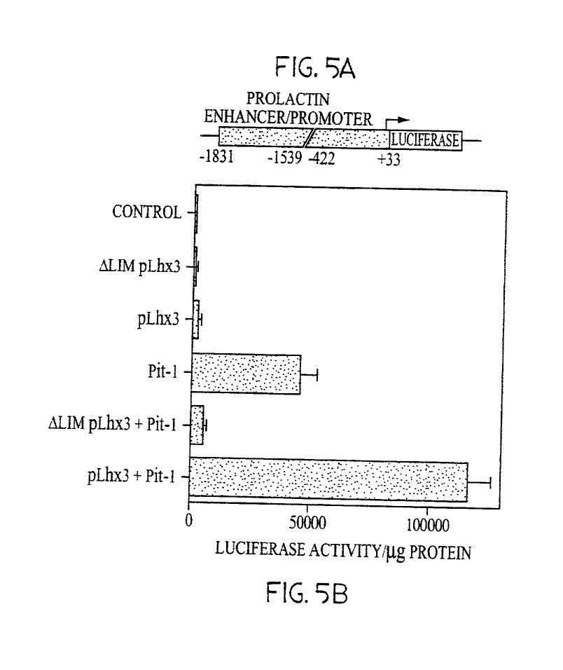

To examine whether porcine Lhx3 could activate the prolactin enhancer/promoter regulatory region, either alone or in combination with Pit-1, co-transfection assays were performed using luciferase reporter genes as described previously elsewhere herein. These experiments demonstrated that pLhx3 could moderately activate prolactin regulatory regions alone and that pLhx3 acted synergistically with Pit-1 in induction of this gene (FIG. 5). Expression vectors for porcine Lhx3 lacking the LIM domains did not significantly activate prolactin promoter reporter genes and were unable to synergize with Pit-1 (FIG. 5). These data suggest that the LIM domains are required for transcriptional synergy and mediate interaction with Pit-1 (see also Bach et al., 1995, Proc. Natl. Acad. Sci. USA 92:2720-2724). Interestingly, ΔLIM pLhx3 inhibited Pit-1 activation of the prolactin gene (FIG. 5).

Interaction of Porcine Lhx3 With Pit-1 and NLI/Ldb1/CLIM Proteins

Murine Lhx3 has been demonstrated to interact with Pit-1 and with the NLI/Ldb1/CLIM family of proteins (Agulnick et al., 1996, Nature 384:270-272; Bach et al., 1995, Proc. Natl. Acad. Sci. USA 92:2720-2724; Bach et al., 1997, Genes Dev. 11:1370-1380; Jurata et al. 1998, J. Biol. Chem. 273:3152-3157). It was determined whether pLhx3 could interact with Pit-1 and NLI using in vitro protein/protein interaction assays.

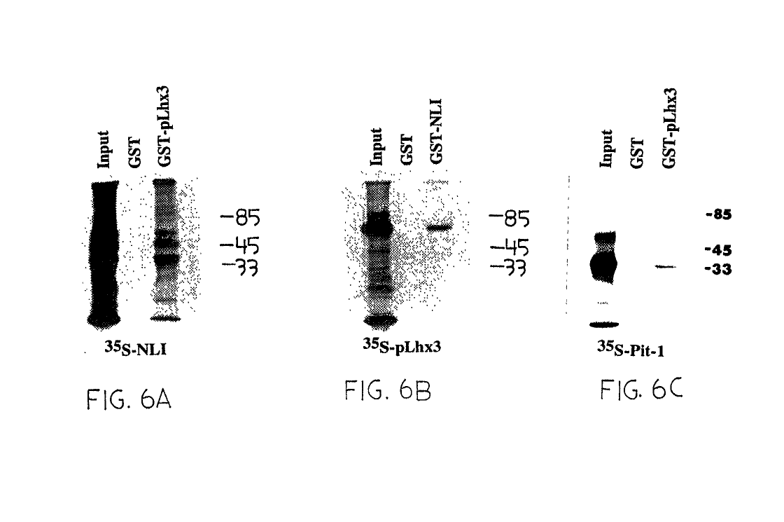

In vitro translation of a plasmid containing the murine NLI cDNA produced a predominant radiolabeled product of approximately 43 kDa and two less abundant forms of the NLI protein. The radiolabeled NLI protein specifically bound to affinity columns containing GST-pLhx3 fusion protein, but not to GST controls (FIG. 6). Translation of pLhx3 RNAs in vitro produced a labeled protein species of approximately 50 kDa, similar in apparent molecular weight to the protein detected in pLhx3-transfected cells (FIG. 3). Radiolabeled pLhx3 protein specifically bound to GST-NLI fusion protein columns, but not to GST controls. Similar experiments demonstrated interaction between pLhx3 and rat Pit-1 (FIG. 6).

LIM proteins are key regulators of developmental pathways. For example, muscle LIM protein (MLP) is important for the differentiation of striated muscle tissues such as the heart (Arber et al., 1997, Cell 88:393-403); Lim1/Lhx1 is required for the development of anterior structures (Shawlot and Behringer, 1995, Nature 374:425-430); Isl- 1-deficient mice exhibit defects in development of motor neurons and the pancreas (Pfaff et al., 1996; Cell 84:309-320; Ahlgren et al., 1997, Nature 385:257-260) and the Lhx2/LH-2 gene is required for eye, forebrain and hematopoietic development (Porter et al., 1997, Development 124:2935-2944). The failure of anterior and intermediate pituitary development in Lhx3 null animals (Sheng et al., 1996; Science 272:1004-1007; Sheng et al., 1997, Science 278:1809-1812) establishes the importance of Lhx3 in mammalian pituitary organogenesis. The data disclosed herein demonstrate that Lhx3 is conserved and expressed in the pig pituitary gland. The amino acid sequence of pLhx3 is highly related to Lhx3 family proteins in other species and pLhx3 can activate pituitary trophic hormone promoters, both alone and in combination with the Pit-1 pituitary transcription factor.

Without wishing to be bound by any particular theory, the conservation of the amino terminal LIM domains and the homeodomain of Lhx3 indicate that these structures are central to its function. The Lhx3 LIM 2 domain and the homeodomain are especially conserved in many types of vertebrates (FIG. 1). Although reports of protein/protein interaction experiments assessing the importance of individual LIM motifs, within Lhx3 and other LIM homeodomain proteins, vary somewhat in their conclusions, there now appears to be consensus that the strongest interactions occur when both LIM motifs are present. For example, the LIM domains of Lhx3 mediate interaction with the NLI family of proteins (Agulnick et al., 1996; Nature 384:270-272; Bach et al., 1997; Genes Dev. I 1:1370-1380; Jurata et al., 1998, Curr. Topics Micro. Immunol. 228:75-113); with the P-Otx/Ptx1 pituitary transcription factor (Bach et al., 1997, Genes Dev. 11:1370-1380); and with the Isl-1/-2 LIM homeodomain factors (Jurata et al. 1998, J. Biol. Chem. 273:3152-3157). The LIM 2 domain of Lmx1, a LIM homeodomain protein, has higher affinity than the LIM 1 domain for NLI and determines specificity in cooperation with the helix-loop-helix factor E47, effecting synergistic transcriptional regulation of the insulin gene (Johnson et al., 1997; Mol. Cell. Biol. 17:3488-3496; Jurata and Gill, 1997, Mol. Cell. Biol. 17:5688-5698).

The role of the LIM domains in individual LIM homeodomain proteins clearly varies. Whereas the data disclosed elsewhere herein demonstrate that the LIM domains of Lhx3 inhibit the binding affinity of the protein to target DNA sites, others have demonstrated that deletion of the LIM domains of Lmx1 does not significantly affect its recognition of DNA sequences in the rat insulin mini-enhancer element (Jurata and Gill, 1997, Mol. Cell. Biol. 17:5688-5698). The functional effects of deletion of the LIM domains have been examined. Mouse Lhx3 lacking both LIM domains is able to activate the murine αGSU promoter, but is unable to synergize with Pit-1, presumably due to loss of the LIM domains which are the interface for interaction with the Pit-1 protein (Bach et al., 1995, Proc. Natl. Acad. Sci. USA 92:2720-2724; Bach et al., 1997, Genes Dev. 11:1370-1380). The data disclosed herein demonstrate that porcine Lhx3 lacking LIM domains can also activate both murine and porcine αGSU promoter reporters, but at reduced efficiency (FIG. 3). The LIM-truncated porcine Lhx3 protein, however, compromised Pit-1 activation of the prolactin gene (FIG. 5). The mechanism of this inhibition is unclear. Like murine Lhx3, porcine Lhx3 lacking the LIM domains has increased affinity for DNA binding sites (FIG. 4). Without wishing to be bound by any particular theory, inhibition of Pit-1 activation of the prolactin enhancer/promoter may be due to non-specific occupation of Pit-1 binding sites within these regulatory regions. Alternately, the differences in function of the murine and porcine Lhx3 proteins may reflect distinct activities of the LIM domains or other regions of the molecule in each species.

In many species the Lhx3 gene is expressed during the development of important regions of the brain and spinal cord. These include neural and neuroendocrine structures such as the pineal gland, retina, hindbrain, spinal cord and pituitary in zebrafish and frogs (Taira et al., 1993; Dev. Biol. 159:245-256; Glasgow et al., 1997, Dev. Biol. 192:405-419). In the chick, expression of combinations of a battery of LIM homeodomain factors, including Lhx3, defines specific classes of motor neurons within the developing spinal cord (Tsuchida et al., 1994, Cell 79:957-970). Without wishing to be bound by any particular theory, the homologies and divergences between the porcine Lhx3 protein sequence and those of other species may therefore also reflect common and distinct functions in these other tissues, as well as within the pituitary gland and its axes. Further studies of Lhx3 and related factors will extend understanding of the pathways that establish the hormone-releasing cells of the pituitary gland and will facilitate therapies designed to treat pituitary diseases and will guide agricultural protocols aimed at improving food animal growth and reproductive fitness.

### EXAMPLE 2

### Identification and Characterization of Porcine Isoforms of Lhx3

The entire coding sequence of porcine Lhx3 was identified and characterized. The data disclosed herein demonstrate that, like human Lhx3, the porcine Lhx3 gene is expressed as two isoforms designated pLhx3a and pLhx3b. The full nucleic acid sequence of pLhx3a (SEQ ID NO:13) is depicted in FIG. 27, while the amino acid sequence of pLhx3a (SEQ ID NO:14) is shown in FIG. 28. The nucleic acid encoding pLhx3a demonstrates about 86.25% sequence homology with mouse Lhx3a. The amino acid sequence of pLhx3a shares approximately 90.25% homology with mouse Lhx3a.

The nucleic acid sequence of pLhx3b (SEQ ID NO:15) is depicted in FIG. 29, while the amino acid sequence of pLhx3b (SEQ ID NO:16) is shown in FIG. 30. The nucleic acid encoding pLhx3b demonstrates about 87.5% sequence homology with mouse Lhx3b. The amino acid sequence of pLhx3a shares approximately 92.8% homology with mouse Lhx3b.

Therefore, the data disclosed herein demonstrate, for the first time, that similarly to humans, which is more fully set forth below, porcine Lhx3 is expressed as at least two isoforms, now designated pLhx3a and pLhx3b.

### EXAMPLE 3

### Identification and Characterization of the Human Lhx3 Transcription Factor

The experiments presented in this example may be summarized as follows.

LIM homeodomain transcription factors are homeobox proteins containing two cysteine-rich zinc finger-like LIM motifs in the N-terminus that mediate protein-protein interactions and a DNA-binding homeodomain in the C-terminus. Lhx3/LIM-3/P-Lim is a LIM homeodomain transcription factor whose expression is critical to the development of the anterior and intermediate lobes of the pituitary gland. Adult expression of Lhx3 has been suggested to play a role in maintaining cellular differentiation of the mature pituitary and may trans-activate the α-glycoprotein subunit and prolactin genes.

The data disclosed herein demonstrate the identification and characterization of human Lhx3 (hLhx3) cDNA and show that hLhx3 comprises two LIM domains and homeodomain that are highly conserved across species. In addition, the data disclosed herein demonstrate that Lhx3 is expressed in the human pituitary. Further, a “real time” quantitative reverse transcription (RT)-PCR assay utilizing the commercially available TaqMan fluorogenic detection system to study the mRNA expression level of Lhx3 in normal and neoplastic pituitary tissues is disclosed.

The Materials and Methods used in the experiments presented in this example are now described.

Identification of Human Lhx3 Gene

Computer analysis of the National Center for Biotechnology Information databases using the BLAST program and the mouse Lhx3 DNA sequence as a query, identified a single unidentified human expressed sequence tag (EST) that appeared to encode a related protein. The EST was unavailable since the original stocks were not viable (i.e., commercial vendors could not grow the strains); therefore, oligonucleotide primers I33 (cggaattctacaacacctcgcccaagccgg [SEQ ID NO:70]) and I34 (cggaattcggaacgaggggcccttgac [SEQ ID NO:71]) were designed and used to amplify this sequence from human pituitary cDNA using the polymerase chain reaction (PCR).

The fragment was cloned into pBluescript KS-II (Stratagene, La Jolla, Calif.) using standard techniques. The clone was completely sequenced on both strands using the Sanger dideoxy method and Sequenase (Amersham/USB). Sequence analysis and alignment were performed using Wisconsin Genetics/GCG and DNASIS (Hitachi, San Jose, Calif.) software.

PCR and oligonucleotide primers I33 (SEQ ID NO:70) and I47 (gatctagtacaaatatgtaagtatag) were used to generate a 700 bp Lhx3 fragment using a human anterior pituitary cDNA RACE (Rapid Amplification of cDNA ends) library (ClonTech Inc. Palo Alto, Calif.). This cDNA was cloned into the A/T pGEM-T Easy plasmid (Promega Corp., Madison, Wis.) and sequenced as above. DNA sequence analysis enabled the design of oligonucleotide I52 (gatccaaaaggaaatgagaga [SEQ ID NO:72]), which was used in combination with 5′ RACE anchor primers, AP1 and AP2, which are commercially available as part of a RACE kit (ClonTech Inc. Palo Alto, Calif.), to amplify an additional 500 bps of the human Lhx3 cDNA. Primers 1191 (cagtgcaggtggtacacgaagtcct) [SEQ ID NO:73] and I192 (cagtgcaggtggtacacgaagtcct) [SEQ ID NO:74] were designed from the additional Lhx3 DNA sequence data and used in combination with the anchor primers to generate multiple forms of the human Lhx3 cDNA.

The human Lhx3 clone had about 94% identity to mouse Lhx3. The sequence of human Lhx3/P-Lim/LIM-3 was previously unknown. The sequence of human Lhx3 is important for the development of diagnostic tests. In addition, the data disclosed herein demonstrate, as more fully set forth elsewhere herein, that there are multiple forms of this factor present in normal humans, i.e., humans having no evidence of pituitary disease. The presence of isoforms of Lhx3, disclosed herein for the first time, provides crucial information essential for the design and interpretation of appropriate diagnostic tests.

In addition, the genomic DNA encoding human Lhx3/P-Lim/LIM-3 has been cloned and characterized as more fully disclosed elsewhere herein, and the gene has been mapped to the subtelomeric region of human chromosome 9.

Assay of Expression of Human Lhx3

A quantitative “real time” PCR assay was developed to determine human Lhx3 gene expression levels in human tissues such as pituitary adenomas. This permits the quantification of human Lhx3 gene expression. Abnormal (high or low) levels of human Lhx3 gene expression may be associated with, or causative for, human disease. Therefore, this quantitative method can be used for diagnosis of altered level of Lhx3 in humans.

The method was performed as follows. An ABI Prism 7700 Sequence Detector was used as described by Sloop et al. (1998, Biochem. Biophys. Res. Comm. 251:142-147). More specifically, 0.75-3 μg of total RNA from human tissues were assessed for hLhx3 expression using RT-cDNA synthesis. Oligonucleotides I104 (ggacaaggacagcgttcag [SEQ ID NO:75]) and I105 (ctcccgtagaggccattg [SEQ ID NO:76]), which produced a 104 bp amplicon of human Lhx3, were used. Further, sequence 193 (ttccccgatgagccttccttggcggaa [SEQ ID NO:77]) was used as an internal fluorescent oligonucleotide probe (i e. a TaqMan probe). The internal fluorescent oligonucleotide probe was commercially prepared and obtained (Perkin Elmer, Norwalk, Conn.) and the “real time” assay was performed per the manufacturer's instructions.

The assay was performed using several human anterior pituitary cDNAs to produce a standard curve and deduced the expression level of human Lhx3 in non diseased human pituitary tissue. Control reactions for β-actin were performed in tandem.

Assay of Genetic Mutations of Human Lhx3

The presented sequence can be used to diagnose, detect, or both, mutations in the hLhx3 gene that may mediate human disease, disorder or condition. Moreover, such mutations can be detected using the polymerase chain reaction/sequencing methods as disclosed herein or as well known in the art.

### EXAMPLE 4

### Differential Activation of Pituitary Hormone Genes by Human Lhx3 Isoforms With Distinct DNA Binding Properties

The experiments presented in this example may be summarized as follows.

Lhx3 is a LIM homeodomain (“HD”) transcription factor essential for pituitary development and motor neuron specification in mice. The data disclosed herein demonstrate the presence of at least two isoforms of human Lhx3, hLhx3a and hLhx3b, which isoforms differ in their ability to trans-activate pituitary gene targets and to bind to specific DNA sequences. These factors are identical within the LIM domains and the homeodomain, but differ in their amino-terminal sequences preceding the LIM motifs.

To further characterize the molecular actions of Lhx3, two alternate forms of human Lhx3, hLhx3a and hLhx3b, which share the LIM domains, homeodomain, and carboxyl terminus of Lhx3, but possess distinct amino-terminal protein sequences, were identified and characterized. Both isoforms are nuclear proteins and are detected in the pituitary gland, but trans-activation assays revealed different abilities to activate anterior pituitary hormone gene regulatory regions. Human Lhx3a activated a reporter gene containing the α-glycoprotein subunit (αGSU) promoter and a minimal reporter gene containing consensus Lhx3 binding sites. The data disclosed herein demonstrate that Lhx3a synergized with Pit-1 in induction of the TSH β-subunit (TSH β) gene promoter. By contrast, Lhx3b was either inactive or only weakly activated trophic hormone genes. Further, the data disclosed herein demonstrate that the differences in trans-activation ability between hLhx3a and Lhx3b correlate with their DNA-binding to sites within these target genes. The results disclosed herein indicate that the amino terminus of hLhx3b inhibits the ability of this factor to bind DNA and trans-activate target genes compared with Lhx3a. This is the first description of different functional properties for alternate forms of a LIM homeodomain class transcriptional regulator. Without wishing to be bound by any particular theory, the hLhx3a-specific and hLhx3b specific amino-terminal domains may represent novel functional motifs derived throughout evolution to confer properties unique to Lhx3 that are important in mammalian development. These findings suggest that Lhx3 isoforms perform distinct roles in the development of the pituitary gland and during motor neuron differentiation.

The data disclosed herein further demonstrate that both human Lhx3 isoforms are localized to the nucleus and are expressed in the adult human pituitary, but gene activation studies demonstrate characteristic functional differences. That is, human Lhx3a trans-activated the α-glycoprotein subunit (αGSU) promoter and a reporter construct containing a high-affinity Lhx3 binding site more effectively than the hLhx3b isoform. In addition, hLhx3a synergized with the pituitary POU domain factor, Pit-1, to strongly induce transcription of the thyroid-stimulating hormone beta (TSHβ)-subunit gene, while hLhx3b did not. Moreover, the data disclosed herein demonstrate that the differences in gene activation properties between hLhx3a and hLhx3b correlate with their DNA binding to sites within these genes. The data disclosed herein further demonstrate that the short hLhx3b-specific amino-terminal domain inhibits DNA binding and gene activation functions of the molecule. Without wishing to be bound by any particular theory, these data suggest that isoforms of Lhx3 play distinct roles during development of the mammalian pituitary gland and other neuroendocrine systems.

The Materials and Methods used in the experiments presented in this example are now described.

RNA Extraction /cDNA Synthesis

Adult human pituitaries were obtained from the National Hormone and Pituitary Program or were obtained at autopsy at the Indiana University School of Medicine. As could best be assessed, pituitary samples were normal and did not have adenomas. Patients ranged in age from 36 to 65 years, and weighed from 75 to 136 kg. Tissues were frozen in liquid nitrogen and were ground to a powder on dry ice/liquid nitrogen. Total RNA was extracted from the samples using Tri-Reagent (Molecular Research Center, Inc., Cincinnati, Ohio) and bromochloropropane as a chloroform substitute. cDNA was synthesized using Superscript II reverse transcriptase (Life Technologies, Inc., Gaithersburg, Md.), oligo d(T), random hexamer, and gene-specific primers, as required.

DNA Cloning/DNA Sequencing/Plasmid Construction

Database searches revealed that a human EST sequence displayed similarity to murine and porcine Lhx3. The bacterial culture containing this plasmid could not be retrieved by any of the distributors of EST clones. PCR was then used to amplify this sequence from adult human pituitary cDNA using the following primers:

| | |
|---|---|
| 5′-cggaattctacaacacctcgcccaagccgg-3′ | [SEQ ID NO:78] and |
| 5′-cggaattcggaacgaggggcccttgac-3′ | [SEQ ID NO:79]. |

RACE was then performed to obtain the full-length cDNA encoding human Lhx3 using the 5′-RACE 2.0 system (Life Technologies, Inc., Gaithersburg, Md.) and adult human pituitary cDNA. Finally, partial hLhx3 cDNAs were used as high stringency probes (final wash=0.5× SSC, 65° C.) of a human pituitary cDNA library (ClonTech Laboratories, Inc., Palo Alto, Calif.). From one million plaques assayed, 12 positive bacteriophage were identified, and cDNAs from these phage were subcloned into pBluescript KS II(−) (Stratagene, La Jolla, Calif.) as described previously (Meier et al., 1999, Mol. Cell. Endocrinol. 147:65-74).

Of the 12 clones isolated, seven clones were completely sequenced on both strands by automated DNA sequencing using a Perkin Elmer Corp. (Norwalk, Conn.) DNA sequencer at the Biochemistry Biotechnology Facility, Indiana University School of Medicine. Sequence analysis was performed using Wisconsin Genetics/GCG and DNASIS (Hitachi, San Jose, Calif.) software.

Expression vectors for hLhx3a and hLhx3b were generated by directly cloning full-length cDNAs into pcDNA3 (Invitrogen, Carlsbad, Calif.). Myc epitope-tagged hLhx3 expression vectors (hLhx3a-myc, hLhx3b-myc) were prepared by cloning compatible cDNA fragments into pcDNA3.1/Myc-His(−)C (Invitrogen). DNA fragments ere generated by PCR using the following primers:

| | | |
|---|---|---|
| 5′-cgggatccgatcgcttcggcagcagctg-3′ | (5′, hLhx3a-myc | [SEQ ID NO:80]); |
| 5′-cgggatccttgatatttaccccggaggc-3′ | (5′hLhx3b-myc | [SEQ ID NO:81]); and |
| 5′-gcgaagcttggaactgagcgtggtctacctca-3′ | (3′, hLhx3a/b-myc | [SEQ ID NO:82]). |

An expression vector containing four copies of (green fluorescence protein (GFP) was constructed by removing the 4xGFP cassette from plasmid p703 (Chatterjee et al., 1997, Exp. Cell. Res. 236:346-350) by digestion with KpnI and EcoRI and cloning of this fragment into pcDNA3 to generate pcDNA3-4xGFP. Compatible hLhx3 DNA fragments were generated by PCR using the following primers:

| | |
|---|---|
| 5′-tacaagcttcgcgatgctgctggaaacgg-3′ | (5′, hLhx3a [SEQ ID NO:83]); |
| 5′-tacaagcttaccatggaggcgcgcgggga-3′ | (5′, hLhx3b [SEQ ID NO:84]); and |
| 5′cccggtaccaactgagcgtggtctacctc-3′ | (3′, hLhx3a/b [SEQ ID NO:85]). |

These fragments then were digested with HindIII and KpnI and cloned into pcDNA3-4xGFT to generate vectors expressing hLhx3a-4xGFP and hLhx3b-4xGFP.

Southern and Northern Analyses

Human genomic DNA was extracted from peripheral blood using a QIAamp Blood Maxi Kit (Qiagen, Chatsworth, Calif.). DNA was digested to completion with restriction enzymes, electrophoresed through 0.7% agarose gels, and transferred to nylon membranes (Hybond-N+, Amersham Pharmacia Biotech, Piscataway, N.J.). Membranes were hybridized to radiolabeled hLhx3 cDNA probes in Express Hyb buffer (ClonTech Laboratories, Inc., Palo Alto, Calif.) at 1-2×10⁶ cpm/ml for 24 hours. DNA fragments were labeled by random priming using the Klenow fragment enzyme (Life Technologies, Inc., Gaithersburg, Md.) and ³²P-dCTP (Amersham Pharmacia Biotech) to a specific activity of greater than about 1×10⁹ cpm/μg. After hybridization, the membranes were washed in 0.5× SSC at 65° C., followed by exposure to MR film (Eastman Kodak Co., Rochester, N.Y.) with intensifying screens.

Total human pituitary RNA was extracted as described previously elsewhere herein and poly A⁺ RNA was purchased from ClonTech Laboratories, Inc. (Palo Alto, Calif.). RNA was separated on denaturing, formaldehyde agarose gels followed by transfer to Nytran Plus membranes using the Turboblotter system (Schleicher & Schuell, Inc., Keene, N.H.). Probes used were a 0.4 kb hLhx3 cDNA fragment encoding the LIM domains of the protein and a 1.2 kb cDNA encoding human Pit-1. Membranes were hybridized to radio-labeled cDNA probes as described previously elsewhere herein.

In vitro Transcription/Translation

Radiolabeled hLhx3 proteins were synthesized in vitro from pcDNA3 expression vector substrates using T7 RNA polymerase, TnT rabbit reticulocyte lysates (Promega Corp., Madison, Wis.), and ³⁵S-methionine (Amersham Pharmacia Biotech, Arlington Heights, Ill.). Radiolabeled proteins were analyzed using SDS-PAGE followed by treatment with Amplify fluorography reagent (Amersham Pharmacia Biotech) and exposure to MR film (Eastman Kodak Co., Rochester, N.Y.) at −80° C.

Confocal Microscopy

Human 293T cells (1×10⁵) were grown in chamber slides (Nunc) and transfected with 4xGFP, hLhx3a-4xGFP, or hLhx3b-4xGFP expression vectors as described below. After 48 hours, the cells were washed with 2× PBS, and then either directly visualized live or fixed with 2% paraformaldehyde, washed with 70% ethanol/30% PBS, and stored in 1× PBS before visualization. Fluorescence and trans-illumination images were collected using a MRC 1024 laser scanning confocal microscope (Bio-Rad Laboratories, Inc., Richmond, Calif.) with a 60× water immersion objective (Nikon, Melville, N.Y.) and a krypton-argon laser. Images were captured with Metamorph software (Universal Imaging, West Chester, Pa.).

Quantitative Assay of Gene Expression

“Real time” quantitative PCR utilizing the ABI PRISM 7700 Sequence Detection System (Perkin Elmer Corp., Norwalk, Conn.) was performed as previously described (Sloop et al., 1998, Biochem. Biophys. Res. Commun. 251:142-147; Held et al., 1996, Genome Res. 6:986-994). More specifically, total RNA was isolated from human pituitary tissue as described previously elsewhere herein. Reverse transcription of total RNA using a hLhx3-specific reverse primer (5′-ctcccgtagaggccattg-3′ [SEQ ID NO:______]) was performed using SuperScript II reverse transcriptase (Life Technologies, Inc., Gaithersburg, Md.). Quantitative PCR amplification reactions were performed in triplicate and included: 2 μl of cDNA synthesis reaction, IX TaqMan Buffer A, 300 nM dATP, dCTP, dGTP, and 600 nM dUTP, 3.5 mM MgCl₂, 1.25 U AmpliTaq Gold DNA polymerase (Perkin Elmer Corp., Norwalk, Calif.), 0.5 U of AmpErase uracil-N-glycosylase, 300 nM forward primer (5′-ggacaaggacagcgttcag-3′ [SEQ ID NO:86]) and reverse primer (5′ctcccgtagaggccattg-3′ [SEQ ID NO:87]), and 200 nM hLhx3 fluorogenic probe (5′-ttccccgatgagccttccttggcggaa-3′ [SEQ ID NO:40]). Reaction parameters were as follows: 50° C., 2 minutes; 94° C., 10 minutes; and then 35 cycles of 95° C., 30 seconds 60° C., 1 minute. Serial dilutions of hLhx3 cDNA were amplified simultaneously with patient samples to generate a standard curve. Values reported are averages of four independent experiments.

RT-PCR Analysis

cDNA was synthesized from adult pituitary RNA as described above using oligo-d(T) as a primer. PCR then was performed using the following primers:

| | |
|---|---|
| 5′-cgggatccatgctgctggaaacggggct-3′ | (5′, hLhx3a [SEQ ID NO:88]); |
| 5′-cgggatccatggaggcgcgcggggagct-3′ | (5′, hLhx3b [SEQ ID NO:89]); and |
| 5′-cggaattctcagaactgagcgtggtcta-3′ | (3′, hLhx3a/b [SEQ ID NO:90]). |

The cycling parameters were as follows: 94° C., 30 seconds; 60° C., 30 seconds; 72° C., 1 minute; for 30 cycles. Reaction products were analyzed on 1% agarose Tris-borate gels.

For analysis of mouse pituitary cell lines, cDNA was synthesized from total RNA using 5′-tggtcacagcctgcacacat-3′ [SEQ ID NO:91]). PCR then was performed using the following primers:

| 5′-aaccactggattagtgactg-3′ | (5′, mLhx3a [SEQ ID NO: |
|---|---|
| | 92]); |
| 5′-gaagttcagggtcggaggg-3′ | (5′, mLhx3b [SEQ ID NO: |
| | 93]); and |
| 5′-tggtcacagcctgcacacat-3′ | (3′, mLhx3a/b [SEQ ID NO: |
| | 94]). |

Cycling parameters were as follows: 94° C., 30 seconds; 60° C., 30 seconds 72° C., 30 seconds; for 30 cycles. Reaction products were analyzed on 11% acrylamide Tris-borate gels.

Cell Culture, Transfection Assays, Statistical Analysis

Human embryonic kidney 293 and 293T cells were cultured in DMEM (Life Technologies, Inc., Gaithersburg, Md.) with 10% fetal bovine serum (FBS) (Irvine Scientific, Santa Ana, Calif.), 100 U/ml penicillin, and 100 μg/ml streptomycin (Irvine Scientific). Approximately 1.5×10⁵ cells per 60-mm dish were transfected with calcium phosphate/DNA precipitates using the CalPhos system (ClonTech Laboratories, Inc., Palo Alto, Calif.). Reporter plasmid (0.5 μg) and expression vector (0.1-1.0 μg) were added per 60-mm dish, and all groups received equal final DNA concentrations.

The murine αGSU promoter luciferase plasmid as described previously (Roberson et al., 1994, Mol. Cell Biol. 14:2985-2993) and the murine TSHβ-1.2 kb promoter luciferase plasmid as described by Drolet et al. (1991, Genes Dev. 5:1739-1753) were used. The Lhx3 consensus binding site reporter gene was constructed by cloning three copies of 5′-cagaaaattaattaattgtaa-3′ [SEQ ID NO:95] upstream of a minimal PRL (−36bp) promoter luciferase reporter gene (Bridwell et al., in preparation). Control cultures received empty expression vector DNA. Luciferase activity was measured 48 hours after transfection as described previously (Meier et al., 1999, Mol. Cell Endocrinol. 147:65-74).

All assays were performed in triplicate. Total cell protein was determined by the Bradford method (Bio-Rad laboratories, Inc.) and luciferase activity was normalized to protein concentration. Data points were compared using a one-tailed Student's t test for paired samples using Sigma Plot 2.0 (Jandel Scientific, San Rafael, Calif.). Values were considered significantly different when P<0.05.

Western Blot Analysis

Western blot analysis of 293 or 293T cells transfected with hLhx3a-myc, hLhx3b-myc, or control expression vectors was performed as described previously (Meier et al., 1999, Mol Cell Endocrinol. 147:65-74). The mouse anti-myc monoclonal antibody 9E10, developed by Evan et al. (1985, Mol Cell Biol. 5:3610-3616), was obtained from the Developmental Studies Hybridoma Bank developed under the auspices of the National Institute of Child Health and Human Development and maintained by the Department of Biological Sciences, University of Iowa (Ames, Iowa). Ascites fluid was used at a 1:5,000 dilution.

The secondary antibody was a goat anti-mouse conjugated with horseradish peroxidase (Sigma, St. Louis, Mo.) used at a 1:15,000 dilution. The results were visualized using Supersignal chemiluminescence reagents (Pierce Chemical Co., Rockford, Ill.) and MR film (Eastman Kodak Co., Rochester, N.Y.). Data were quantified using a Bio-Rad Laboratories, Inc., imaging densitometer and Molecular Analyst software (Bio-Rad Laboratories, Inc., Richmond, Calif.).

Recombinant Protein Preparation/EMSA

Bacterial expression vectors for GST-hLhx3a and GST-hLhx3b fusion proteins were generated by cloning BamHI/EcoRI compatible fragments of the hLhx3a and hLhx3b cDNAs into pGEX-KT (Hakes and Dixon, 1992, Anal Biochem. 202:293-298). cDNA fragments were generated by PCR using the following oligonucleotides:

| | |
|---|---|
| 5′-cgggatccatgctgctggaaacggggct-3′ | [SEQ ID NO:96], |
| 5′-cggaattctcagaactgagcgtggtcta-3′ | [SEQ ID NO:97](hLhx3a) and |
| 5′-cgggatccatggaggcgcgcggggagct-3′ | [SEQ ID NO:98], |
| 5′-cggaattctcagaactgagcgtggtcta-3′ | [SEQ ID NO:99](hLhx3b). |

Recombinant proteins were expressed in E. coli BL21 (DE3) pLysS and affinity-purified as described previously (Meier et al., 1999, Mol. Cell Endocrinol. 147:65-74). Proteins were analyzed on 12% SDS-PAGE gels.

EMSAs were performed as described previously (Meier et al., 1999, Mol. Cell Endocrinol. 147:65-74). Oligonucleotides representing the −350 to −323 bp region of the murine αGSU promoter (5′-acattaggtacttagctaattaaatgtg-3′ [SEQ ID NO:100] and 5′-cacatttaattagctaagtacctaatgt-3′ [SEQ ID NO:101]) were used or the Lhx3 consensus binding site described previously elsewhere herein. In competition experiments, 1000-fold molar excess of unlabeled binding site DNA was added to EMSA reactions.

The Results of the experiments presented in this example are now described.

Cloning of Human Lhx3 cDNAs

PCR was used to amplify a region encoding the carboxyl terminus of human Lhx3 (hLhx3) from pituitary cDNA as described elsewhere herein. Rapid amplification of cDNA ends (5′-RACE) was then performed to obtain longer cDNAs. Finally, partial hLhx3 cDNAs were used as probes in screens of a pituitary cDNA library to obtain full-length clones.

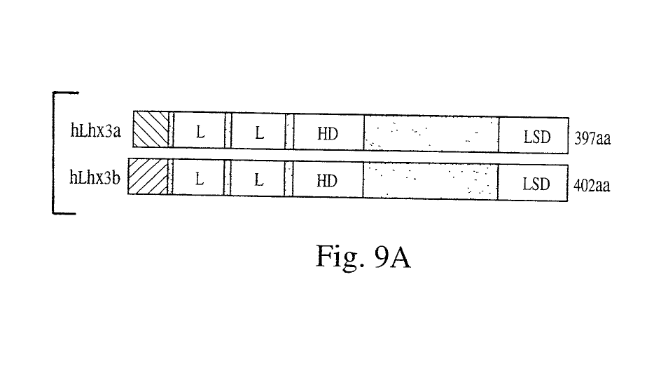

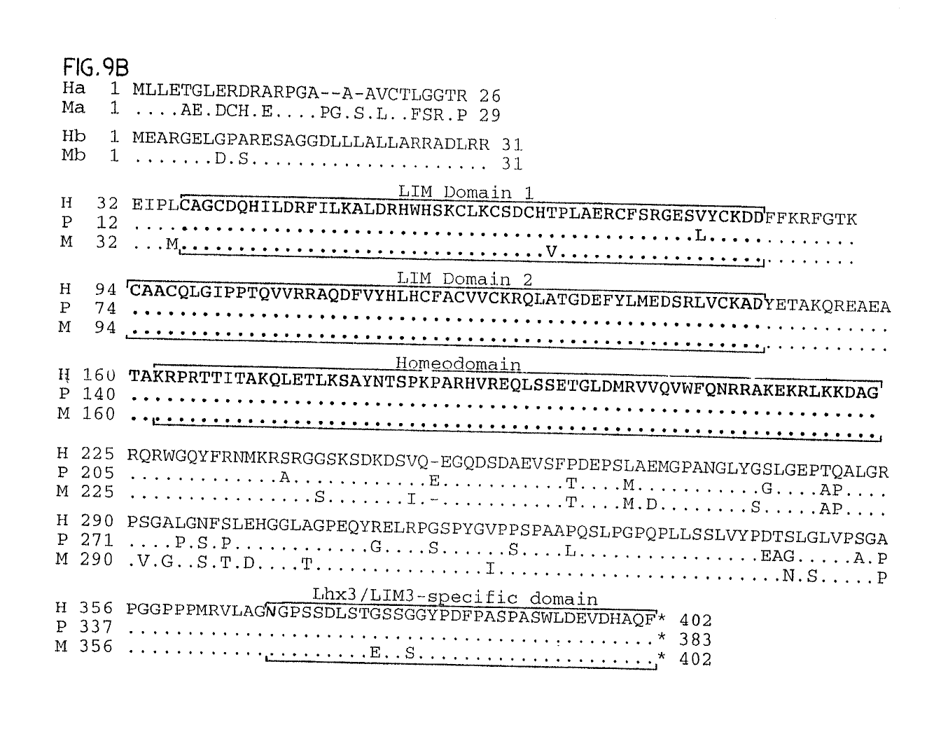

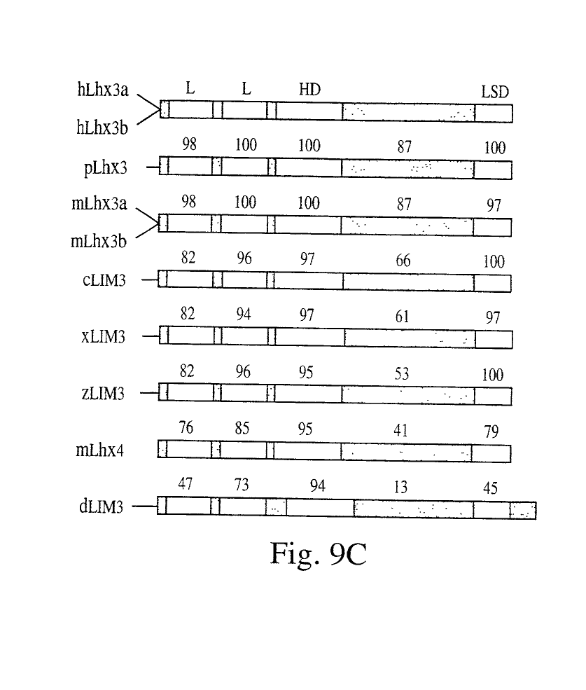

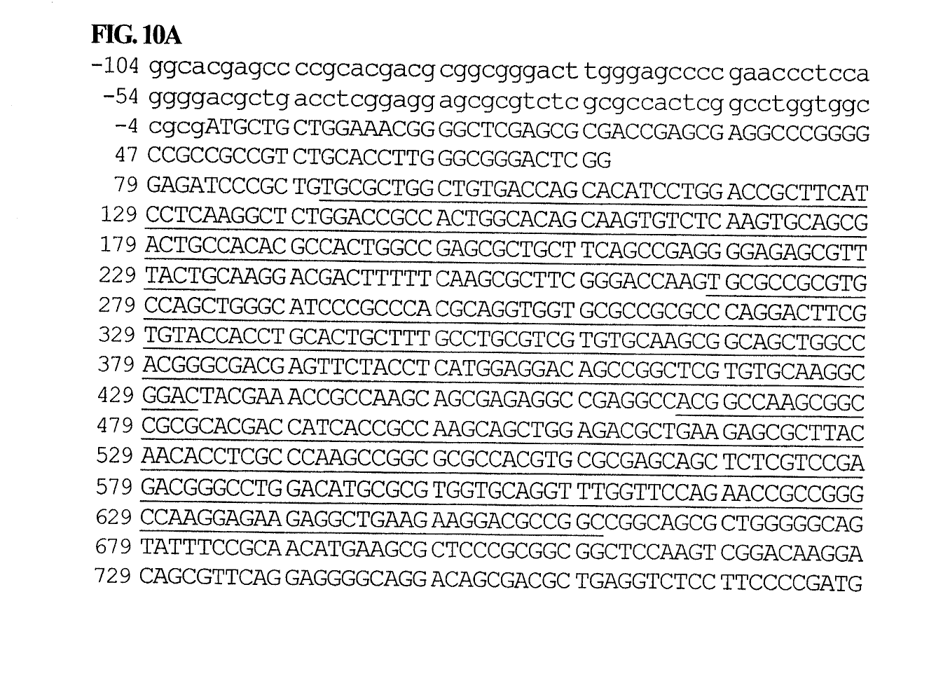

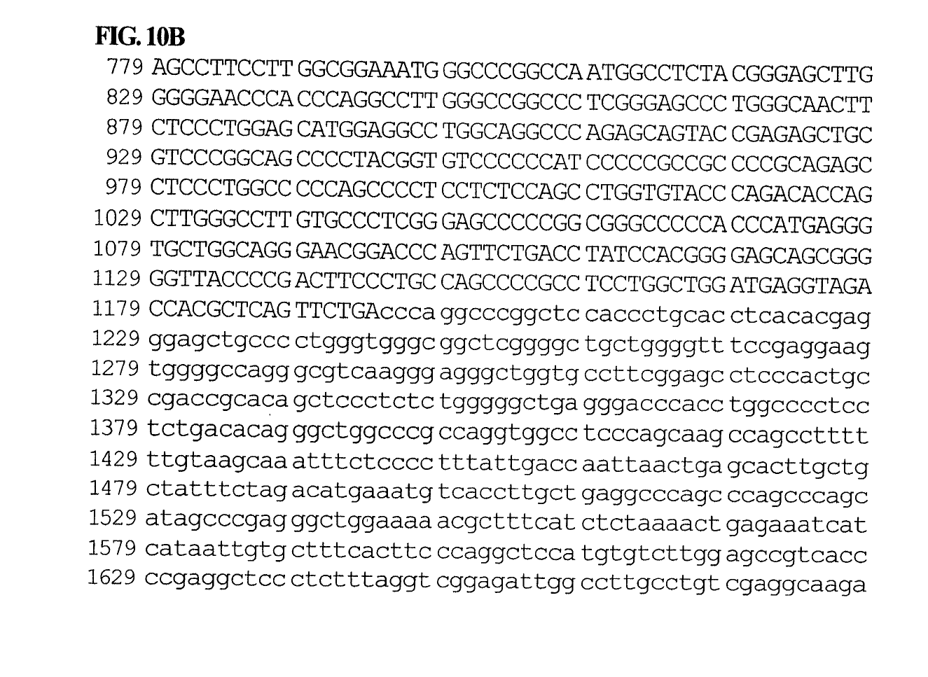

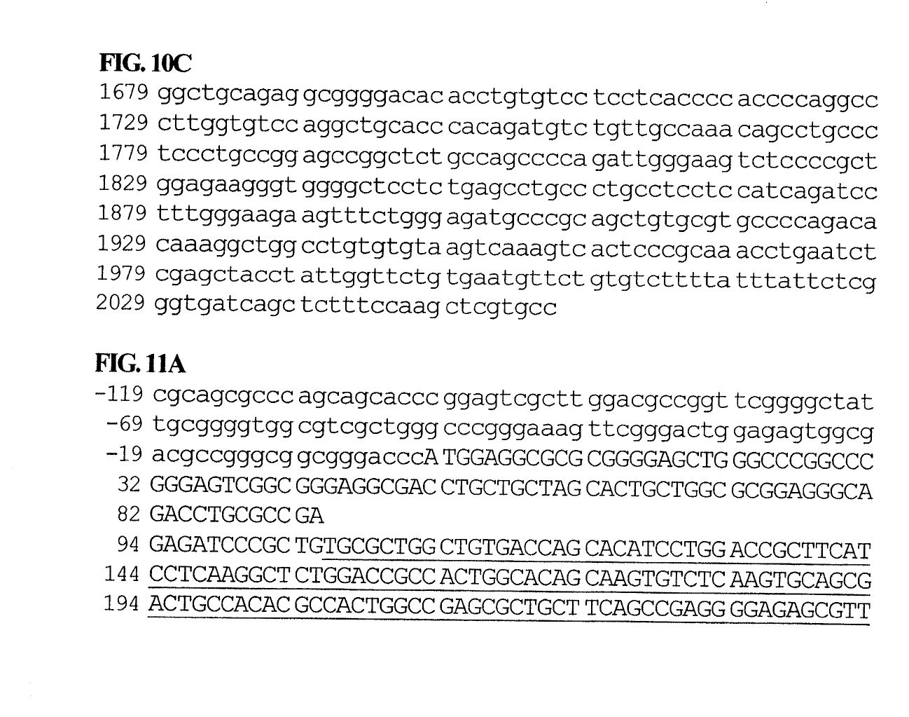

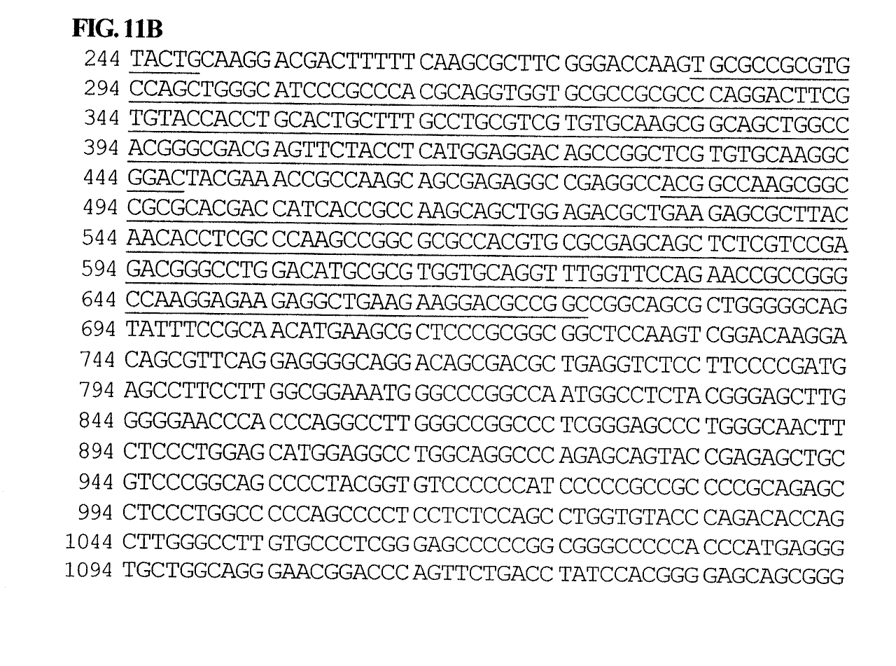

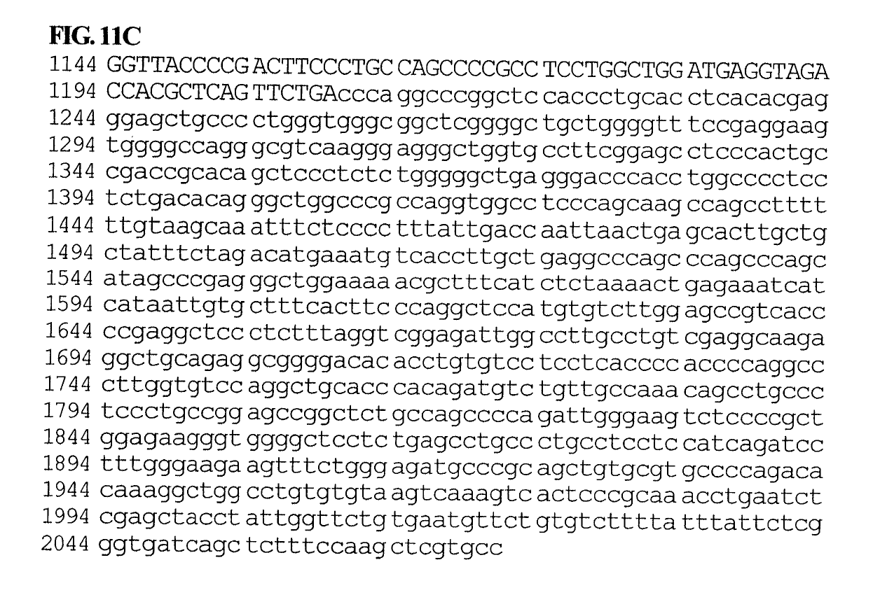

Sequencing of hLhx3 clones obtained from RACE and library screening revealed multiple, distinct hLhx3 cDNAs. Two predominant cDNA species encoded hLhx3 proteins with identical LIM domains, homeodomains, and carboxyl termini, but with distinct amino-terminal protein sequences (FIG. 9A). These protein isoforms have been termed hLhx3a (SEQ ID NO:4) and hLhx3b (SEQ ID NO:6). The nucleic acid sequence of hLhx3a (SEQ ID NO:3) is depicted in FIG. 10, and the nucleic acid sequence of hLhx3b (SEQ ID NO:5) is depicted in FIG. 11. The GenBank accession numbers of the reported sequences are hLhx3a (AF156888) and hLhx3b (AF 156889). Some hLhx3b cDNAs contained an approximately 200-bp insert in the 3′-untranslated region (nucleotide sequences deposited in GenBank) as follows:

| | |
|---|---|
| 5′-tcttccgggagaggccccctcctctccccagaccacagggggcctctctg | |
| cctccagccccaccttccccgggagaagctttccccaatccccaggtctctagat | |
| cattctgttctcgagtatcctgtggaggaggcaaaaatgcctggcgccccttctct | |
| ccaagctcaattctctaagcccctcagggtc-3′ | (SEQ ID NO:102). |

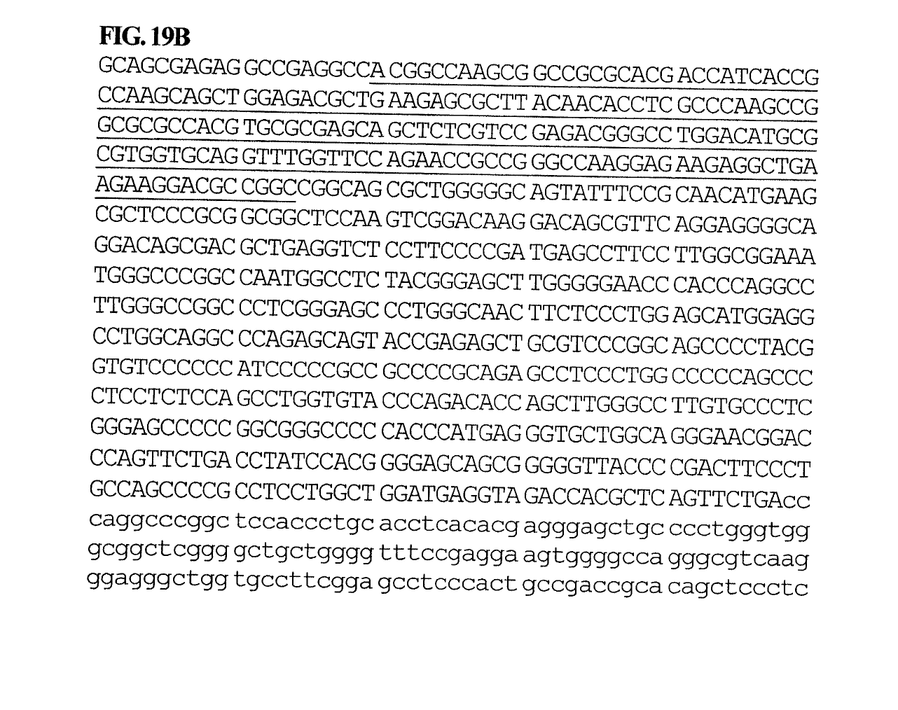

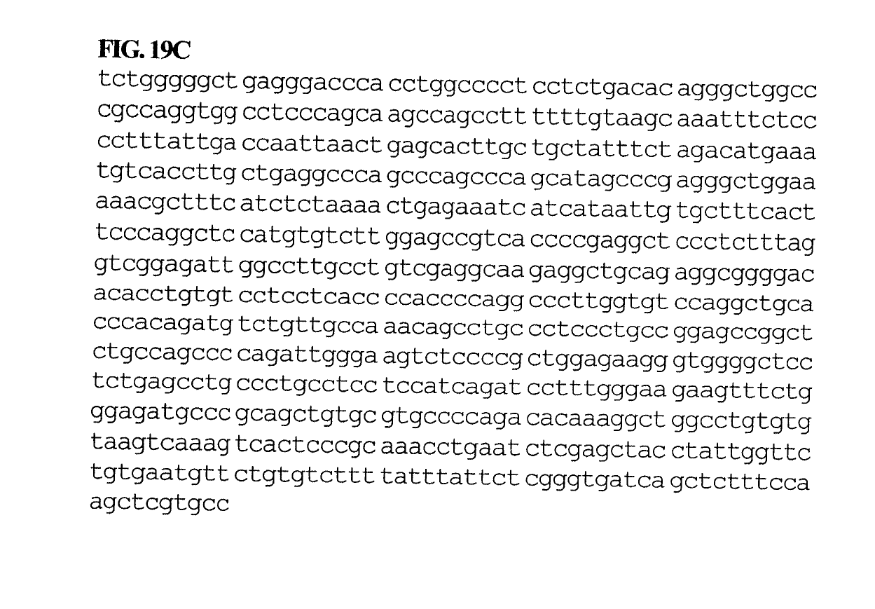

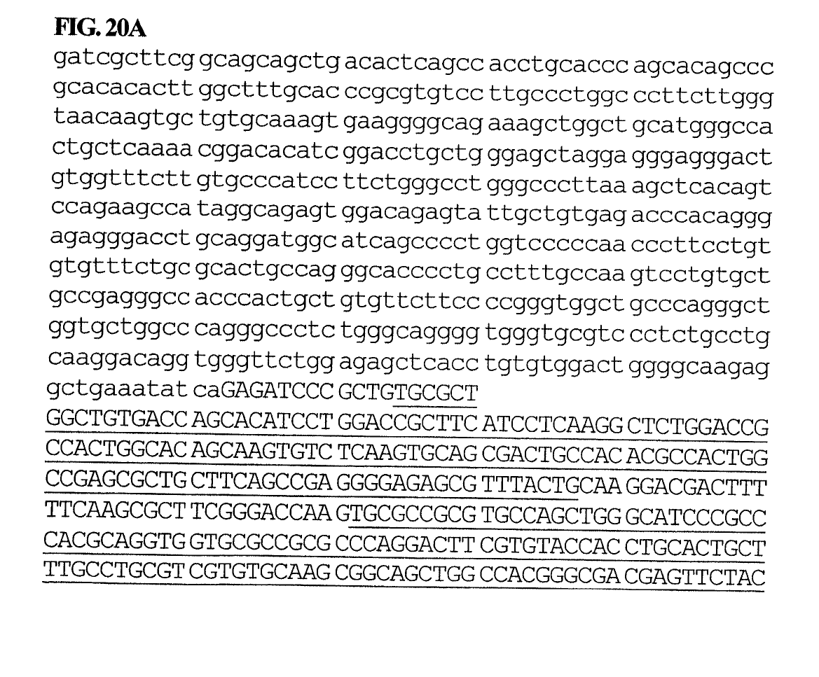

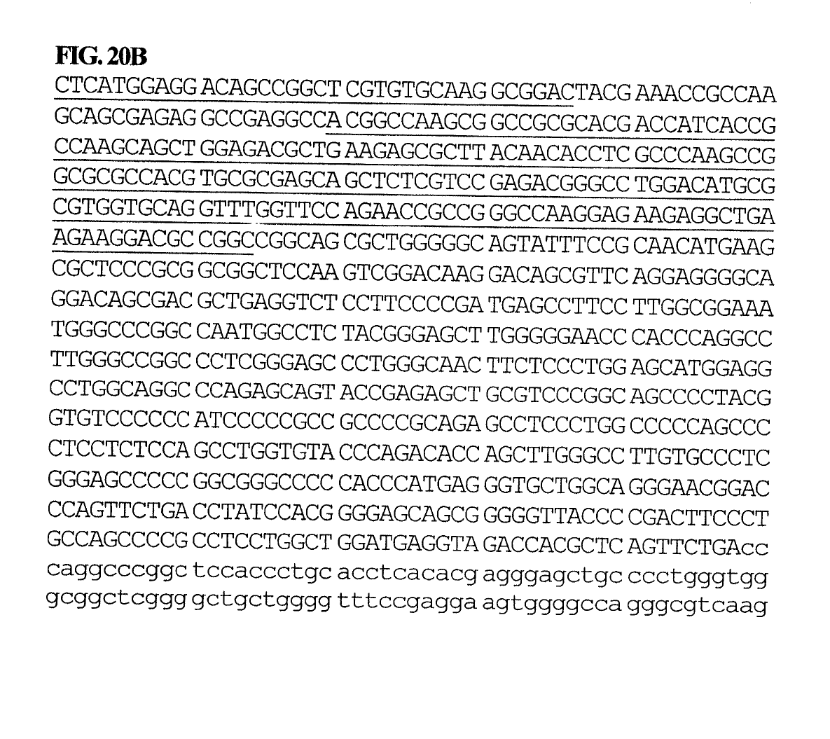

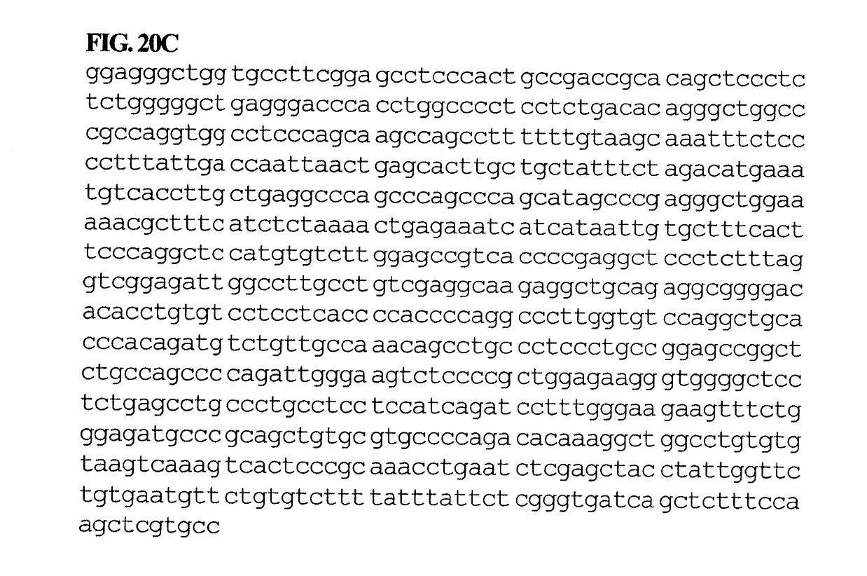

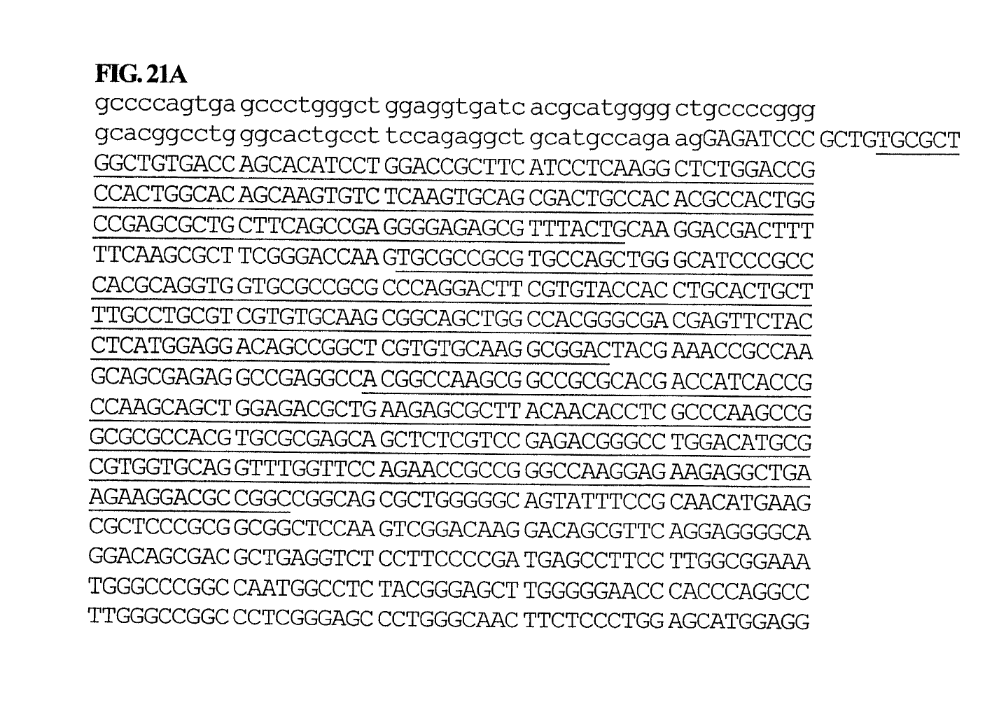

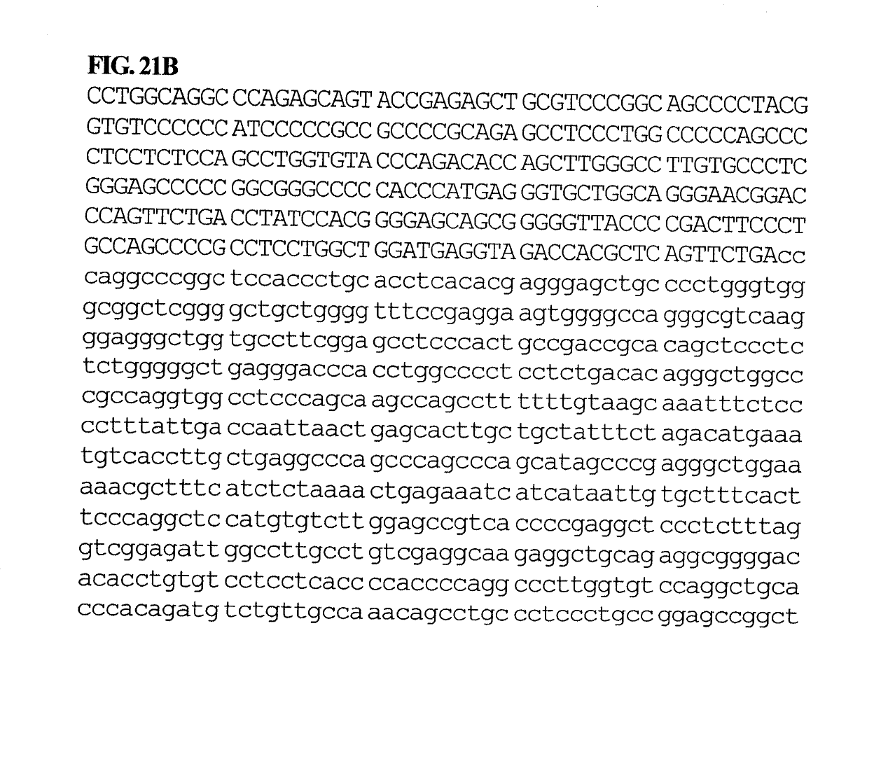

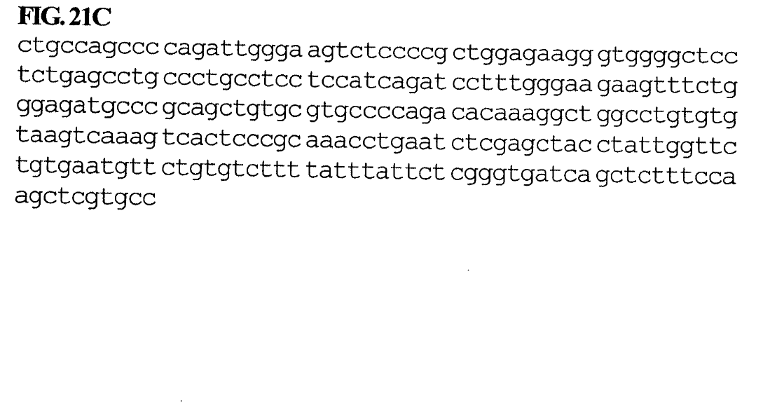

The data disclosed herein also demonstrate a number of other potential human Lhx3 isoforms, including hLhx3c (SEQ ID NO:25) (FIG. 19), hLhx3d (SEQ ID NO:26) (FIG. 20), hLhx3e (SEQ ID NO:27) (FIG. 21).

The unique amino-terminal region of hLhx3b is very similar to an alternate form of murine Lhx3 (Zhadanov et al., 1995, Dev. Dynam. 202:354-364), ie., 94% identity (FIG. 9B). By contrast, the shorter amino-terminal sequence of hLhx3a is only 58% similar to the amino terminus of murine Lhx3a (FIG. 9B). The a- and b-specific domains of hLhx3 do not significantly match other sequences in the databases searched.

The common regions of the hLhx3 proteins display strong overall similarity to murine (94% identity) and porcine (95% identity) Lhx3 sequences (FIG. 9B). The LIM and DNA binding domains of Lhx3 are strongly conserved in the mammalian species, with complete identity in the LIM 2 domain and the homeodomain (FIG. 9B). Comparison of mammalian Lhx3 sequences with Lhx3/LIM3 family proteins from non-mammalian species also reveals conservation of the LIM and DNA-binding domains and the carboxyl-terminal LIM3-specific domain (LSD) noted by Glasgow et al. (1997, Nature 397:76-80) and Thor et al. (1999, Nature 397:76-80) (FIG. 9C). The amino termini of non-mammalian Lhx3/L1 μM3 proteins display more similarity to the amino terminus of hLhx3a than to that of hLhx3b, but overall are diverged in length, composition, and sequence.

Analysis of the Human Lhx3 Gene and its Products

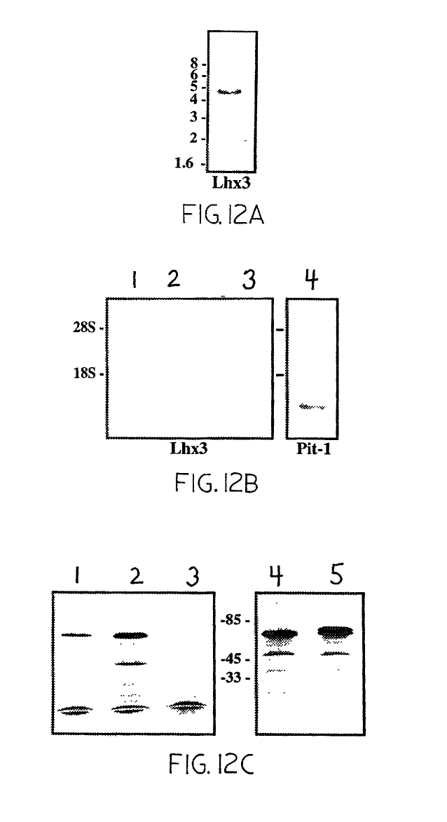

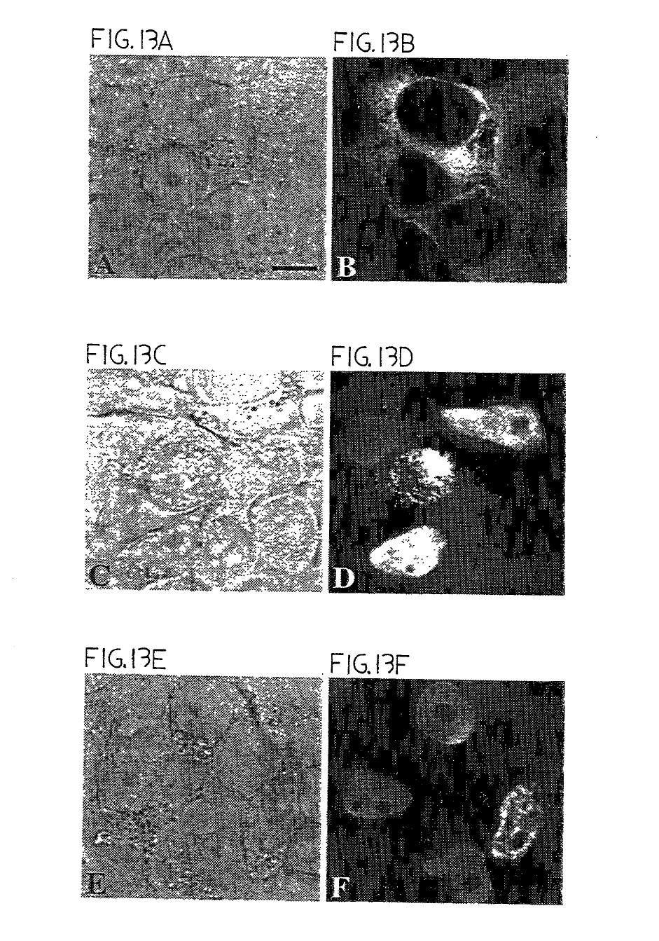

To determine whether the identified hLhx cDNAs represented a single genomic locus, Southern analyses were performed. Human Lhx3 cDNA probes hybridized to single DNA fragments (FIG. 12A), indicating that hLhx3 is encoded by a single gene. In Northern analyses, hLhx3 cDNA probes hybridized to a rare mRNA of approximately 2.4 kb in samples of adult human pituitary poly A⁺ RNA (FIG. 12B). Signals were not detected in samples of total pituitary RNA (FIG. 12B). As controls, RNA blots were also hybridized to a human Pit-1 cDNA probe (FIG. 12B).

In vitro transcription/translation was performed using rabbit reticulocyte lysates to generate radiolabeled hLhx3a and hLhx3b proteins from the identified cDNAs. Analysis of these proteins by electrophoresis revealed apparent molecular masses of approximately 60 kDa for both isoforms (FIG. 12C). As previously described elsewhere herein for porcine Lhx3 (see also Meier et al., 1999, Mol. Cell Endocrinol. 147:65-74), this apparent molecular mass is slightly larger than that predicted from the hLhx3a/b open reading frames, suggesting modification of the Lhx3 protein in these preparations or aberrant migration during electrophoresis due to composition.

To determine the intracellular localization of the hLhx3 isoforms, vectors for expression of hLhx3 isoforms as fusions with a multimerized green fluorescent protein (4xGFP) (Chatterjee et al., 1997, Exp. Cell Res. 236:346-350) were constructed. The 4xGFP protein localized exclusively to the cytoplasm of transfected cells (FIGS. 13A and 13B). By contrast, constructs containing hLhx3a or hLhx3b were detected in the nucleus (FIGS. 13C-F), indicating that they are nuclear proteins.

Expression of Lhx3 RNAs in the Pituitary

To precisely measure expression levels of hLhx3 gene transcripts in human pituitary samples, a “real time” quantitative PCR assay was developed as disclosed elsewhere herein. RNA was isolated from human pituitary tissue and cDNA was transcribed with a hLhx3-specific primer.

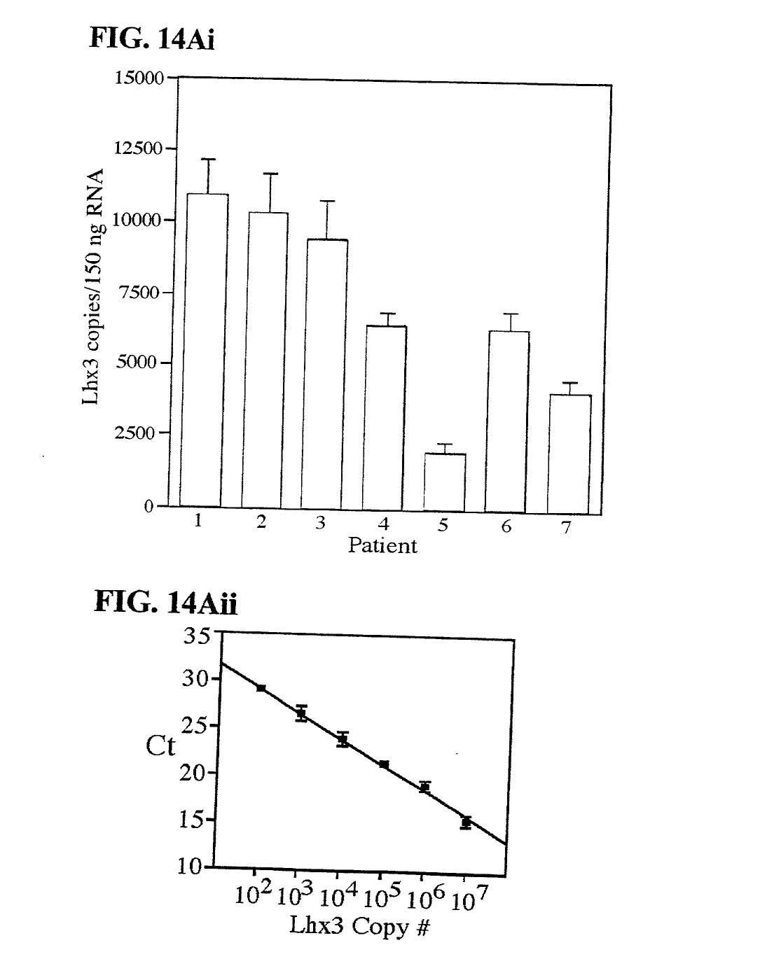

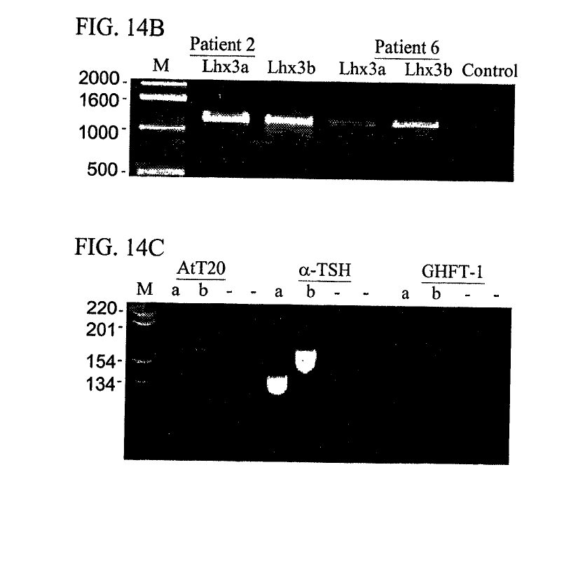

Quantitative PCR amplification reactions then were performed using three hLhx3-specific oligonucleotides: two to prime amplification of a region within the 3′-end of the mRNA and a fluorescent internal oligonucleotide that hybridized to PCR products to allow monitoring of the reaction. Serial dilutions of hLhx3 cDNA were amplified simultaneously with patient samples to generate a standard curve (FIG. 14A, inset). The standard curve was linear in a range from 100 to 10 million copies of hLhx3. Seven normal adult pituitary samples were assayed and hLhx3 was present at levels ranging from 2,000 to 11,000 copies/150 ng RNA with a mean value of 7,200 copies/150 ng RNA (FIG. 14A). This value is consistent with hLhx3 being a rare message.

In previous studies, it has been demonstrated that ubiquitously expressed genes such as P-actin are expressed at approximately 500,000 copies/150 ng RNA (Sloop et al., 1998, Biochem. Biophys. Res. Commun. 251:142-147). In specialized tissues such as adipose, mRNAs for hormones such as leptin are found at approximately 300,000 copies/150 ng RNA, and transcription factors such as CCAAT/enhancer binding protein-a (C/EBPα) are found at about 150,000 copies/150 ng RNA (Sloop et al., 1998, Biochem. Biophys. Res. Commun. 251:142-147).

To examine whether hLhx3 RNAs encoding the hLhx3a and hLhx3b isoforms are expressed in human pituitary samples, oligonucleotide primers were designed to specifically amplify either the hLhx3a or hLhx3b protein coding regions. RT-PCR experiments indicated that both forms are found in the adult pituitary (FIG. 14B).

To investigate whether the Lhx3 isoforms were expressed in specific cell types, expression of Lhx3 isoforms in established mouse pituitary cell lines was assessed. In the a-TSH thyrotrope tumor cell line (Akerblom et al., 1990, Mol. Endocrinol. 4:589-596), both mLhx3a and mLhx3b isoforms were readily detected (FIG. 14C). In atT20 corticotrope cells, low levels of mLhx3b were detected, and in some preparations, trace levels of mLhx3a were also observed. Neither isoform was detected in the GHFT-1 cell type. This Pit-1-expressing cell line represents an intermediate stage of pituitary development before differentiation and expression of trophic hormone genes (Lew et al., 1993, Genes Dev 7:683-693). These data are consistent with observations of the overall levels of Lhx3 gene expression by RNA analyses of these cell types (Seidah et al., 1994, DNA Cell Biol. 13:1163-1180; Bach et al., 1995, Proc. Natl. Acad. Sci USA 92:2720-2724; Zhadanov et al., 1995, Dev Dynam 202:354-364).

Differential Activation of Pituitary Hormone Genes by Human Lhx3 Isoforms

Lhx3 can activate anterior pituitary trophic hormone gene promoters (Bach et al., 1995, Proc. Natl. Acad. Sci. USA 92:2720-2724; Bach et al., 1997, Genes Dev. 11:1370-1380; Meier et al., 1999, Mol. Cell Endocrinol. 147:65-74). For example, the data disclosed previously elsewhere herein demonstrate that Lhx3 can induce transcription from the αGSU gene by specifically binding to a site located at −350 to −323 bp (known as the pituitary glycoprotein basal element, PGBE) within the proximal region of the promoter (Bach et al., 1995, Proc. Natl. Acad. Sci. USA 92:2720-2724; Meier et al., 1999, Mol. Cell Endocrinol. 147:65-74). This gene encodes the common subunit of the LH, FSH, and TSH anterior pituitary hormones. Other laboratories have shown that the Lhx2/LH-2 LIM homeodomain transcription factor also can recognize this element (Roberson et al., 1994, Mol. Cell. Biol. 14:2985-2993), and that this element is required to correctly restrict expression of the αGSU gene to pituitary gonadotropes and thyrotropes in transgenic mice (Brinkmeier et al., 1998, Mol. Endocrinol. 12:622-633).

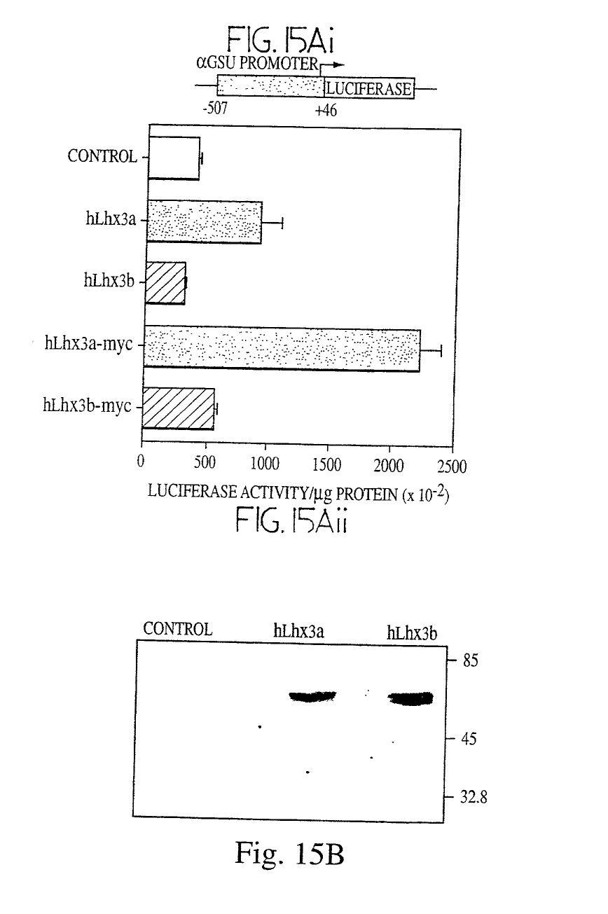

To test the ability of the hLhx3 isoforms to activate the αGSU gene, human embryonic 293 cells were transiently co-transfected with αGSU luciferase reporter genes and expression vectors containing full-length hLhx3a and hLhx3b cDNAs. 293 cells were used because they are of human origin, efficiently transfected, and do not express Lhx3. In the assays disclosed herein, hLhx3a activated the αGSU promoter (FIG. 15A). Surprisingly, hLhx3b did not induce transcription from this promoter (FIG. 15A). Similar experiments using expression vectors encoding hLhx3a or hLhx3b with carboxyl myc epitope tags gave the same results. That is, hLhx3a-myc activated the αGSU promoter and hLhx3b-myc was inactive (FIG. 15A). In these experiments, the epitope-tagged constructs were generally more active likely due to the fact that, in these constructs, the 3′-untranslated region of the cDNA is absent. The murine Lhx3 3′-untranslated region contains ATTTA sequence motifs that may confer instability to the RNA (Zhadanov et al., 1995, Genomics 27:27-32). Without wishing to be bound by any particular theory, these motifs are conserved in the human Lhx3 sequences, and the levels of both isoforms are therefore likely to be somewhat lower in the full-length cDNA experiments. An alternate explanation is that the single myc epitope confers an activation function to the Lhx3 molecules. This has been noted in experiments where multimers of five myc epitope tags are used (Ferreiro et al., 1998, Development 125:1347-1359). It is important to note, however, that the relative activities observed for the two Lhx3 isoforms are consistent for all types of expression constructs.

To confirm expression of the hLhx3 proteins in the transfected cells, Western blot analysis was performed using a specific anti-myc antibody. Both isoforms were detected at similar levels as protein species of approximately 60 kDa (FIG. 15B). This apparent molecular mass is similar to that of the in vitro translated hLhx3 proteins described above.

Western blots were quantified to determine relative expression levels of the hLhx3 isoforms. The observed expression levels of the two isoforms were similar. On average, the hLhx3b isoform was expressed at slightly higher levels (1.2-fold the level observed for hLhx3a).

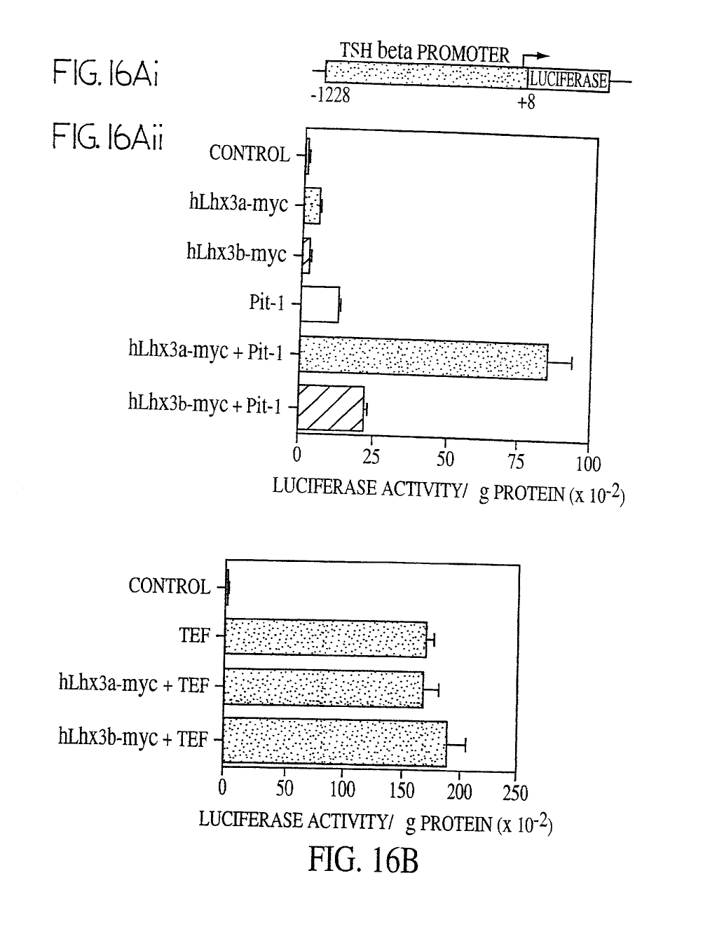

In addition to its ability to trans-activate the αGSU promoter, Lhx3 can induce pituitary trophic hormone gene promoters in synergy with pituitary transcription factors such as Pit-1 and Pitx1/P-Otx (Bach et al., 1995 Proc. Natl. Acad. Sci. USA 92:2720-2724; Bach et al., 1997, Genes Dev. 11:1370-1380; Meier et al., 1999, Mol Cell Endocrinol. 147:65-74). To examine whether the hLhx3 isoforms also had distinct abilities to cooperate with other factors in transcriptional induction, a TSHβ reporter gene was co-transfected with hLhx3a and hLhx3b expression vectors. Pit-1 and hLhx3a moderately activated the TSHβ promoter and together strongly induced transcription (FIG. 16A). Human Lhx3b only weakly activated the TSHβ promoter and did not effectively synergize with Pit-1 (FIG. 16A). As controls, similar experiments were performed with TEF. TEF is a PAR-bZIP transcription factor that strongly induces the TSHβ promoter (Drolet et al., 1991, Genes Dev. 5:1739-1753). The data disclosed herein demonstrate that TEF activated expression from the TSHβpromoter, and expression of hLhx3a or hLhx3b did not affect this activity (FIG. 16B), demonstrating that the observed synergy between hLhx3a and Pit-1 was specific.

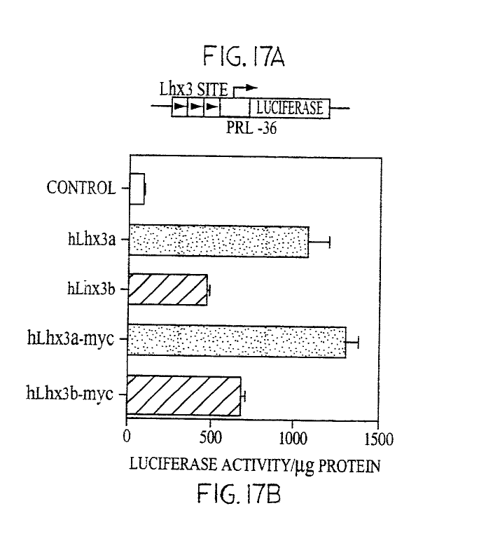

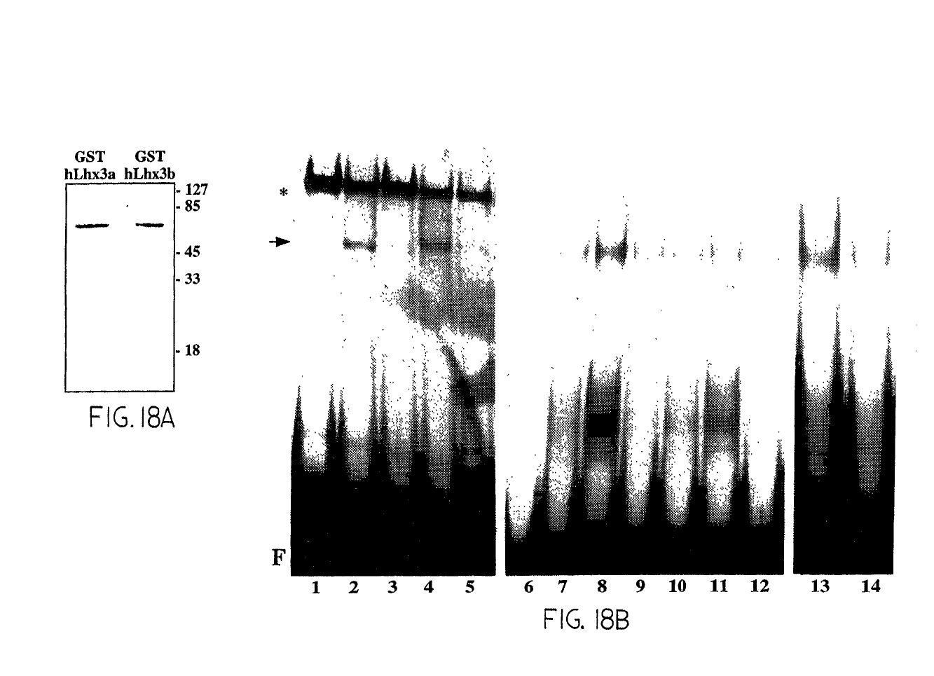

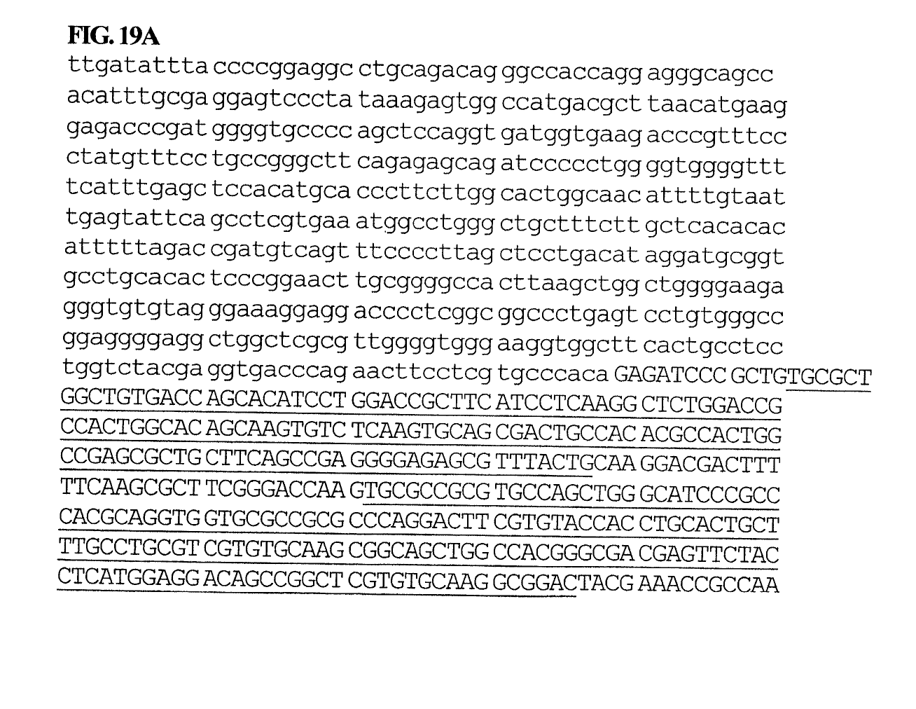

The αGSU and TSHβ promoters are complex regulatory structures containing recognition sites for multiple transcription factors. For example, the αGSU PHBE Lhx3 binding site appears to cooperate with other elements in gene regulation (Brinkmeier et al., 1998, Mol. Endocrinol. 12:622-633; Heckert et al., 1995, J. Biol. Chem. 270:26497-26504). Therefore, the abilities of hLhx3a and hLhx3b to activate a defined synthetic luciferase reporter gene containing three copies of a consensus Lhx3 DNA binding element cloned upstream of a minimal promoter were compared. Again, expression vectors for hLhx3a and hLhx3a-myc activated transcription from this reporter more effectively than the corresponding hLhx3b vectors (FIG. 17). These data further support the hypothesis that hLhx3a is a more potent activator of transcription than hLhx3b for target genes containing this type of recognition element.

DNA Binding Properties of hLhx3 Isoforms

The different abilities of hLhx3 isoforms to activate transcription from the tested reporter genes indicated that the hLhx3a- and hLhx3b-specific domains conferred distinct transcriptional properties upon the hLhx3 protein. The data show that the two isoforms were both expressed in the nucleus at similar levels (FIGS. 13 and 15). Without wishing to be bound by any particular theory, mechanisms for the different activities include the possibilities that the a-specific and b-specific domains are trans-activation or trans-repression domains, respectively, or that the a- and b-specific domains mediate interaction with distinct regulatory factors, or that the a- and b-specific domains modulate the DNA binding properties of hLhx3 differently. The LIM domains of Lhx3 repress DNA binding by the homeodomain (Bach et al., 1995, Proc. Natl. Acad. Sci USA 92:2720-2724; Meier et al, 1999, Mol. Cell Endocrinol 147:65-74). To test whether the a- and b-specific domains conferred distinct DNA binding properties upon the hLhx3 molecule, electrophoretic mobility shift assays (EMSAs) were performed to test binding to the αGSU −350/−323 bp Lhx3 site and to the Lhx3 consensus binding site. Experiments using in vitro translated native hLhx3 proteins disclosed herein demonstrate that hLhx3a binds to these sites more effectively than hLhx3b (FIG. 18B). Similar results were obtained with hLhx3 proteins containing carboxyl-terminal myc epitope tags (FIG. 18B).

To confirm this result, recombinant hLhx3 proteins were expressed in E. coli as fusions to the carboxyl terminus of glutathione-S-transferase (GST). This approach allowed the use of affinity-purified proteins of known concentration (FIG. 18A). EMSA reactions using the recombinant purified proteins again indicated that hLhx3a bound more effectively than hLhx3b to both DNA sites (FIG. 18B). Addition of excess unlabeled binding site DNA to hLhx3a EMSA reactions reduced the amount of bound complex, whereas it abolished the binding of hLhx3b (FIG. 17B). These experiments demonstrate that the b-specific domain inhibits the binding of hLhx3b to this class of DNA site. Further, the b-specific domain can perform this function when positioned at the amino terminus of the native protein or when placed in an internal context such as in the GST fusion proteins.

Through evolution, Lhx3-type proteins appear to have been largely conserved in structure, expression pattern, and function. The Lhx3/LIM3 gene is transiently expressed in the developing nervous systems in Drosophila, Xenopus, zebrafish, chickens, and mammals (Taira et al., 1993, Dev. Biol. 159:245-256; Seidah et al., 1994, DNA Cell Biol. 13:1163-1180; Bach et al., 1995, Proc. Natl. Acad. Sci. USA 92:2720-2724; Zhadanov et al., 1995, Dev. Dynam. 202:354-364; Glasgow et al., 1997, Dev. Biol. 192:405-419; Thor et al., 1999, Nature 397:76-80; Tsuchida et al., 1994, Cell 79:957-970), and it has been demonstrated to play important roles in specification of motor neuron subtypes in some of these species (Thor et al., 1999, Nature 397:76-80; Tsuchida et al., 1994, Cell 79:957-970; Sharma et al., 1998, Cell 95:817-828; Osumi et al., 1997, Development 124:2961-2972). In addition, Lhx3 is critical for both early development and terminal differentiation of the mammalian pituitary (Sheng et al., 1996, Science 272:1004-1007; Sheng et al., 1997, Science 278:1809-1812). Expression also is detected in other developing endocrine structures including the pineal gland (Taira et al., 1993, Dev. Biol. 159:245-256; Seidah et al., 1994, DNA Cell Biol. 13:1163-1180; Bach et al., 1995, Proc. Natl. Acad. Sci. USA 92:2720-2724; Zhadanov et al., 1995, Dev. Dynam. 202:354-364; Glasgow et al., 1997, Dev. Biol. 192:405-419) and the Drosophila ring gland (Thor et al., 1999, Nature 397:76-80), the major site of production of developmental hormones in higher dipterans. As summarized in FIG. 9, the LIM domains, the homeodomain, and the Lhx3/LIM3-specific domain are conserved in Lhx3-type proteins from Drosophila to humans.

The data disclosed herein demonstrate that proteins with distinct functional properties are generated from the human Lhx3 gene. During evolution, the increased complexity of organisms has required an extended number of regulatory factors with sufficient capacity to control the development of organ systems comprised of multiple, differentiated cell types. This increased molecular diversity has been accomplished by mechanisms such as the formation of additional genes by gene duplication; the generation of multiple proteins from single genes by the use of distinct promoters; and post-transcriptional processes. Post-transcriptional mechanisms include the use of alternate translation initiation codons, alternative RNA splicing, the production of protein isoforms from single RNAs by RNA editing, and proteolytic processing. For Lhx3-type proteins, it appears that both gene expansion (to produce genes encoding related proteins such as Lhx4) and RNA-mediated mechanisms generating isoforms such as hLhx3a and hLhx3b, have been used to provide a battery of proteins with important roles in the development of both neural and endocrine structures, including the anterior pituitary.

Alternate isoforms of LIM homeodomain proteins, non-homeodomain LIM proteins, and several other types of transcription factors are known. For example, the LIM homeodomain transcription factors, Lhx6.1a and Lhx6.1b, appear to be generated by an alternative splicing event that generates proteins with different carboxyl terminal amino acids (Kimura et al., 1999, J. Biochem. 126:180-187). These factors are expressed at different times in the developing mouse brain, but unique functional properties of the isoforms have not been demonstrated (Kimura et al., 1999, J. Biochem. 126:180-187). Alternate forms of LMO7 and LIM-kinase proteins lacking either the LIM domain or the dinase domain, respectively, have also been described (Putilina et al., 1998, Biochem. Biophys. Res. Commun. 252:433-439; Edwards and Gill, 1999, J. Biol. Chem. 274:11352-11361). The isoforms of these proteins have different expression patterns and distinct functional properties. Interestingly, Skn-1a and Skn-1i are alternatively spliced POU domain factors expressed in the epidermis that, like Lhx3, have different amino-terminal domains (Anderson et al., 1993, Science 260:78-82). Similar to the hLhx3b-specific domain, the 1-specific domain of Skn-1 inhibits DNA binding and gene trans-activation by this factor. However, primary amino acid sequence comparison between these inhibitory domains reveals no similarity.

Alternate isoforms with distinct functions and/or expression patters are also seen in the nuclear receptor protein superfamily. Examples include the distinct expression patterns of the isoforms of the peroxisome proliferator-activated receptor γ developmental regulatory protein (Tontonoz et al., 1994, Genes Dev. 8:1224-1234; Mukherjee et al., 1997, J. Biol. Chem. 272:8071-8076) and the unique transcriptional properties of the β2 thyroid receptor isoform (Langlois et al., 1997, J. Biol. Chem. 272:24927-24933).

In the mouse, isoforms of Lhx3 have been described (Zhadanov et al., 1995, Dev. Dynam. 202:354-364; Zhadanov 1995, Genomics 27:27-32). However, no difference in the function of the two mouse isoforms has been suggested or disclosed. Indeed, until the present invention, no LIM homeodomain proteins with alternate functional domains had ever been described. Without wishing to be bound by any particular theory, the hLhx3a- and hLhx3b-specific amino-terminal motifs may be novel functional domains derived throughout evolution to confer properties unique to Lhx3a and Lhx3b that allow specific roles in the development of the pituitary gland and neural structures.

Without wishing to be bound by any particular theory, comparison of the nucleic acid and amino acid sequences of the murine and human Lhx3 isoforms reveals, for the first time, that the human Lhx3b (Hb) clone disclosed herein is similar to murine Lhx3b (Mb), because the two amino-terminal domains share sequence homology even though the other portions of the sequences are less homologous (FIG. 9). In contrast, the human and murine Lhx3a amino-terminal domains share less, if any, sequence homology although some sequence features are similar (FIG. 9). Without wishing to be bound by theory, the data disclosed herein suggest that the Lhx3a forms are functionally related, but this function does not require conservation of the Lhx3a-specific domain.

The data disclosed herein demonstrate that hLhx3a is able to activate specific target genes, whereas hLhx3b is inactive and that these functional differences correlate with a reduced binding of hLhx3b to DNA elements within these genes (FIGS. 14-18). These data, and the observation that the Lhx3a-specific domain is poorly conserved in sequence and length (FIG. 9), together suggest that the Lhx3b-specific domain confers intramolecular inhibition of DNA binding and trans-activation functions. Data disclosed previously elsewhere herein demonstrate that the LIM domains of mammalian Lhx3 proteins inhibit DNA binding (see also Bach et al., 1995, Proc. Natl. Acad. Sci. USA 92:2720-2724; Meier et al., Mol. Cell. Endocrinol. 147:65-74), and others have made similar observations for several LIM homeodomain proteins (reviewed in Curtiss and Heilig, 1998, Bioessays 20:58-69). Therefore, without wishing to be bound by any particular theory, the Lhx3b-specific domain may inhibit DNA binding by configuring the LIM domains such that they exert a greater inhibitory function upon the homeodomain. Alternatively, the Lhx3specific domain may function independently of the LIM domains, or it may exert both LIM-dependent and independent effects.

The data disclosed herein suggest that the alternate hLhx3 isoforms have distinct functions within the developing pituitary gland and neural structures such as the embryonic spinal cord. The results disclosed herein further suggest that the described Lhx3 isoforms are critical to thyrotrope differentiation and maintenance because both are expressed in the a-TSH cell line, and hLhx3a activated the αGSU and TSHβ genes. The low level of Lhx3 expression in AtT20 cells is consistent with observation of Lhx3 knockout mice that have some corticotropes, but lack other pituitary cell types (Sheng et al., 1996, Science 272:1004-1007). Without wishing to be bound by any particular theory, it may be that the more abundant Lhx3b isoform in AtT20 cells plays a unique role in differentiation of this cell type.

The described hLhx3 isoforms may have distinct target genes or, dependent on its expression profile, hLhx3b may play a direct or indirect dominant negative role. Experiments in the mouse have suggested that in this species Lhx3 a may be expressed earlier than Lhx3b during development (Zhadanov et al. 1995, Genomics 27:27-32). Specification of differentiated pituitary cell phenotypes appears to be controlled by the combinatorial actions of multiple tissue-specific transcription factors and signaling proteins (reviewed in Watkins-Chow and Camper, 1998, Trends Genet. 14:284-290; Kioussi et al., 1999, Mech. Dev. 81:23-35; Sheng et al., 1999, Trends Genet. 15:236-240). The differential activities or expression patterns of alternate forms of essential members of this program, such as Lhx3 and Pit-1, may play critical roles in the determination of pituitary cell fates. Indeed, isoforms of Pit-1 that have distinct properties and expression patterns have been described (e.g., Haugen et al., 1997, J. Biol. Chem. 268:20818-20824; Diamond et al., 1999, Mol. Endocrinol. 13:228-238; and reviewed in Rhodes et al., 1996, J. Anim. Sci. 74:94-106).

In addition, the described Lhx3 isoforms may interact differently with broadly expressed regulatory proteins or transcriptional co-activators/co-repressors. For example, a conserved family of nuclear LIM domain-interacting proteins, known as NL1/Ldb1 /CLIM/Chip, has been characterized (Jurata and Gill, 1997 Mol. Cell Biol. 17:5688-5698; Jurata et al., 1998, J. Biol. Chem. 273:3152-3157; Breen et al., 1998, J. Biol. Chem. 273:4712-4717). These proteins appear to be regulatory partners for LIM homeodomain factors and can mediate homo-and heterodimerization of these factors. Recently, a RING-H2 zinc finger protein, RLIM, was identified and also described as a regulatory partner for LIM homeodomain factors (Bach et al., 1999, Nature Genet. 22:394-399). This c-regulatory protein appears to recruit the Sin3a/histone deacetylase co-repressor complex to function as a LIM-associated inhibitory factor (Bach et al., 1999, Nature Genet. 22:394-399). Co-regulators such as NLI proteins and RLIM may differentially modulate the ability of Lhx3 isoforms to regulate target genes.

The detection of hLhx3 in the pituitary gland and the demonstration of its ability to regulate pituitary trophic hormone gene promoters suggest a continued role for Lhx3 in the adult human pituitary gland in maintenance of hormone gene expression. This study also provides tools for future investigations examining the role of hLhx3 in pituitary diseases such as combined pituitary hormone deficiency (CPHD) and pituitary tumor disease. Further studies of hLhx3 and related factors will extend our understanding of the developmental program that establishes the hormone-releasing cells of the human pituitary gland and will facilitate future protocols designed to treat pituitary diseases, disorders, or conditions.

### EXAMPLE 5

### Structure of the Human Lhx3 Neuroendocrine Transcription Factor Gene and Mapping to the Subtelomeric Region of Chromosome 9

The experiments presented in this example may be summarized as follows.

Lhx3/LIM3/P-LIM is a LIM homeodomain transcription factor required for motor neuron specification and organogenesis of the pituitary gland. The LIM homeodomain (HD) proteins constitute a subfamily of HD transcription factors that regulate many aspects of mammalian development. They contain two cysteine-rich zinc finger-like LIM motifs that mediate protein-protein interactions with other transcription factors and nuclear co-activator/co-repressor proteins (Bach et al., 1999, Nature Genetics 22:394-399; Meier et al., 1999; Mol. Cell. Endocrinol. 147:65-74). In addition, regulatory factors in this class possess a characteristic DNA-binding HD. Cross-species comparison of Lhx3 protein sequences reveals conservation of the LIM and HD domains and of a motif in the carboxyl-terminus known as the Lhx3/LIM3-specific domain (Glasgow et al., 1997, Dev. Biol. 192:405-419; Thor et al., 1999, Nature 397:76-80; Meier et al., 1999, Mol. Cell. Endocrinol. 147:65-74; and data disclosed previously elsewhere herein).

Lhx3 is transiently expressed in the developing nervous system, then it is detected in the primordial structure known as Rathke's pouch, and expression persists in the pituitary of adults (Seidah et al., 1994, DNA Cell Biol. 13:1163-1180; Bach et al., 1995, Proc. Natl. Acad. Sci. USA 92:2720-2724; Zhadanov et al., 1995, Dev. Dynamics 202:354-364; Meier et al., 1999; Mol. Cell. Endocrinol. 147:65-74; Sloop et al., 1999, Mol. Endocrinol. In press; and data disclosed herein). Lhx3 null mutant mice die soon after birth, lack the anterior and intermediate lobes of the pituitary gland (Sheng et al., 1996, Science 272:1004-1007), and have defects in motor neuron specification (Sharma et al., 1998, Cell 95:817-828).

Lhx3 is required for the differentiation of the pituitary somatotrope, gonadotrope, lactotrope, and thyrotrope cell lineages (Sheng et al., 1996, Science 272:1004-1007). These cell types secrete hormones that regulate physiological functions including growth, reproduction, lactation, and metabolic homeostasis.

In mammals, two Lhx3 protein isoforms (Lhx3a and Lhx3b) exist that share common LIM and DNA-binding domains but which possess distinct amino terminal sequences (Zhadanov et al., 1995, Dev. Dynamics 202:354-364; Sloop et al., 1999, Mol. Endocrinol. In press; data disclosed herein). The data disclosed previously elsewhere herein demonstrate the different abilities of human LHX3a and LHX3b to activate pituitary trophic hormone genes such as the alpha-glycoprotein and thyroid-stimulating hormone-beta genes. The LHX3a isoform binds to DNA elements within the regulatory regions of these genes and can activate gene expression, acting alone or with other pituitary factors. By contrast, LHX3b binds with lower affinity to these DNA binding sites and either is inactive or only weakly capable of gene activation. The data disclosed herein demonstrate the genomic structure and chromosomal localization of the human LHX3 gene.

The Materials and Methods used and the Results of the experiments presented in this example are now described.

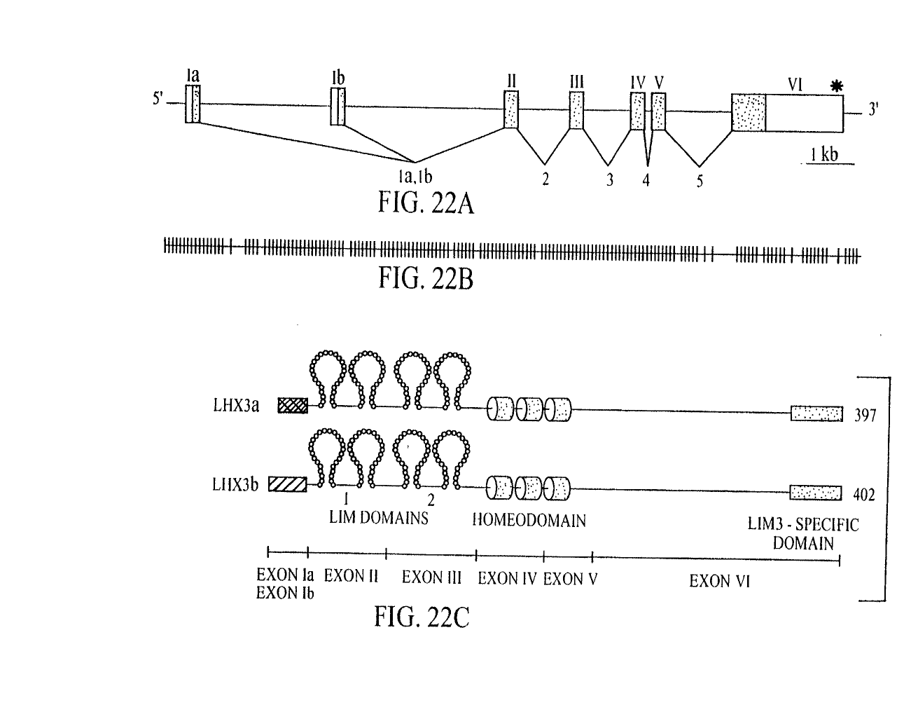

Potential exon-intron boundaries of human LHX3 were predicted by alignment of human LHX3 cDNA sequences disclosed previously elsewhere herein with the mouse Lhx3 gene (Zhadanov et al., 1995, Genomics 27:27-32). PCR primers were designed near potential exon-intron boundaries and were used to amplify each human LHX3 intron from the genomic DNA of normal adults. Intron DNA sequences were aligned with LHX3 cDNA sequences to allow analysis of splice site junctions. Each exon-intron boundary conforms to the GT/AG splice donor/splice acceptor consensus (Table 1). The human LHX3 gene contains seven coding exons and six introns, and the locus is 66% GC-rich and CpG dinucleotide-rich in composition (FIGS. 22A and 22B).

TABLE I

| Exon | | Sequence at Intron-Exon Boundary | | Intron | |
|---|---|---|---|---|---|
| | Size | | | | Size |
| Identity | (bp) | Splice Acceptor | Splice Donor | Identity | (bp) |
| Ia | ≧18 | 5′UTR..ccgcgATGCTG | ACTCGGGgtaag | 1a | 1775 |
| | 3 | M L | T R | | |
| Ib | ≧21 | 5′UTR..gacccATGGAG | CGCCGAGgtggg | 1b | 2191 |
| | 3 | M E | | | |
| II | 172 | cacagAGATCCCG | TTTTTCAAgtgag | 2 | 701 |
| | | E I P | F F | | |
| III | 203 | cgcagGCGCTTC | CAGCGAGgtcag | 3 | 617 |
| | | K R F | Q R | | |
| IV | 152 | cgcagAGGCCGAG | GTGCAGgtcag | 4 | 87 |
| | | E A E | V Q | | |
| V | 168 | cccagGTTTGGTTC | TTCCCCGgtagg | 5 | 909 |
| | | V W F | | | |
| VI | +419 | tgcagATGAGCCT | CAGTTCtgacc..3′UTR | | |
| | | D E P | Q F * | | |

Similar to other proteins in this class of transcription factors, the coding regions for the functional protein domains of LHX3 are contained within individual exons (FIG. 22C). Exons 1a and 1b code for the amino termini of the alternate LHX3 isoforms. Exons II and III code for the LIM1 and LIM2 domains, respectively. The DNA-binding HD is encoded by exons IV and V. These exons are separated by an 87 bp intron (intron 4). The Lhx3/LIM-specific domain is contained within exon VI. By comparison to the mouse gene (Zhadanov et al., 1995, Genomics 27:27-32), the genomic organization of human LHX3 appears to be conserved in mammals. A similar gene structure is present in the Drosophila and zebrafish Lhx3/LIM3 genes (Glasgow et al., 1997, Dev. Biol. 192:405-419; Thor et al., 1999, Nature 397:76-80). with the important difference that these genes appear to produce only one form of the protein.

The gene structure of LHX3 is similar that of other LIM homeodomain factors such as the human LHX1 gene (Singh et al., 1991, Proc. Natl. Acad. Sci. USA 88:10706-10710). By contrast, the first LIM domain of mouse Lhx8 is coded by two exons and the HD exon contains two introns (Kitanaka et al., 1998, Genomics 49:307-309). The homeobox intron is not present in the Isl-1 LIM HD factor genes (reviewed in Dawid et al., 1995, C. R. Acad. Sci. III 318:295-306). It is likely that the location of protein domains within individual exons has allowed exon duplication and shuffling processes and higher order gene duplication events to occur in the generation of multiple LIM and HD encoding genes.

The human LHX3 gene also shares regions of similarity with the mouse Lhx3 gene outside of protein coding regions, suggesting critical functional roles for these DNA elements. For example, the 3′ end of intron la contains the presumed LHX3b promoter. This sequence is a highly conserved, CpG-rich region (FIG. 22B) that contains 3 putative Sp-1 transcription factor binding sites located -195, -175, -159 bp relative to the start of the LHX3b coding sequence. These sites are conserved in the mouse Lhx3 gene (Zhadanov et al., 1995, Genomics 27:27-32), supporting the suggestion that the LHX3 isoforms are generated from separate promoters.

Further comparison of this region fails to locate TATA-box or initiator elements in either species. Many ubiquitously expressed genes contain TATA-less promoters that are CpG dinucleotide-rich in their promoter regions. By contrast, Lhx3 expression is highly restricted to the pituitary gland after being transiently expressed in the developing nervous system. Assessment of the level of Lhx3a and Lhx3b expression in established rodent pituitary cell lines as disclosed previously elsewhere herein is consistent with the hypothesis that Lhx3a and Lhx3b promoters are subject to differential cell-specific regulation.

Another conserved element is an ATTTA motif located 1016 bp downstream of the LHX3 stop codon (FIG. 22A). This element has been previously shown to be present in genes that produce mRNAs with short half-lives (Sachs, 1993, Cell 74:413-421). Indeed, as demonstrated by the data disclosed previously elsewhere herein, assaying the function of LHX3 cDNAs demonstrated increased capacity to activate target genes when this region is removed.

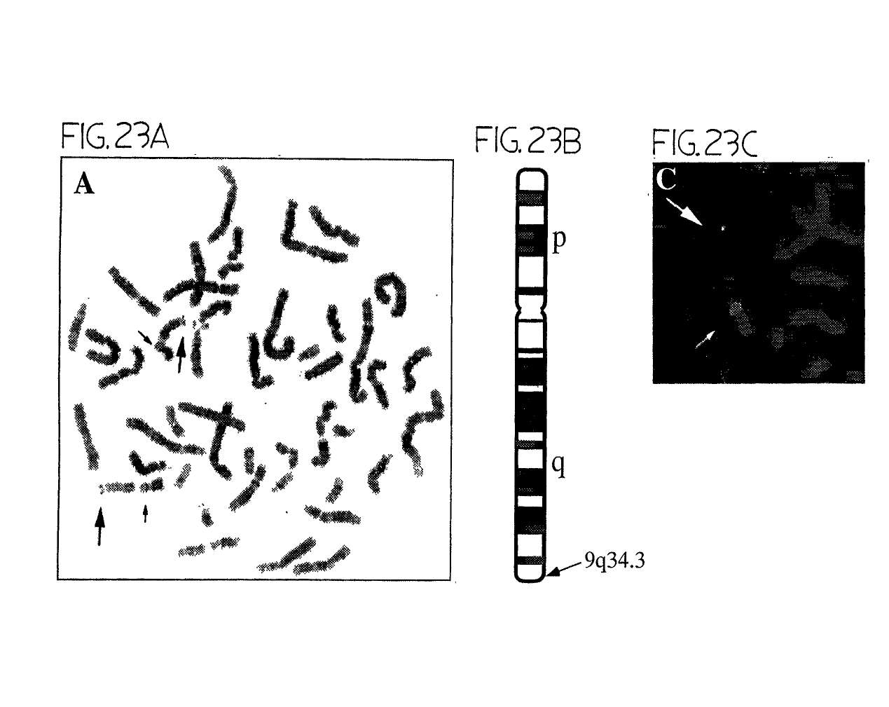

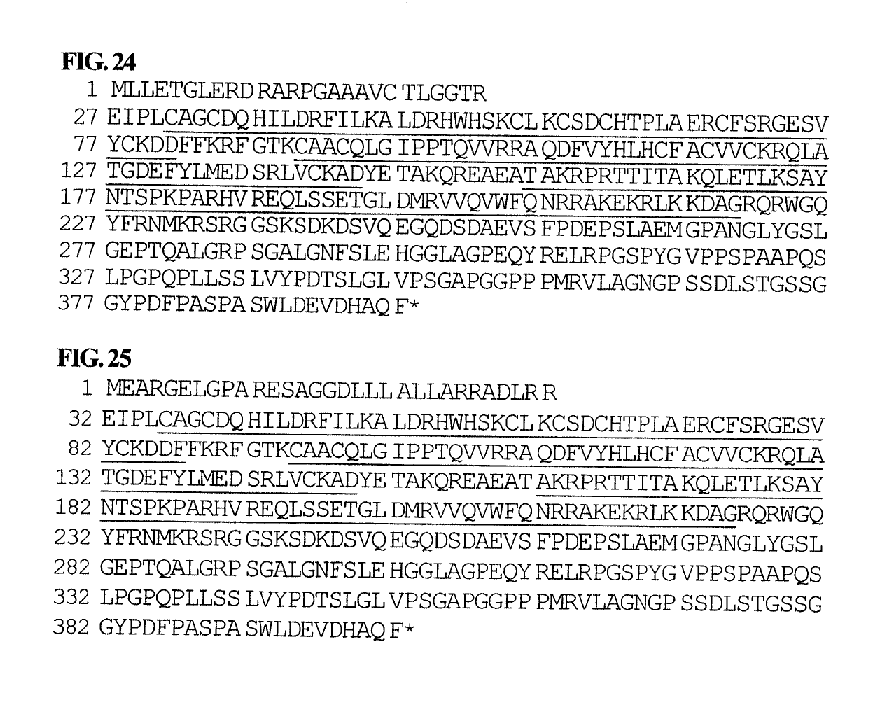

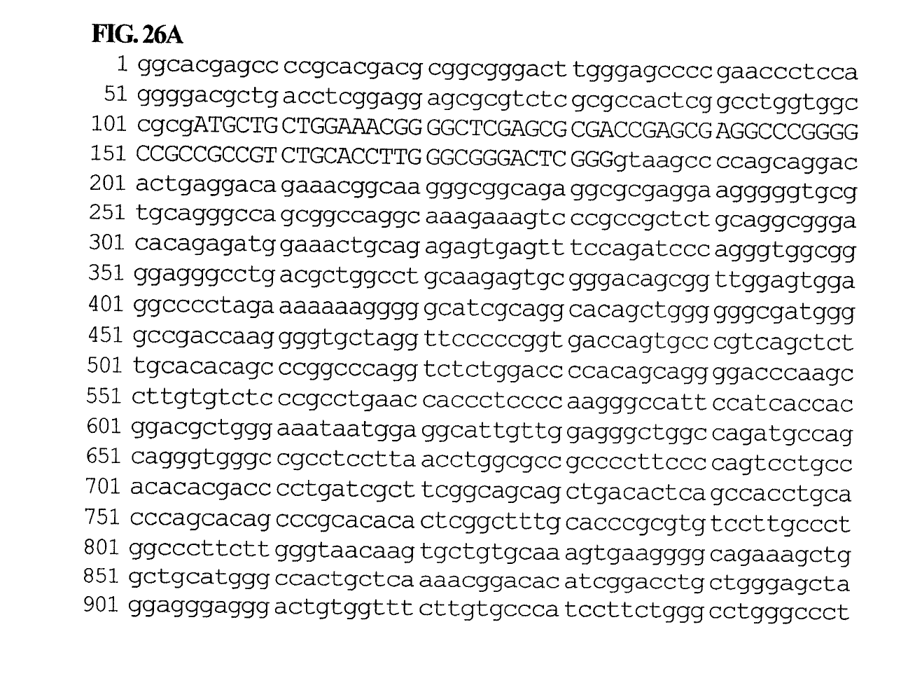

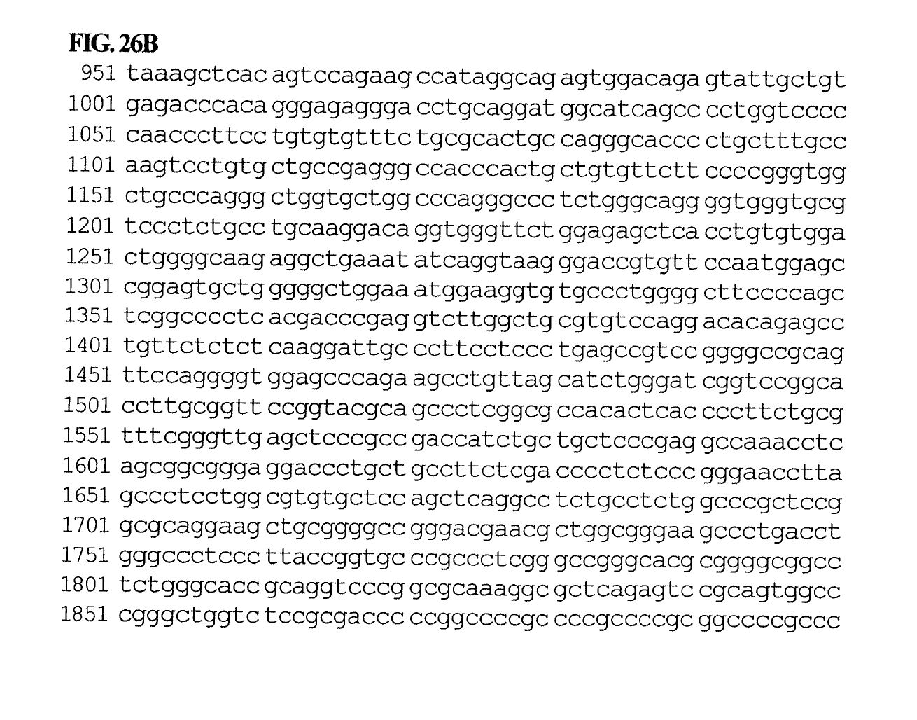

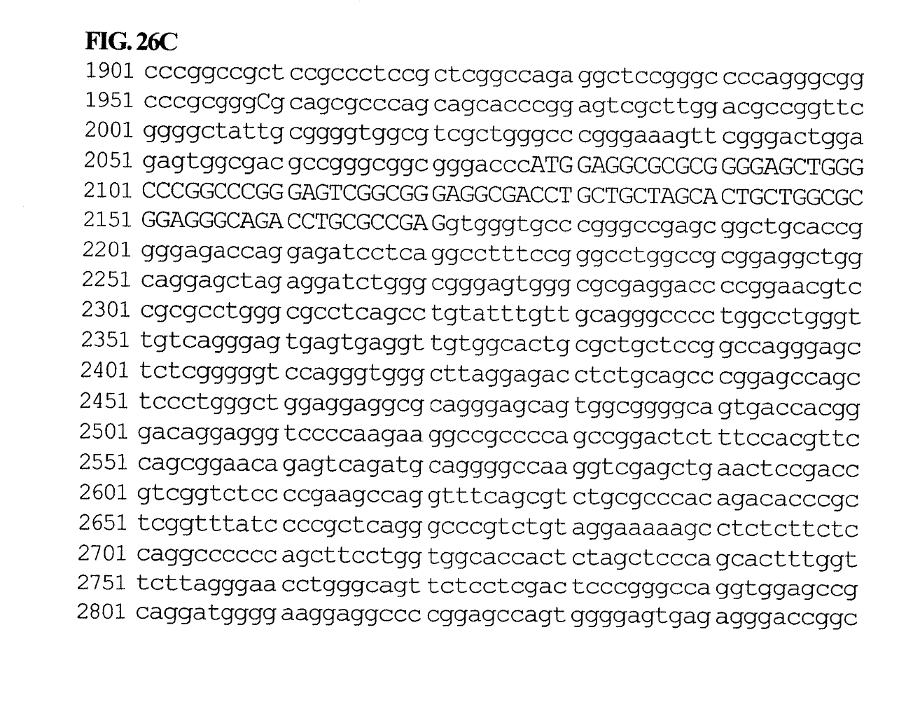

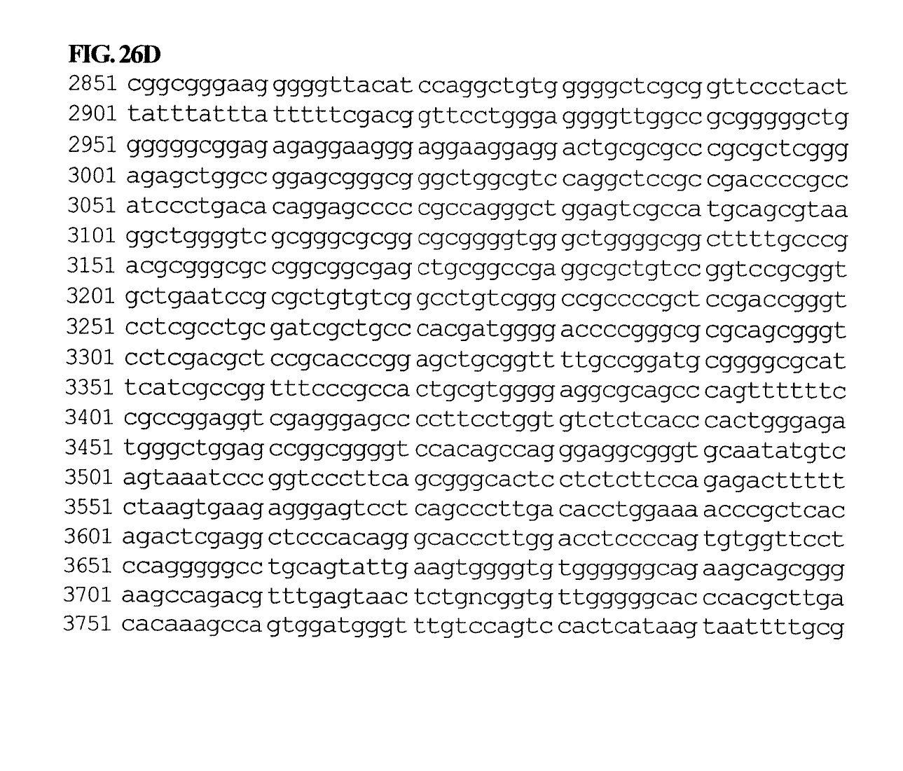

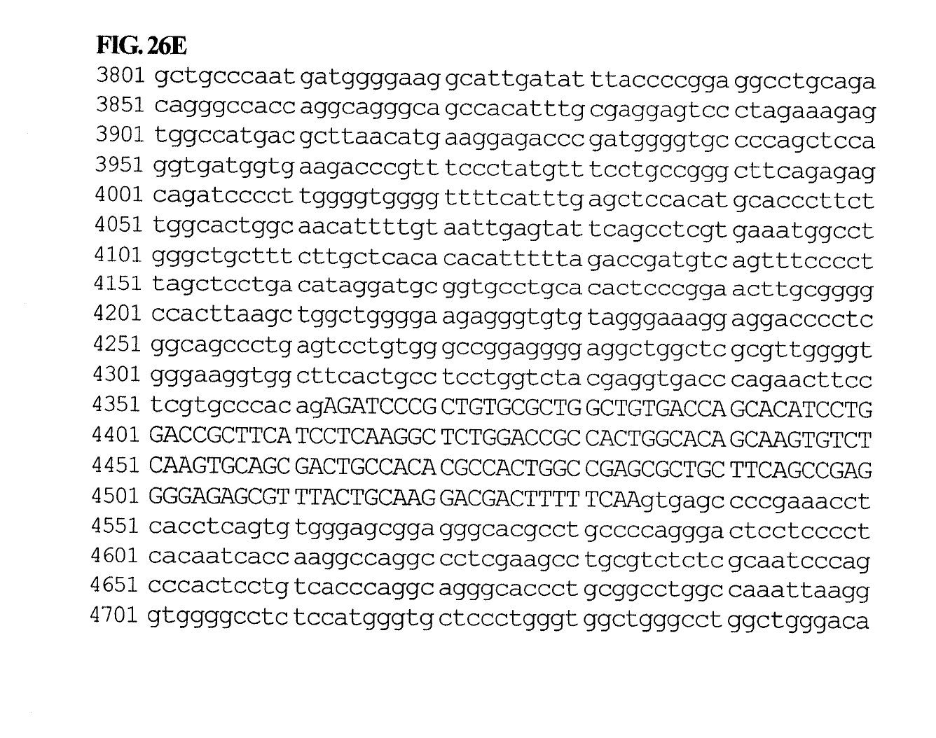

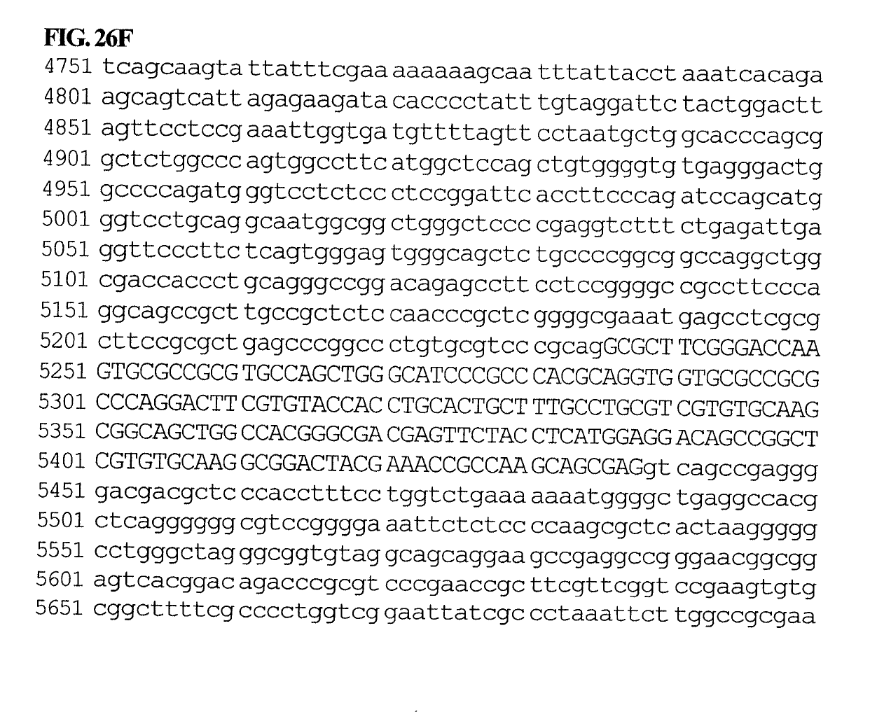

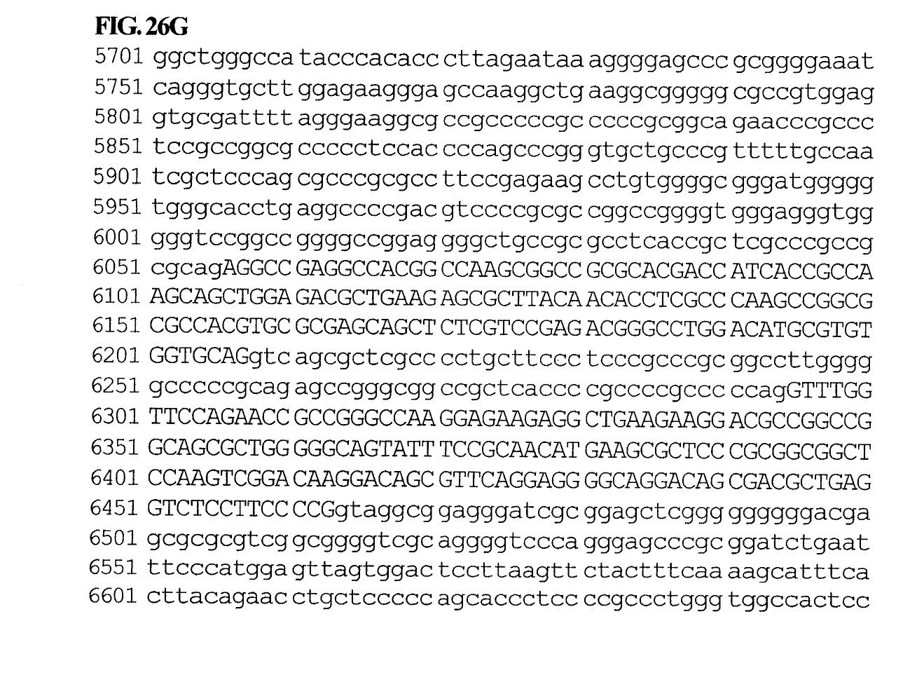

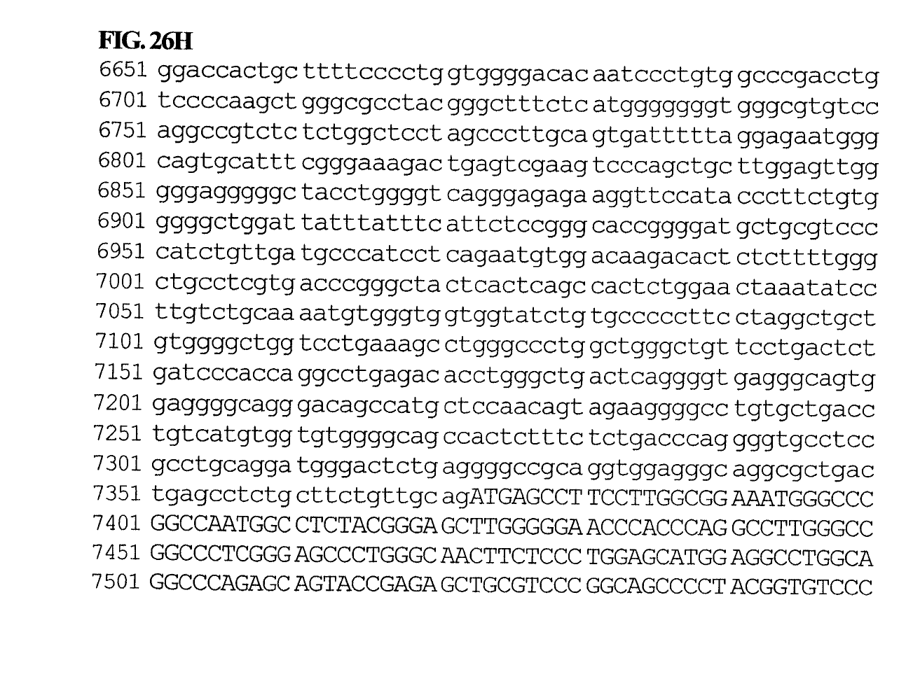

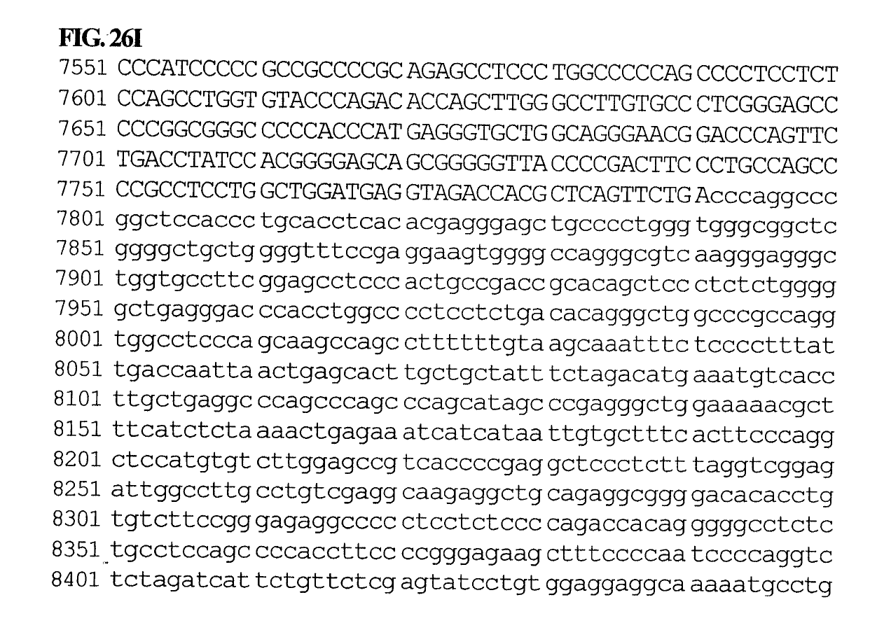

The mouse Lhx3 gene has been mapped to Chromosome (Chr) 2 near the Notch1 locus (Zhadanov et al., 1995, Genomics 27:27-32; Mbikay et al. 1995, Mamm. Genome 6:818-819). The data disclosed herein demonstrate mapping of the human LHX3 gene to band 9q3⁴0.3 using fluorescence in situ hybridization (FISH) (FIGS. 23A and 23B). This region also includes the human Notch homologue, TAN-1, and the retinoid X receptor alpha genes. A signal was clearly observed on both homologous Chr 9s in all metaphase spreads examined. The identity of Chr 9 was confirmed by co-hybridization with a probe specific for the Chr 9 centromere (FIG. 23A).

The 9q34.3 band is the most distal region of Chr 9 and includes the telomeric/subtelomeric region. Hybridization experiments also were performed using LHX3 probes in tandem with a 9q specific subtelomeric region probe that maps within 300 kb of the telomere. The signals for the two probes could not be clearly separated, as indicated by the overlapping probe colors (FIG. 23C), demonstrating that LHX3 is located close to the telomeric region.

It has been demonstrated that the subtelomeric regions of chromosomes are often high in GC and CpG dinucleotide content (Flint et al., 1997, Nature Genet. 15:252-257), which is in accord with the LHX3 gene composition (FIG. 22B). Mutations in the Pit-1 (POU1F1) and Prophet of Pit-1 (PROP1) pituitary transcription factor genes cause combined pituitary hormone deficiency (reviewed in Cohen et al., 1999, Trends End. Metab. 10:326-332). Although, to date, no relevant human disease has been linked to the 9q34.3 locus, and without wishing to be bound by theory, it may be that pituitary or nervous system disorders may result from mutations in LHX3. Whereas targeted ablation of the entire Lhx3 gene is lethal in mice (Sheng et al., 1996, Science 272:1004-1007), less severe phenotypes may result from mutations in the functional domains of LHX3, particularly mutations in the alternate LHX3“a” or “b” domains. Indeed, loss of function experiments targeting different isoforms of the Drosophila Prickle LIM protein result in phenotypes dissimilar to that of the complete null mutant (Gubb et al., 1999, Genes Dev. 13:2315-2327). Identification and chromosomal localization of LHX3 will facilitate future genetic evaluation of humans with pituitary and neuronal developmental disorders that may be caused by a loss of function of this factor.

Sequences disclosed herein have been submitted in the following GenBank entries: AF1566888 (hLhx3a), AF156889 (hLhx3b), AF188738 (hLhx3 Intron 1a), AF188739 (hLhx3 Intron 1b), AF188740 (hLhx3 Intron 2), AF188741 (hLhx3 Intron 3), AF 188742 (hLhx3 Intron 4), and AF 188743 (hLhx3 Intron 5).

### EXAMPLE 6

### Diagnostic Procedures to Detect Mutations in Lhx3

Mutations in LHX3 may cause congenital pituitary and/or central nervous system diseases/disorders in humans. Disclosed herein is a description materials and methodology of LHX3 gene mutation diagnostic assay.

Human genomic DNA is extracted from peripheral blood by standard procedures. Human LHX3 exons are amplified by the polymerase chain reaction (PCR) using primers specific to LHX3 intron DNA sequences as disclosed previously elsewhere herein. Each PCR reaction contains 2.5 U Expand High Fidelity DNA polymerase mixture (Roche Molecular Biochemicals, Indianapolis, Ind.), 10 mM dATP, dCTP, dGTP, and dTTP, 200 ng human genomic DNA, and 10 pmols of each forward and reverse primer. PCR reactions for each exon utilize unique buffer conditions. PCR buffers (MasterAmp 2× PCR PreMix D,G,H,I buffers) are from Epicentre Technologies (Madison, Wis.).

Exon 1a (Ia) was amplified using 5′-tgacctcggaggagcgcgtct-3′ (SEQ ID NO:46) and 5′-caaccgctgtcccgcactctt-3′ (SEQ ID NO:103) using MasterAmp 2X PCR PreMix H buffer and the above reaction components.

Exon 1b (Ib) was amplified using 5′-gaaagttcgggactggagagt-3′(SEQ ID NO:104) and 5′-cagtgccacaacctcactca-3′ (SEQ ID NO:105) with Master Amp 2X PCR PreMix G buffer and the above reaction components.

Exon 2 (II) was amplified using 5′-tacgaggtgacccagaactt-3′ (SEQ ID NO:106) and 5′-cctggccttggtgattgtga-3′ (SEQ ID NO:107) with MasterAmp 2× PCR PreMix G buffer and the above reaction components.

Exon 3 (III) was amplified using 5′-tttcagaccaggaaaggtgg-3′ (SEQ ID NO:108) and 5′-cgaaatgagcctcgcgcttc-3′ (SEQ ID NO:109) with MasterAmp 2× PCR PreMix I buffer and the above reaction components.

Exons 4 (IV) and 5(V) were amplified using 5′-gctgccgcgcctcaccgct-3′ (SEQ ID NO:110) and 5′-aggagtccactaactccatg-3′ (SEQ ID NO:111) with MasterAmp 2× PCR PreMix D buffer and the above reaction components.

Exon 6 (VI) is amplified using 5′-cgctgactgagcctctgctt-3′ (SEQ ID NO:112) and 5′-cctcgtgtgaggtgcagggt-3′(SEQ ID NO:113) with MasterAmp 2× PCR PreMix D buffer and the above reaction components.

For the amplification reactions above, PCR cycling parameters are as follows: 94° C. for 2 minutes, 94° C. for 10 seconds, 66° C. for 10seconds, 72° C. for 1 minute for two cycles, 95° C. for 10 seconds, 64.5° C. for 10 seconds, 72° C. for 1 minute for 2 cycles, 96° C. for 10 seconds, 63° C. for 10 seconds, 72° C. for 1 minute for 2 cycles, 96° C. for 10 seconds, 61.5° C. for 10 seconds, 72° C. for 1 minute for 2 cycles, 96° C. for 10 seconds, 60° C. for 10 seconds, 72° C. for 1 minute for 20 cycles. The PCR reaction products are analyzed on 1% agarose, Tris-borate gels. The PCR products are ligated into pCRII-TOPO (Invitrogen, Carlsbad, Calif.) and sequenced on both strands by automated DNA sequencing using a Perkin Elmer DNA Sequencer as described previously elsewhere herein.

The fill length sequence of human Lhx3 is set forth in Figure XX (SEQ ID NO:22).

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated nucleic acid encoding a mammalian Lhx3, wherein said nucleic acid is selected from the group consisting of an isolated nucleic having at least about 88% identity with at least one of SEQ ID NO:1, SEQ ID NO:13, and SEQ ID NO:15, and further wherein said isolated nucleic acid has at least about 88.5% identify with at least one of SEQ ID NO:7, SEQ ID NO:9, and SEQ ID NO:11.

2. The isolated nucleic acid of claim 1, wherein the nucleic acid sequence of said nucleic acid is SEQ ID NO:1.

3. An isolated nucleic acid encoding a porcine Lhx3, said nucleic acid having at least about 88% identity with a nucleic acid selected from the group consisting of SEQ ID NO:1, SEQ ID NO:13, and SEQ ID NO:15.

4. An isolated nucleic acid encoding a human Lhx3, said nucleic acid having at least about 88.5% identity with a nucleic acid having the nucleotide sequence of at least one of SEQ ID NO:7, SEQ ID NO:9, and SEQ ID NO:11.

5. The isolated nucleic acid of claim 1, said nucleic acid further comprising a nucleic acid encoding a tag polypeptide covalently linked thereto.

6. The isolated nucleic acid of claim 5 wherein said tag polypeptide is selected from the group consisting of a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, and a maltose binding protein tag polypeptide.

7. The isolated nucleic acid of claim 1, said nucleic acid further comprising a nucleic acid encoding a promoter/regulatory sequence operably linked thereto.

8. A vector comprising the nucleic acid of claim 1.

9. A recombinant cell comprising the isolated nucleic acid of claim 1.

10. An isolated polypeptide encoded by the nucleic acid of claim 1.

11. The isolated polypeptide of claim 1, wherein said polypeptide shares at least about 93% identity with a polypeptide having the amino acid sequence SEQ ID NO:2.

12. The isolated polypeptide of claim 1, wherein said polypeptide shares at least about 94.5% identity with a polypeptide having the amino acid sequence of at least one of SEQ ID NO:8, SEQ ID NO:10, and SEQ ID NO:12.

13. An antibody that specifically binds with the isolated polypeptide of claim 10, or a fragment thereof.

14. The antibody of claim 13, wherein said antibody specifically binds with a portion of said isolated polypeptide selected from the group consisting of a portion from about amino acid residue 1 to about amino acid residue 26 of SEQ ID NO:10, a portion from about amino acid residue 1 to about amino acid residue 31 of SEQ ID NO:12, a portion from about amino acid residue 1 to about amino acid residue 29 of SEQ ID NO:14, and a portion from about amino acid residue 1 to about amino acid residue 31 of SEQ ID NO:16.

15. The antibody of claim 13, wherein said antibody is selected from the group consisting of a polyclonal antibody, a monoclonal antibody, and a synthetic antibody.

16. A composition comprising the isolated nucleic acid of claim 1 and a pharmaceutically-acceptable carrier.

17. A composition comprising the isolated polypeptide of claim 10 and a pharmaceutically-acceptable carrier.

18. An isolated nucleic acid encoding a mammalian Lhx3, wherein said nucleic acid specifically hybridizes under high stringency conditions to a nucleic acid selected from the group consisting of a nucleic acid having the nucleic acid sequence SEQ ID NO:1, a nucleic acid having the nucleic acid sequence SEQ ID NO:7, a nucleic acid having the nucleic acid sequence SEQ ID NO:9, a nucleic acid having the nucleic acid sequence SEQ ID NO:11, a nucleic acid having the nucleic acid sequence SEQ ID NO:13, and a nucleic acid having the nucleic acid sequence SEQ ID NO:15, or a nucleic acid complementary to any one of SEQ ID NO:1, SEQ ID NO:7, SEQ ID NO:9, SEQ ID NO:11, SEQ ID NO:13, and SEQ ID NO:15.

19. An isolated nucleic acid having at least about 45% identity with from about nucleotide −104 to about nucleotide 78 of SEQ ID NO:9.

20. An isolated nucleic acid having at least about 67% identity with from about nucleotide −119 to about nucleotide 93 of SEQ ID NO:11.

21. A method for detecting the presence or absence of a nucleic acid encoding a mammalian Lhx3 having at least about 88.5% homology with a nucleic acid having the sequence SEQ ID NO:7 in a sample, said method comprising
(a) contacting a sample with a nucleic acid probe or primer which specifically hybridizes with said nucleic acid; and
(b) determining whether said nucleic acid probe or primer binds with a nucleic acid in said sample whereby when said nucleic acid probe or primer binds with a nucleic acid in said sample, said sample contains a nucleic acid which specifically hybridizes with said nucleic acid,
thereby detecting the presence or absence of said nucleic acid in a sample.

22. The method of claim 21, wherein said sample comprises mRNA molecules.

23. A method for detecting the presence or absence of a nucleic acid encoding a mammalian Lhx3 in a sample, said method comprising
(a) amplifying a nucleic acid encoding a mammalian Lhx3 present in a sample wherein said nucleic acid shares about 88.5% identity with a nucleic acid sequence selected from the group consisting of SEQ ID NO:1, SEQ ID NO:7, SEQ ID NO:9, SEQ ID NO:11, SEQ ID NO:13, and SEQ ID NO:15;
(b) detecting any amplified target nucleic acid formed in (a) whereby when said nucleic acid is amplified said sample contains said nucleic acid which encodes a mammalian Lhx3,
thereby detecting the presence or absence of a nucleic acid encoding a mammalian Lhx3 in a sample.

24. A method of quantitatively detecting a nucleic acid encoding a mammalian Lhx3 in a sample, said method comprising
(a) contacting a target nucleic acid encoding a mammalian Lhx3 present in a sample with polymerase chain reaction reagents specific for said target nucleic acid, said polymerase chain reaction reagents including at least two polymerase chain reaction primers, a polymerase enzyme, and an internal fluorescent oligonucleotide probe, said oligonucleotide probe further comprising:
(i) a sequence capable of hybridizing to a portion of said target nucleic acid;
(ii) a fluorescer molecule attached to the 3′ end of the oligonucleotide;
(iii) a quencher molecule attached to the 5′ end of said oligonucleotide capable of substantially quenching said fluorescer molecule when said oligonucleotide is intact, wherein said fluorescer molecule becomes substantially unquenched when said oligonucleotide probe is cleaved by DNA polymerase during amplification of the target nucleic acid; and
(iv) said 3′ end is impervious to the 5′→3′ extension activity of said polymerase enzyme; and
(b) amplifying the target nucleic acid by thermal cycling, wherein said thermal cycling is sufficient to amplify said target nucleic acid; and
(c) measuring the level of fluorescence in said sample subsequent to thermal cycling, and further wherein the level of fluorescence is correlated to the amount of target nucleic acid present in the sample, thereby quantitatively detecting a target nucleic acid encoding a mammalian Lhx3 in a sample.

25. The method of claim 24, wherein said polymerase chain reaction primer is selected from the group consisting of a nucleic acid having the sequence SEQ ID NO:44, and a nucleic acid having the sequence of SEQ ID NO:45.

26. A kit comprising a compound which specifically hybridizes with a nucleic acid having at least about 88.5% homology to a nucleic acid having the sequence of at least one of SEQ ID NO:7, SEQ ID NO:9, and SEQ ID NO:11, and an instructional material for the use thereof.

27. The kit of claim 26, wherein said compound is selected from the group consisting of a nucleic acid and an antibody.

28. The kit of claim 27, wherein said nucleic acid is selected from the group consisting of a polymerase chain reaction primer, and an internal oligonucleotide probe.

29. A kit for detecting a nucleic acid encoding a mammalian Lhx3, said kit comprising a first nucleic acid having the sequence selected from the group consisting of SEQ ID NO:34, and SEQ ID NO:36, and a second nucleic acid having the sequence selected from the group consisting of SEQ ID NO:35, and SEQ ID NO:37, and an instructional material for the use thereof.

30. The kit of claim 29, further comprising an internal oligonucleotide probe complementary to at least a portion of a nucleic acid having the sequence of from about nucleotide 1 to about nucleotide 8867 of SEQ ID NO:22.

31. The kit of claim 30, wherein said oligonucleotide probe has the sequence selected from the group consisting of SEQ ID NO:38, SEQ ID NO:39, and SEQ ID NO:40.

32. A method of quantitatively detecting a nucleic acid encoding a mammalian Lhx3 a in a sample, said method comprising
(a) contacting a target nucleic acid encoding a mammalian Lhx3a present in a sample with polymerase chain reaction reagents specific for said target nucleic acid, said polymerase chain reaction reagents including at least two polymerase chain reaction primers, a polymerase enzyme, and an internal fluorescent oligonucleotide probe, the oligonucleotide probe further comprising:
(i) a sequence capable of hybridizing to a portion of said target nucleic acid wherein said portion is unique to Lhx3a;
(ii) a fluorescer molecule attached to a 5′ end of said oligonucleotide;
(iii) a quencher molecule attached to a 3′ end of said oligonucleotide capable of substantially quenching said fluorescer molecule when said oligonucleotide is intact, wherein said fluorescer molecule becomes substantially unquenched when said oligonucleotide probe is cleaved by DNA polymerase during amplification of the target nucleic acid; and
(iv) a 3′ end which is impervious to the 5′→3′ extension activity of the DNA polymerase; and
(b) amplifying the target nucleic acid by thermal cycling, wherein said thermal cycling is sufficient to amplify said target nucleic acid; and
(c) measuring the level of fluorescence in said sample subsequent to thermal cycling, and further wherein the level of fluorescence is correlated to the amount of target nucleic acid present in the sample, thereby quantitatively detecting a target nucleic acid encoding a mammalian Lhx3a in a sample.

33. The method of claim 32, wherein said polymerase chain reaction primer is selected from the group consisting of a first nucleic acid having a sequence selected from the group consisting of SEQ ID NO:34, and a second nucleic acid having a sequence selected from the group consisting of SEQ ID NO:35.

34. A kit for quantitatively detecting a nucleic acid encoding a mammalian Lhx3a, said kit comprising a polymerase chain reaction primer having the sequence selected from the group consisting of SEQ ID NO:34, and SEQ ID NO:35, and an instructional material for the use thereof.

35. The kit of claim 34, further comprising an internal oligonucleotide probe which specifically hybridizes with a nucleic acid molecule comprising a nucleic acid having the sequence of SEQ ID NO:38.

36. The kit of claim 35, wherein said internal oligonucleotide probe has the sequence SEQ ID NO:40.

37. A kit for quantitatively detecting a nucleic acid encoding a mammalian Lhx3b, said kit comprising a polymerase chain reaction primer having the sequence selected from the group consisting of SEQ ID NO:36, and SEQ ID NO:37, and an instructional material for the use thereof.

38. The kit of claim 37, further comprising an internal oligonucleotide probe which specifically hybridizes with a nucleic acid molecule having the sequence of SEQ ID NO:39.

39. A method of quantifying the level of Lhx3 expressed in a cell, the method comprising, reverse transcribing any ribonucleic acid encoding Lhx3 present in a cell, amplifying any deoxyribonucleic acid encoding Lhx3 produced by reverse transcription, and detecting any amplified deoxyribonucleic acid formed using quantitative sequence detection, thereby quantifying the level of Lhx3 expressed in a cell.

40. The method of claim 39, wherein said Lhx3 is selected from the group consisting of porcine Lhx3a, porcine Lhx3b, human Lhx3a, and human Lhx3b.

41. The method of claim 39, wherein said amplification comprises amplification using polymerase chain reaction wherein said polymerase chain reaction comprises a primer having a sequence selected from the group consisting of SEQ ID NO:41, and SEQ ID NO:35.

42. The method of claim 39, wherein said detection comprises contacting any deoxyribonucleic acid produced with an internal fluorescent oligonucleotide probe which specifically hybridizes with a nucleic acid of claim 1.

43. A kit for detecting a nucleic acid encoding a mammalian Lhx3a, said kit comprising a first nucleic acid having the sequence SEQ ID NO:34, and a second nucleic acid having the sequence SEQ ID NO:35, and an instructional material for the use thereof.

44. The kit of claim 43, further comprising an internal oligonucleotide probe complementary to at least a portion of a nucleic acid having the sequence of SEQ ID NO:9.

45. A kit for detecting a nucleic acid encoding a mammalian Lhx3b, said kit comprising a first nucleic acid having the sequence SEQ ID NO:36, and a second nucleic acid having the sequence SEQ ID NO:37, and an instructional material for the use thereof.

46. The kit of claim 45, further comprising an internal oligonucleotide probe complementary to at least a portion of a nucleic acid having the sequence of SEQ ID NO:11.

47. The kit of claim 46, wherein the oligonucleotide probe has the sequence SEQ ID NO:40.

48. A method of identifying a compound that affects expression of human Lhx3 in a cell, said method comprising contacting a cell with a compound and comparing the level of expression of Lhx3 in said cell contacted with said compound with the level of expression of Lhx3 is an otherwise identical cell, wherein a higher or lower level of expression of Lhx3 is said cell contacted with said compound compared with the level of expression of Lhx3 in said otherwise identical cell not contacted with said compound, is an indication that said compound affects expression of Lhx3 in a cell.

49. The method of claim 48, wherein said human Lhx3 is selected from the group consisting of hLhx3a, and hLhx3b.

50. A method of identifying a compound that affects the level of expression of Lhx3a but not the level of expression of Lhx3b in a cell, said method comprising contacting a cell with a compound and comparing the level of expression of Lhx3a in a cell with the level of expression of Lhx3a in an otherwise identical cell not contacted with said compound, and further comparing the level of expression of Lhx3b in said cell contacted with said compound with the level of expression of Lhx3b in said otherwise identical not contacted with said compound, wherein a higher or lower level of expression of Lhx3a in said cell contacted with said compound compared with the level of expression of Lhx3a in said otherwise identical cell not contacted with said compound, and further wherein there is no detectable change in the level of expression of Lhx3b in said cell contacted with said compound compared to the level of expression of Lhx3b in said otherwise identical cell not contacted with said compound, is an indication that said compound affects the level of expression of Lhx3a but not the level of expression of Lhx3b in a cell.

51. A method of identifying a compound that affects the level of expression of Lhx3b but not the level of expression of Lhx3a in a cell, said method comprising contacting a cell with a compound and comparing the level of expression of Lhx3b in a cell with the level of expression of Lhx3b in an otherwise identical cell not contacted with said compound, and further comparing the level of expression of Lhx3a in said cell contacted with said compound with the level of expression of Lhx3a in said otherwise identical not contacted with said compound, wherein a higher or lower level of expression of Lhx3b in said cell contacted with said compound compared with the level of expression of Lhx3b in said otherwise identical cell not contacted with said compound, and further wherein there is no detectable change in the level of expression of Lhx3a in said cell contacted with said compound compared to the level of expression of Lhx3a in said otherwise identical cell not contacted with said compound, is an indication that said compound affects the level of expression of Lhx3b but not the level of expression of Lhx3a in a cell.

52. A method of identifying a compound which affects the activity of human Lhx3 in a cell, said method comprising contacting a cell with a compound and comparing the level of activity of Lhx3 in said cell contacted with said compound with the level of activity of Lhx3 in an otherwise identical cell not contacted with said compound, wherein a higher or lower level of activity of Lhx3 is said cell contacted with said compound compared with the level of activity of Lhx3 in said otherwise identical cell not contacted with said compound, is an indication that said compound affects the activity of Lhx3 in a cell.

53. The method of claim 52, wherein said activity of said human Lhx3 is selected from the group consisting of the ability of hLhx3 to induce expression of a reporter gene operably linked to a αGSU promoter, the ability of hLhx3 to induce expression of a reporter gene operably linked to a TSHβ promoter, the ability of hLhx3 to bind to the −350 to −323 bp region of murine αGSU promoter, the ability to activate the gene encoding Pit-1, and the ability to activate the gene encoding prolactin.

54. The method of claim 52, wherein said human Lhx3 is selected from the group consisting of Lhx3a, and Lhx3b.

55. A method of identifying a compound which affects the level of activity of Lhx3 in a cell, said method comprising
(a) transfecting a cell with a vector encoding Lhx3;
(b) co-transfecting said cell transfected with a reporter gene construct wherein said construct comprises a reporter gene operably linked to a promoter/regulatory sequence that is trans-activated by Lhx3;
(c) contacting said cell of (b) with a test compound;
(d) measuring the level of activity of Lhx3 in said cell of (b) before and after contacting said cell with said compound;
(f) comparing the level of activity of Lhx3 in said cell contacted with said compound with the level of activity of Lhx3 in said cell prior to or in the absence of contacting said cell with said compound;
wherein a higher or lower level of activity of Lhx3 in said cell contacted with said compound compared with the level of activity of Lhx3 in said cell prior to or in the absence of contacting said cell with said compound is an indication that said compound affects the level of activity of Lhx3 in a cell.

56. A method of identifying a compound which affects the level of activity of Lhx3a but not the level of activity of Lhx3b in a cell, said method comprising
(a) transfecting a cell having no detectable endogenous Lhx3a activity with an expression vector encoding Lhx3a;
(b) transfecting an otherwise identical cell said cell having no detectable endogenous Lhx3b activity with an expression vector encoding Lhx3b;
(c) co-transfecting each of said cells in (a) and (b) with a reporter gene construct comprising a reporter gene operably linked to a promoter/regulatory sequence trans-activated by Lhx3a and Lhx3b;
(d) contacting each of said cells of (c) with a test compound;
(e) measuring the level of Lhx3a activity in each of said cells of (c) before and after contacting each of said cells with said test compound;
(f) measuring the level of Lhx3b activity in each of said cells of (c) before and after contacting each of said cells with said test compound;
(g) comparing the level of activity of Lhx3a in each of said cells of (c) contacted with said compound to the level of activity of Lhx3a in each of said cells prior to or in the absence of contacting each of said cells with said test compound;
(h) comparing the level of activity of Lhx3b in each of said cells of (c) contacted with said test compound to the level of activity of Lhx3b in each of said cells prior to or in the absence of contacting each of said cells with said test compound,
wherein a higher or lower level of activity Lhx3a in each of said cells of (c) contacted with said test compound compared to the level of activity of Lhx3a in each of said cells prior to or in the absence of contacting each of said cells with said test compound and further wherein there is no detectable change in the level of Lhx3b activity in each of said cells of (c) contacted with said test compound compared to the level of Lhx3b activity in each of said cells of (c) prior to or in the absence of contacting each of said cells with said test compound, is an indication that said test compound affects the level of Lhx3a activity but not the level of Lhx3b activity in a cell.

57. A method of identifying a compound which affects binding of Lhx3 to a nucleic acid that specifically binds with Lhx3, said method comprising administering a compound into an extract wherein said extract comprises Lhx3 and a nucleic acid that specifically binds with Lhx3, and comparing the level of binding of Lhx3 with said nucleic acid in said extract comprising said compound with the level of binding of Lhx3 with said nucleic acid in an otherwise identical extract which does not comprise said compound, wherein a higher or lower level of binding of Lhx3 with said nucleic acid in said extract comprising said compound compared to the level of binding of Lhx3 to said nucleic acid in said otherwise identical extract not comprising said compound, is an indication that said compound affects binding of Lhx3 with a nucleic acid that specifically binds with Lhx3.

58. The method of claim 57, wherein said Lhx3 is selected from the group consisting of human Lhx3a, human Lhx3b, porcine Lhx3a and porcine Lhx3b.

59. The method of claim 58, wherein said nucleic acid that specifically binds with Lhx3 is selected from the group consisting of a nucleic acid having nucleotides −350 to −323 of murine αGSU promoter (SEQ ID NO:42), and a nucleic acid encoding the Lhx3 consensus binding sequence (SEQ ID NO:43).

60. A method of identifying a compound that affects Lhx3 induction of a pituitary trophic hormone gene promoter, said method comprising
(a) transfecting a cell with a reporter gene construct wherein said construct comprises a thyroid-stimulating hormone beta promoter sequence operably linked to a reporter gene;
(b) transfecting said cell of (a) with a hLhx3 expression vector;
(c) contacting said cell of (b) with a pituitary transcription factor which synergizes with Lhx3;
(d) contacting said cell of (c) with a compound;
(e) assessing the level of expression of said reporter gene where the level of expression of said reporter gene is correlated to the level of Lhx3 induction of said promoter; and
(f) comparing the level of expression of said reporter gene in said cell contacted with said compound with the level of expression of said reporter gene in an otherwise identical cell not contacted with said compound,
wherein in a higher or lower level of expression of said reporter gene in said cell contacted with said compound compared with the level of expression of said reporter gene in said otherwise identical cell not contacted with said compound, is an indication that said compound affects Lhx3 induction of a pituitary trophic hormone gene promoter in a cell.

61. The method of claim 60, wherein said pituitary trophic hormone gene promoter is selected from the group consisting of a thyroid-stimulating hormone beta promoter, an alpha-glycoprotein subunit promoter, and a prolactin promoter.

62. The method of claim 60, wherein said pituitary transcription factor which synergizes with Lhx3 is selected from the group consisting of Pit-1, Pitx1/P-Otx, and thyrotrope embryonic factor.

63. The method of claim 60, wherein said Lhx3 is selected from the group consisting of Lhx3a, Lhx3b, pLhx3a, and pLhx3b.

64. A method of identifying a human patient afflicted with a disease, disorder or condition associated with altered expression of Lhx3, said method comprising detecting the level of Lhx3 expression in a human and comparing said level of expression of Lhx3 in said human with the level of expression of Lhx3 in a normal human not afflicted with a disease, disorder or condition associated with altered expression of Lhx3, thereby detecting a human patient afflicted with a disease, disorder or condition associated with altered expression of Lhx3.

65. A method of identifying a human patient afflicted with a disease, disorder or condition associated with altered level of binding of Lhx3 to a nucleic acid that specifically binds with Lhx3, said method comprising detecting the level of Lhx3 binding with a nucleic acid that specifically binds with Lhx3 in a human and comparing said level of Lhx3 binding with a nucleic acid that specifically binds with Lhx3 in said human with the level of Lhx3 binding with a nucleic acid that specifically binds with Lhx3 in a normal human not afflicted with a disease, disorder or condition associated with altered level of Lhx3 binding with a nucleic acid that specifically binds with Lhx3, wherein a higher or lower level of Lhx3 binding with a nucleic acid that specifically binds with Lhx3 in said human compared with the level of Lhx3 binding with a nucleic acid that specifically binds with Lhx3 in said human not afflicted with a disease, disorder or condition associated with altered level of Lhx3 binding with a nucleic acid that specifically binds with Lhx3, is an indication that said human is afflicted with a disease, disorder or condition associated with altered level of Lhx3 binding with a nucleic acid that specifically binds with Lhx3, thereby detecting a human patient afflicted with a disease, disorder or condition associated with altered level of Lhx3 binding with a nucleic acid that specifically binds with Lhx3.

66. The method of claim 65, wherein said nucleic acid that specifically binds with Lhx3 is selected from the group consisting of nucleotides −350 to −323 of murine αGSU promoter, and the Lhx3 consensus binding sequence.

67. A method of detecting a mutation in a Lhx3 allele in a human, said method comprising comparing the nucleic acid sequence encoding Lhx3 of a human suspected of having a mutation in Lhx3 with the nucleic acid sequence encoding Lhx3 obtained from a normal human not having a mutation in Lhx3, wherein any difference between said nucleic acid sequence of said human suspected of having a mutation in Lhx3 and said nucleic acid sequence encoding Lhx3 of said normal human not having a mutation in Lhx3 detects a mutation in a Lhx3 allele in said human.

68. A method of detecting a mutation in a Lhx3 allele in a human, said method comprising comparing the genomic nucleic acid sequence encoding Lhx3 of a human suspected of having a mutation in Lhx3 with the genomic nucleic acid sequence encoding Lhx3 obtained from a normal human not having a mutation in Lhx3, wherein any difference between said genomic nucleic acid sequence of said human suspected of having a mutation in Lhx3 and said genomic nucleic acid sequence encoding Lhx3 of said normal human not having a mutation in Lhx3 detects a mutation in a Lhx3 allele in said human.
